(19) United States
(12) Patent Application Publication
(10) Pub. No.: US 2022/0378611 A1
(43) Pub. Date: Dec. 1, 2022

(54) DRUG THERAPY DELIVERY SYSTEMS AND METHODS

(71) Applicant: W. L. Gore & Associates, Inc., Newark, DE (US)

(72) Inventors: Jérôme S. Conia, Phoenix, AZ (US); Peter J. Roeber, Oxford, PA (US); Jeffrey C. Towler, Wilmington, DE (US)

(21) Appl. No.: 17/825,909

(22) Filed: May 26, 2022

Related U.S. Application Data
(60) Provisional application No. 63/194,152, filed on May 27, 2021.
(60) Provisional application No. 63/345,595, filed on May 25, 2022.

(51) Int. Cl. A61F 9/00; A61K 9/00

(52) U.S. Cl. CPC A61F 9/0017; A61K 9/0051

(57) ABSTRACT

An implantable delivery device for dispensing a medicament that includes a first microporous material that is bonded to a second microporous material. The first microporous material has a first microporous layer including a plurality of pores sized to permit tissue ingrowth and a second microporous layer including a plurality of pores sized to permit tissue ingrowth. The second microporous material has a third microporous layer including a plurality of pores sized to resist tissue ingrowth and a fourth microporous layer including a plurality of pores sized to permit tissue ingrowth. The second microporous layer is bonded to the third microporous layer to thereby form a reservoir for receiving the medicament. The first and second microporous materials are configured to meter a rate at which the medicament is dispensed from the reservoir when the delivery device is implanted.

### CROSS-REFERENCE TO RELATED APPLICATION

This application claims the benefit of Provisional Application No. 63/194,152, filed May 27, 2021, and also claims the benefit of Provisional Application No. 63/345,595, filed May 25, 2022, which are incorporated herein by reference in their entireties for all purposes.

### BACKGROUND

Aqueous humor is a fluid that fills the anterior chamber of the eye and contributes to the intraocular pressure or fluid pressure inside the eye. Ocular hypertension is a condition in the eye where the intraocular pressure or fluid pressure inside the eye is elevated. Untreated ocular hypertension can lead to disease, including glaucoma, which can result in a gradual and sometimes permanent loss of vision in the afflicted eye.

Many attempts have been made to treat ocular hypertension, and glaucoma in particular. Such attempts include surgical procedures that involve implantation of drainage devices designed to lower the intraocular pressure of the afflicted eye, as well as medicament administration. The goal of these treatments is to improve quality of life and to preserve visual function through a reduction of the intraocular pressure.

Though medicament administration is typically in the form of eyedrops that must be self-administered by the patient, implantable extended drug delivery devices can be employed in certain instances. Implantable extended drug delivery devices typically reside either on the exterior of the eye (e.g., extraocular approaches), or are alternatively implanted within the anterior chamber of the eye (intracameral approaches).

Extraocular approaches to medicament delivery present a variety of challenges. To be effective, the extraocular approach requires transporting, via the biological processes of the eye, a sufficient amount of the medicament through the conjunctival layer of the eye and into the anterior chamber of the eye. Obvious natural mechanisms such as the continual flushing mechanism of the human tear film, as well as the natural barrier to the interior of the eye formed by the conjunctiva, complicate the effectiveness of extraocular approaches, resulting in sub-optimal dose delivery over time. Extraocular approaches therefore sometimes include administration of an excessive amount of the medicament to extend a period of efficacy.

Intracameral approaches, on the other hand, are more invasive approaches requiring a puncture through the various tissue layers of the eye to gain access to, and placement of, the device within the anterior chamber of the eye. Intracameral approaches are additionally complicated where the medicament is administered in association with a device that is absorbable (bioabsorbable), as the degrading nature of the device may lead to the device dislodging and floating within the anterior chamber. Moreover, device removal and repeated replacement requires trauma to the tissues of the eye.

Examples of the above-referenced approaches that have been established for treating ocular conditions are described herein.

For example, Allergan has developed a bioabsorbable pellet for drug delivery in the anterior chamber of the eye and sold under the trademark name DURYSTA. The delivery method is described in at least U.S. Pat. No. 10,398,707 to Allergan Inc, and filed Oct. 2, 2022. The medicament, bimatoprost, is loaded within the bioabsorbable pellet which is implanted into the eye and dissolves within the anterior chamber to deliver the loaded bimatoprost. However, this can negatively affect the endothelial cells in the cornea due to possible abrasion during the dissolving process.

Similarly, Allergan Inc. is developing a device described as the “ENV515,” which is a fully bioabsorbable device loaded with travoprost, as disclosed in U.S. Patent Application No. US 2022/0080049, filed Mar. 25, 2021. After implantation of the device into the anterior chamber, water will cause at least a portion of the device to bioerode through which the active agent is released. However, this may result in potential damage to the endothelial cells due to abrasion during bioerosion of the device.

Glaukos, Inc. has a device under development referred to as the “iDose,” which is a metal plug that is inserted into the anterior chamber for delivering a glaucoma treatment or medicament, such as travoprost. This device is described in at least U.S. Patent. App. No. 2021/0315806A1 to Glaukos Corp and filed on Mar. 26, 2021. However, this device is not bioabsorbable or refillable while placed within the eye. As such, it requires removing the device, refilling the device, and reimplanting the device. This results in increased hassle for the patient and requires subsequent procedures.

Allergan has additionally developed an extraocular ring that elutes a glaucoma drug onto the surface of the eye. The drug eluded by the ring may be bimatoprost. The device is a relatively large silicone ring positioned within the ocular fornix. The drug is released from the ring and transports through the cornea for drug delivery. However, this delivery method is not as effective as direct delivery may cause discomfort to the patient as the device sits around the eye rather than within the eye.

Still with reference to established glaucoma treatments, Mati Therapeutics has developed a punctal plug that sits on the outside of a users' eye in the punctum. The drug may then leach out of the device and through the eye to reach the delivery target. The device is described in at least U.S. Pat. No. 9,216,108 to Mati Therapeutics, and filed on Feb. 17, 2009. Similar to the above-described extraocular device, this may provide discomfort to the user as the device sits within and around the eye. This method is also not as effective for drug delivery as direct delivery drug delivery can be.

Medicament delivery methods have been established for various other ocular diseases, as well. For example, Genentech has established a drug delivery device for wet macular degeneration through a port delivery system, which is described in U.S. Pat. No. 10,398,593 to ForSight Vision4 Inc. and filed on Dec. 21, 2016. The port delivery system requires invasion into the eye, as the access port sits underneath the conjunctiva for refilling of the drug. This may cause patient discomfort and potentially any other complications associated with invasive drug delivery.

Treating or preventing eye diseases may be accomplished by means of genetic therapy, whereby a disorder is corrected or influenced by altering the genetic disposition of target cells or target tissues in the eye. Genome therapy may be accomplished in a number of ways. Gene transfer allows for the addition of specific genetic material needed to compensate for a missing protein function. Genome editing enables to precisely correct the DNA alteration responsible for a disorder. By introducing a new genetic capability, the diseased tissue may then produce needed protein.

Gene therapy is achieved by transduction using genetically engineered vectors. These vectors are preferably modified viruses or virus capsids that are used to transport distinct genetic material to the targeted cells and tissues. Viruses are helpful since they can deliver the nucleic acid material by infecting the cells. Gene therapy strategies may anticipate integration of the new genetic material within DNA of the host cells, such as enabled by modified retroviruses, or introduction of the new genetic material within the nucleus of the host cell but without chromosomal integration, as it is supported by adenoviruses. Using unmodified viruses is not without drawbacks since the immune system of a patient could respond to the presence of the viral material. Furthermore, clinical complications could result from the systemic dissemination of unmodified viral particles.

As adenoviruses do, adeno-associated virus (AAV) vectors are capable of transducing various host cell types without integrating the host genome. Adeno-associated viruses are not known to lead to serious complication or illness. The immune response the viruses may elicit is inconsequential. AAV vectors have gained the reputation of being the optimum transduction vector. The AAV vectors are small and lack pathogenicity while retaining the capacity to infect non-dividing cells. At least these attributes make AAV vectors very attractive for use in gene therapy. AAV capsids have proven successful for gene therapy in a number of preclinical and clinical applications, gaining regulatory approvals globally. In addition, controlled gene expression and regulation has been proposed using an additional inducer, such as oral inducer (developed by MeiraGTx). Advances in gene therapy using AAV vectors have shown promise, particularly for the treatment of retinal disease, and are being explored by a number of companies, for example Adverum and RegenXBio. The goal of gene therapy is for the transduced retinal cells to express the therapeutic DNA sequence over a long period of time.

In the ophthalmic arena, retinal gene therapy is believed to be applicable to several conditions, such as preventing glaucomatous neurodegeneration and protecting retinal ganglion cells (non-IOP-related interventions), or to support the photoreceptors and retinal pigment epithelium for the treatment of age-related macular degeneration (AMD) and retinitis pigmentosa (RP). Other targeted diseases include geographic atrophy (GA), diabetic macular edema (DME), and diabetic retinopathy (DR). Once the cells are transduced, the AAV is expected to persist for an extended time and to provide expression of the therapeutic DNA sequence for several months and potentially years.

The gene therapy vectors are commonly administered by means of bolus injection, either focal sub-retinal, intravitreal, or suprachoroidal. These administration routes present a degree of invasiveness that can lead to clinical complications. Furthermore, they are inadequately applicable to the progressive introduction of the transduction vectors over an extended period and fail to administer the therapy gradually.

In contrast, the drug therapy delivery systems and methods disclosed herein introduce unique benefits for the administration of gene therapy and enabling the durable and sustained distribution of the transducing vectors over time and over larger peripheral areas. Hence, the systems and methods disclosed herein enhance cellular uptake and dosage control while optimizing tissue targeting. System embodiment enables sub-conjunctival or sub-Tenon administration routes designed to prevent vascular clearance though the conjunctiva, and the minimally invasive implant procedure avoids injury to and clearance by the choriocapillaris. Exposure to preexisting neutralizing antibodies (Nabs) to AAV capsid antigens is thus minimized.

In addition, the drug therapy delivery systems and methods disclosed herein are consistent with alternative methods for gene therapy such as those anticipating for the delivery of genetic material to host cells without viruses. Polymer-based nanoparticles (<100 nm), liposomes, and compacted DNA or RNA nanoparticles (DNPs) can be tailored to various release rates, hydrophilicity and lipophilicity, and designed with enhanced specificity for a particular cell type. Nanoparticle gene delivery is less likely to elicit a reaction from the immune system. Also, these vectors can accommodate a larger carrying capacity (i.e.: 20 kb) and larger genes as compared to AAVs, which can contain plasmid sizes of up to 5 kb.

Through the above publications provide some examples of drugs and/or delivery systems that may be applied to ocular conditions, various other approaches have been pursued. There remains a need for a minimally invasive drug-delivery method that provides localized ocular drug delivery at a measured delivery rate over a desired period of time without patient participation and without breaching the blood ocular barrier.

### SUMMARY

According to an example (“Example 1”) a method of treating diseases of the eye, the method comprising: selecting an implantable delivery device that includes a first microporous material that is bonded to a second microporous material, the first microporous material having a first microporous layer including a plurality of pores sized to permit tissue ingrowth and a second microporous layer including a plurality of pores sized to permit tissue ingrowth, the second microporous material having a third microporous layer including a plurality of pores sized to resist tissue ingrowth and a fourth microporous layer including a plurality of pores sized to permit tissue ingrowth, the second microporous layer being bonded to the third microporous layer to thereby form a reservoir for receiving at least one medicament, wherein the first and second microporous materials are configured to meter a rate at which the medicament is dispensed from the reservoir when the delivery device is implanted; filling the reservoir with a medicament for treating one or more diseases of the eye; implanting the implantable delivery device at an implantation location; and allowing the medicament to dispense from the reservoir to one or more treatment sites.

According to another example (“Example 2”), further to Example 1, wherein the implantation location is an anterior reservoir location.

According to another example (“Example 3”), further to Example 2, wherein the one or more eye diseases includes at least one of glaucoma, keratitis, dry eye, or presbyopia.

According to another example (“Example 4”), further to Example 3, wherein when the one or more diseases is glaucoma, the medicament has an API class that includes at least one of prostaglandins, prostaglandin structural analogs, beta blockers, alpha agonist, or carbonic anhydrase inhibitors.

According to another example (“Example 5”), further to Example 3, wherein when the one or more diseases is keratitis, the medicament has an API class that includes at least one of antibiotics, steroids, or antifungal agents.

According to another example (“Example 6”), further to Example 3, wherein when the one or more diseases is dry eye, the medicament has an API class that corresponds to at least one of prostaglandins, beta blockers, alpha agonist, or carbonic anhydrase inhibitors.

According to another example (“Example 7”), further to Example 3, wherein when the one or more diseases is presbyopia, the medicament has an API class that includes miotics.

According to another example (“Example 8”), further to Example 1, wherein the implantation location is a posterior reservoir location.

According to another example (“Example 9”), further to Example 1, wherein the one or more eye diseases includes at least one of macular degeneration, geographic atrophy lesion, macular edema, uveitis, retinitis, keratitis, retinoblastoma, central retinal vein occlusion, or branch retinal vein occlusion.

According to another example (“Example 10”), further to Example 9, wherein when the one or more diseases is macular degeneration, the medicament has an API class that includes monoclonal antibodies, antibody mimetic proteins, peptides, or small binding molecules.

According to another example (“Example 11”), further to Example 9, wherein when the one or more diseases is macular edema, the medicament has an API class that includes monoclonal antibodies, antibody mimetic proteins, peptides, small binding molecules, or steroids.

According to another example (“Example 12”), further to Example 9, wherein when the one or more diseases is uveitis, the medicament has an API class that includes corticosteroid.

According to another example (“Example 13”), further to Example 9, wherein when the one or more diseases is retinitis, the medicament has an API class that includes antibiotics or antivirals.

According to another example (“Example 14”), further to Example 9, wherein when the one or more diseases is retinoblastoma, the medicament has an API class that includes cytotoxic chemotherapy compounds.

According to another example (“Example 15”), further to Example 9, wherein when the one or more diseases is central retinal vein occlusion or branch retinal vein occlusion, the medicament has an API class that includes monoclonal antibodies or steroids.

According to another example (“Example 16”), further to Example 1, wherein the implantation location is an anterior-posterior reservoir location.

According to another example (“Example 17”), further to Example 16, wherein the one or more eye diseases includes at least one of macular degeneration, geographic atrophy lesion, macular edema, uveitis, retinitis, central retinal vein occlusion, or branch retinal vein occlusion.

According to another example (“Example 18”), further to Example 17, wherein when the one or more diseases is macular degeneration, the medicament has an API class that includes monoclonal antibodies.

According to another example (“Example 19”), further to Example 17, wherein when the one or more diseases is macular edema, the medicament has an API class that includes monoclonal antibodies or steroids.

According to another example (“Example 20”), further to Example 17, wherein when the one or more diseases is uveitis, the medicament has an API class that includes corticosteroid.

According to another example (“Example 21”), further to Example 17, wherein when the one or more diseases is retinitis, the medicament has an API class that includes antibiotics or antivirals.

According to another example (“Example 22”), further to Example 17, wherein when the one or more diseases is central retinal vein occlusion or branch retinal vein occlusion, the medicament has an API class that includes monoclonal antibodies or steroids.

According to another example (“Example 23”), further to Example 1, wherein the implantable delivery device includes a refillable reservoir and a delivery arm.

According to another example (“Example 24”), further to Example 23, wherein the method further includes refilling the refillable reservoir.

According to another example (“Example 25”), further to Example 1, wherein the implantable delivery device has one or more fill ports with which to refill the reservoir.

According to another example (“Example 26”), further to Example 1, wherein the implantable delivery device is configured to dispense medicament in a single direction.

According to another example (“Example 27”), further to Example 1, wherein the implantable delivery device is configured to dispense medicament in multiples directions.

According to another example (“Example 28”), further to Example 1, wherein the implantable delivery device has a plurality of chambers defined within the reservoir, and wherein first and second chambers in the plurality of chambers are in fluid communication with each other.

According to another example (“Example 29”), further to Example 1, wherein the implantable delivery device has a plurality of chambers defined within the reservoir, and wherein first and second chambers in the plurality of chambers are fluidly isolated from each other.

According to another example (“Example 30”), further to Example 1, wherein the implantable delivery device has a plurality of chambers defined within the reservoir, and wherein the implantable delivery device has one or more fill ports with which to refill the reservoir.

According to another example (“Example 31”), further to Example 30, wherein the number of fill ports corresponds to the number of chambers.

According to another example (“Example 32”), further to Example 31, wherein implanting the implantable delivery device at an implantation location includes arranging the device at the implantation such that the implantable delivery device is subconjunctival.

According to another example (“Example 33”), further to Example 1, wherein implanting the implantable delivery device at an implantation location includes arranging the device at the implantation such that the implantable delivery device is suprachoroidal.

According to another example (“Example 34”), further to Example 1, wherein the implantation location is an anterior-intravitreal reservoir location.

According to another example (“Example 35”), further to Example 34, wherein the one or more eye diseases includes at least one of macular degeneration, geographic atrophy lesion, macular edema, uveitis, retinitis, central retinal vein occlusion, or branch retinal vein occlusion.

According to another example (“Example 36”), further to Example 35, wherein when the one or more diseases is macular degeneration, the medicament has an API class that includes monoclonal antibodies.

According to another example (“Example 37”), further to Example 35, wherein when the one or more diseases is macular edema, the medicament has an API class that includes monoclonal antibodies or steroids.

According to another example (“Example 38”), further to Example 35, wherein when the one or more diseases is uveitis, the medicament has an API class that includes corticosteroid.

According to another example (“Example 39”), further to Example 35, wherein when the one or more diseases is retinitis, the medicament has an API class that includes antibiotics or antivirals.

According to another example (“Example 40”), further to Example 35, wherein when the one or more diseases is central retinal vein occlusion or branch retinal vein occlusion, the medicament has an API class that includes monoclonal antibodies or steroids.

According to another example (“Example 41”), further to Example 23, wherein the implantation location is an anterior-anterior-chamber reservoir location.

According to another example (“Example 42”), further to Example 41, wherein the one or more eye diseases includes at least one of macular degeneration, macular edema, uveitis, retinitis, central retinal vein occlusion, or branch retinal vein occlusion.

According to another example (“Example 43”), further to Example 42, wherein when the one or more diseases is glaucoma, the medicament has an API class that includes at least one of prostaglandins, beta blockers, alpha agonist, or carbonic anhydrase inhibitors.

According to another example (“Example 44”), further to Example 42, wherein when the one or more diseases is keratitis, the medicament has an API class that includes at least one of antibiotics, steroids, or antifungal agents.

According to another example (“Example 45”), further to Example 42, wherein when the one or more diseases is dry eye, the medicament has an API class that corresponds to at least one of prostaglandins, beta blockers, alpha agonist, or carbonic anhydrase inhibitors.

According to another example (“Example 46”), further to Example 42, wherein when the one or more diseases is presbyopia, the medicament has an API class that includes miotics.

According to another example (“Example 47”), further to Example 1, wherein gene therapy is deployed to prevent glaucomatous neurodegeneration, for the treatment of age-related macular degeneration, retinitis pigmentosa, geographic atrophy, diabetic macular edema, and diabetic retinopathy.

According to another example (“Example 48”), further to Example 1, wherein gene therapy is achieved by the sustained administration of either viral transducing vectors, adeno-associated virus (AAV) vectors, polymer-based nanoparticles, liposomes, or compacted nucleic acid nanoparticles.

According to another example (“Example 49”), a pharmaceutical composition includes at least one therapeutic agent and at least one additional material, wherein the pharmaceutical composition has an ability to adopt a first state and a second state, and to transition between the first state and the second state when exposed to a fluid. The pharmaceutical composition further includes wherein in the first state of the pharmaceutical composition does not allow movement of the at least one therapeutic agent through the additional material and in the second state the pharmaceutical composition permits movement of the at least one therapeutic agent through the additional material.

According to another example (“Example 50”), further to Example 49, the at least one additional material includes a polymer, optionally comprising bioabsorbable microparticles.

According to another example (“Example 51”), further to Example 49, in the second state, the at least one therapeutic agent is released from the at least one additional material at a predetermined rate.

According to another example (“Example 52”), further to Example 49, the bioabsorbable microparticles have an average size of between 15 microns to 25 microns.

According to another example (“Example 53”), further to Example 49, in the second state, the composition is capable of fluid absorption to release the therapeutic agent.

According to another example (“Example 54”), further to Example 49, the therapeutic agent comprises at least one of prostaglandin analogs, beta-blockers, alpha-2-agonists, and carbonic anhydrase inhibitors.

According to another example (“Example 55”), further to Example 54, the therapeutic agent comprises at least one of latanoprost, timolol, brimonidine, or dorzolamide.

According to another example (“Example 56”), further to Example 49, the first state includes a non-therapeutic state and wherein the second state comprises a therapeutic state.

According to another example (“Example 57”), a pharmaceutical composition includes at least one therapeutic agent and at least one additional material, wherein the pharmaceutical composition has an ability to adopt a first state and a second state, and to transition between the first state and the second state when exposed to a fluid. The composition further includes wherein in the first state, the pharmaceutical composition includes a non-therapeutic state.

According to another example (“Example 58”), further to Example 57, the second state comprises a therapeutic state.

According to another example (“Example 59”), further to Example 57, the therapeutic agent comprises at least one of prostaglandin analogs, beta-blockers, alpha-2-agonists, and carbonic anhydrase inhibitors.

According to another example (“Example 60”), further to Example 57, the additional material comprises a polymer, optionally comprising bioabsorbable materials.

According to another example (“Example 61”), a pharmaceutical composition disposed in a reservoir defined by at least one additional material includes at least one therapeutic agent, wherein the pharmaceutical composition has an ability to adopt a first state and a second state, and to transition between the first state and the second state when exposed to a fluid. The composition further includes wherein in the first state of the pharmaceutical composition the at least one additional material inhibits movement of the at least one therapeutic agent through the additional material and in the second state of the pharmaceutical composition the at least one additional material permits movement of the at least one therapeutic agent through the additional material.

According to another example (“Example 62”), further to Example 61, the transition between the first state and the second state is initiated by the at least one additional material permitting the passage of the fluid from an external environment to the pharmaceutical composition in the first state.

According to another example (“Example 63”), further to Example 61, the at least one additional material comprises a polymer, optionally comprising bioabsorbable microparticles.

According to another example (“Example 64”), further to Example 61, in the second state, the at least one therapeutic agent is released from the at least one additional material at a predetermined rate.

According to another example (“Example 65”), further to Example 61, the bioabsorbable microparticles have an average size of between 15 microns to 25 microns.

According to another example (“Example 66”), further to Example 61, in the second state, the composition is capable of fluid absorption to release the therapeutic agent.

According to another example (“Example 67”), further to Example 61, the therapeutic agent comprises at least one of prostaglandin analogs, beta-blockers, alpha-2-agonists, and carbonic anhydrase inhibitors.

According to another example (“Example 68”), further to Example 67, the therapeutic agent comprises at least one of latanoprost, timolol, brimonidine, or dorzolamide.

According to another example (“Example 69”), further to Example 61, the first state comprises a non-therapeutic state and wherein the second state comprises a therapeutic state.

According to another example (“Example 70”), a pharmaceutical composition disposed in a reservoir defined by at least one additional material includes at least one therapeutic agent wherein the pharmaceutical composition has an ability to adopt a first state and a second state, and to transition between the first state and the second state when exposed to a fluid. The composition further includes wherein in the first state of the pharmaceutical composition the pharmaceutical composition comprises a non-therapeutic state due to the at least one material inhibiting a movement of the at least one therapeutic agent in the first state.

According to another example (“Example 71”), further to Example 70, in the second state, the pharmaceutical composition comprises a therapeutic state due to the at least one material permitting the movement of the at least one therapeutic agent in the second state.

According to another example (“Example 72”), further to Example 70, the transition between the first state and the second state is initiated by the at least one additional material permitting the passage of the fluid from an external environment to the pharmaceutical composition in the first state.

According to another example (“Example 73”), further to Example 70, in the second state, the at least one therapeutic agent is released from the at least one additional material at a predetermined rate.

According to another example (“Example 74”), further to Example 70, the bioabsorbable microparticles have an average size of between 15 microns to 25 microns.

According to another example (“Example 75”), further to Example 70, in the second state, the composition is capable of fluid absorption to release the therapeutic agent.

According to another example (“Example 76”), further to Example 70, the therapeutic agent comprises at least one of prostaglandin analogs, beta-blockers, alpha-2-agonists, and carbonic anhydrase inhibitors.

According to another example (“Example 77”), further to Example 76, the therapeutic agent comprises at least one of latanoprost, timolol, brimonidine, or dorzolamide.

According to another example (“Example 78”), further to Example 78, the first state includes a non-therapeutic state and wherein the second state includes a therapeutic state.

According to another example (“Example 79”), an implantable medical device pre-loaded with a therapeutic agent includes a first microporous material bonded to a second microporous layer to define a reservoir disposed therebetween, the reservoir configured for containing the therapeutic agent, and the implantable medical device configured to release the therapeutic agent at a predetermined rate. The device further includes wherein the therapeutic agent is at least one of latanoprost, timolol, brimonidine, or dorzolamide.

According to another example (“Example 80”), an implantable medical device for delivering a therapeutic agent for treatment of a disease includes a first microporous material bonded to a second microporous layer to define a reservoir disposed therebetween, and the reservoir configured for containing a pharmaceutical composition and to deliver the therapeutic agent to a target site for treating the disease. The device further includes wherein the treatment is configured for treating one or more of glaucoma, macular degeneration, macular edema, retinitis, retinoblastoma, retinal vein occlusions, keratitis, and dry eye.

### BRIEF DESCRIPTION OF THE DRAWINGS

The accompanying drawings are included to provide a further understanding of the embodiments of the disclosure and are incorporated in and constitute a part of this specification, illustrate examples, and together with the description serve to explain the principles of the disclosure.

FIG. 1A is an illustration of a medicament delivery system implanted within an eye according to some embodiments.

FIG. 1B is a detailed view of FIG. 1A illustrating the medicament delivery system implanted within the eye according to some embodiments.

FIG. 2 is a cross-sectional view of the medicament delivery system illustrated in FIG. 4 taken along line 2-2 and in an inflated state according to some embodiments.

FIG. 3 is a cross-sectional view of the medicament delivery system illustrated in FIG. 4 taken along line 2-2 and in a deflated state according to some embodiments.

FIG. 4 is a front view of a medicament delivery system according to some embodiments.

FIG. 5 is a cross-sectional view of a medicament delivery system according to some embodiments.

FIG. 6 is a cross-sectional view of a medicament delivery system according to some embodiments.

FIG. 7 is a cross-sectional view of the medicament delivery system illustrated in FIG. 6 taken along line 7-7 according to some embodiments.

FIG. 8 is a cross-sectional view of a medicament delivery system according to some embodiments.

FIG. 9 is a cross-sectional view of the medicament delivery system illustrated in FIG. 8 taken along line 9-9 according to some embodiments.

FIG. 10 is an isometric view of an implantable delivery device with a reservoir and a delivery arm according to some embodiments.

FIGS. 10A and 10C are cross-sectional views of a medicament delivery system with an inflated reservoir according to some embodiments.

FIG. 10A-1 and FIG. 10A-2 are enlarged views of portions of the cross-sectional view of FIG. 10A.

FIG. 10C-1 and FIG. 10C-2 are enlarged views of portions of the cross-sectional view of FIG. 10C.

FIGS. 10B and 10D are detailed views of the reservoir in FIG. 1A being refilled according to some embodiments.

FIG. 11A is a plan view of an implantable delivery device reservoir in a first configuration with a main portion according to some embodiments.

FIG. 11B is a plan view of an implantable delivery device reservoir in a second configuration with main portion and a delivery arm according to some embodiments.

FIG. 11C is a plan view of an implantable delivery device reservoir in a third configuration with a main portion that includes a plurality of chambers and fill ports according to some embodiments.

FIG. 11D is a plan view of an implantable delivery device reservoir in a fourth configuration according to some embodiments that includes a plurality of chambers and fill ports according to some embodiments.

FIG. 12A is a plan schematic representation of an implantable delivery device with a single direction dispensing according to some embodiments.

FIG. 12B is a top view of the implantable delivery device in FIG. 12A according to some embodiments.

FIG. 12C is a plan schematic representation of an implantable delivery device with a single focused dispensing direction according to some embodiments.

FIG. 12D is a top view of the implantable delivery device in FIG. 12D according to some embodiments.

FIG. 12E is a plan schematic representation of an implantable delivery device with a multidirectional focused dispensing operation according to some embodiments.

FIG. 12F is a top view of the implantable delivery device in FIG. 12E according to some embodiments.

FIG. 12G is a plan schematic representation of an implantable delivery device with a multidirectional sprayed dispensing operation according to some embodiments.

FIG. 12H is a top view of the implantable delivery device in FIG. 12G according to some embodiments.

FIG. 12I is a plan schematic representation of an implantable delivery device with a sprayed multidirectional dispensing delivery arm according to some embodiments.

FIG. 12J is a top view of the implantable delivery device in FIG. 12I according to some embodiments.

FIG. 12K is a plan schematic representation of an implantable delivery device with a guided multidirectional dispensing delivery arm according to some embodiments.

FIG. 12L is a top view of the implantable delivery device in FIG. 12K according to some embodiments.

FIG. 12M is a plan schematic representation of an implantable delivery device with a sprayed multidirectional dispensing main portion and delivery arm according to some embodiments.

FIG. 12N is a top view of the implantable delivery device in FIG. 12M according to some embodiments.

FIG. 12O is a plan schematic representation of an implantable delivery device with a guided multidirectional dispensing delivery arm and focused dispensing main portion according to some embodiments.

FIG. 12P is a top view of the implantable delivery device in FIG. 12M according to some embodiments.

FIG. 13A is a schematic view of a medicament delivery system implanted at an anterior reservoir location.

FIG. 13B is a detailed view of the implantation location and arrangement of the implantable delivery device of FIG. 13A.

FIG. 14A is a schematic view of a medicament delivery system implanted at a posterior reservoir location.

FIG. 14B is a detailed view of the implantation location and arrangement of the implantable delivery device of FIG. 14A.

FIG. 15A is a schematic view of a medicament delivery system implanted at an anterior-posterior reservoir location.

FIG. 15B is a detailed view of the implantation location and arrangement of the implantable delivery device of FIG. 15A.

FIG. 16A is a schematic view of a medicament delivery system implanted at an anterior-intravitreal reservoir location.

FIG. 16B is a detailed view of the implantation location and arrangement of the implantable delivery device of FIG. 16A.

FIG. 17A is a schematic view of a medicament delivery system implanted at an anterior-anterior chamber reservoir location.

FIG. 17B is a detailed view of the implantation location and arrangement of the implantable delivery device of FIG. 17A.

FIG. 18 is a microscopic view of a microporous material according to some embodiments that presents microscopic imagery of a material structure represented illustratively in portions of FIGS. 10A and 10C.

FIGS. 19A through 19D are images of the medicament delivery system being implanted in the eye tissue in different configurations according to some embodiments disclosed herein.

### DETAILED DESCRIPTION

Persons skilled in the art will readily appreciate that the various embodiments of the inventive concepts provided in the present disclosure can be realized by any number of methods and apparatuses configured to perform the intended functions. It should also be noted that the accompanying figures referred to herein are not necessarily drawn to scale, but may be exaggerated to illustrate various aspects of the present disclosure, and in that regard, the figures should not be construed as limiting. Some figures do, however, represent anatomy and the positioning of embodiments relative to that anatomy and such representations should be understood to be scaled and positioned accurately, with some deviation permitted as the anatomical structures depicted will vary in size and position from person to person.

The present disclosure relates to systems, devices, and methods for delivering a medicament to an eye of a patient. In various embodiments, the medicament is an ocular medicament that is configured to treat, for example, ocular hypertension and/or glaucoma, by causing the intraocular pressure to decrease from undesirably high levels that may lead to a gradual and sometimes permanent loss of vision in the afflicted eye. In various embodiments, medicament delivery systems according to the instant disclosure are configured to meter drug release rates for one or more different medicaments, and thus may be configured to provide multiple different release rates, including multiple different release rates for multiple medicaments. Some examples of suitable ocular medicaments include therapeutic agents, such as prostaglandin analogs (PGAs) (e.g., latanoprost), or therapeutic agents from other drug classes, including beta-blockers such as timolol, alpha-2-agonists such as brimonidine tartrate, or carbonic anhydrase inhibitors such as dorzolamide, compounds of carbonic anhydrase inhibitors and beta-blockers, and compounds of alpha-agonists and beta-blockers which may be administered in combination with PGAs.

In some embodiments, such medicament delivery systems are configured to be implanted and minimally invasively refillable one or more times in situ without requiring removal of the medicament delivery system from an implantation site. Given the size and subconjunctival target implantation locations, implantation procedures can be performed outside of the operating room, where needle puncture and small incisions are commonly performed. Additionally, some system examples include features for helping reduce micro-movement between the medicament delivery systems and the tissue into which they are implanted. Micro-movement may be defined as small movements between the medicament delivery systems and the tissue, wherein the movements may be on a micrometer or millimeter, and microsecond or millisecond scale. Micro-movement sometimes leads to irritation of the surrounding tissue, which is known to lead to a foreign body tissue response that can cause excessive scar formation, eventual erosion of implanted devices, and/or site infection.

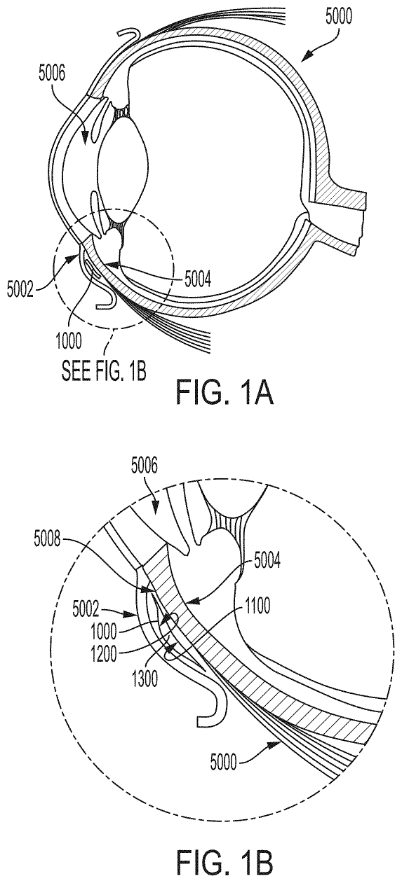

A medicament delivery system 1000 according to some embodiments is illustrated in FIG. 1A. As illustrated, the medicament delivery system 1000 is implanted within the eye 5000 between the conjunctiva 5002 and the sclera 5004 of the eye 5000. Also shown is an anterior chamber 5006. The medicament delivery system 1000 generally includes one or more portions that are configured to meter drug release from the medicament delivery system 1000, as well as one or more portions that are configured to promote or permit cellular infiltration and/or tissue attachment. The medicament may include a single therapeutic agent (e.g., a medication), or may include multiple therapeutic agents. The medicament may include additional materials (e.g. bioabsorbable polymers, pharmaceutically acceptable carrier) to affect the elution of the therapeutic agents (e.g. bioabsorbable polymers) from the delivery system. Throughout the description herein, the medicament may also be referred to as a pharmaceutical composition or combination as is may be composed of both a therapeutic agent and/or additional materials for effective elution of the therapeutic agent. For example, the medicament may include bioabsorbable microparticles having a size ranging between approximately 0.1 microns to 50 microns, or from approximately 1 micron to 50 microns, or from approximately 5 microns to 50 microns, or from approximately 15 microns to 50 microns, or from approximately 10 microns to 40 microns, or approximately from 15 microns to 25 microns, or approximately 18 microns to 23 microns. In some embodiments, the bioabsorbable microparticles have an average size of approximately 20 microns. In further embodiments, the therapeutic agent retained in the bioabsorbable microparticles may be latanoprost. As will be described further herein, the bioabsorbable microparticles may be retained within the medicament delivery system 1000 during use, while the drug may be released from the bioabsorbable microparticles, and as such, the medicament delivery system 1000. The medicament delivery system 1000 may be configured to meter drug release rates for multiple different medicaments at multiple different release rates.

As previously described, the medicament may be a pharmaceutical composition composed of at least one therapeutic agent and at least one additional material, such as, but not limited to, a bioabsorbable microparticle. The pharmaceutical composition is capable of adopting a first state and adopting a second state and is capable of transitioning between the first state and second state. For example, the pharmaceutical composition may transition from the first state to the second state when exposed to a fluid. In certain embodiments, the presence of the fluid may initiate a transition of the composition from the first state to the second state. In some embodiments, the first state corresponds to a non-therapeutic state and the second state correspond to a therapeutic state, such that in the absence of a sufficient amount of fluid, the therapeutic agent may not be released from the composition (and hence not administered to the patient in need thereof). In the therapeutic state, an amount of therapeutic agent released from the additional material may be increased relative to the amount released in the non-therapeutic state. In the therapeutic state, the therapeutic agent may be released in a pharmaceutical effective amount sufficient to treat the patient in need thereof. In some embodiments, the first state is substantially free of fluid.

In certain embodiments, the fluid comprises water. However, in other embodiments, various other fluids may also be present. In some embodiments, the time required for a provided composition to convert into a second state may vary in an application-appropriate manner, and may be relatively short. For example, in some embodiments, the pharmaceutical composition converts to a second state in a period of time between 10 seconds and 1 week after exposure to fluid (e.g., 30 seconds to 6 days, 1 minute to 5 days, 1 minute to 4 days, 1 minute to 3 days, 1 minute to 2 days, or 1 minute to 1 day).

Similarly, the time for a provided composition to remain in a second state may vary in an application-appropriate manner, and may be relatively long. For example, in some embodiments, the pharmaceutical composition may remain in a second state for a period of time between 1 min and 1 year after exposure to fluid and any time frame encompassed therein, such as 1 hour, 1 day, 1 week, 1 month, 2 months, 3 months, 4 months, 5 months, 8 months, or 11 months. Put differently, the quotidian influx of the medicament into the eye of the patient from the reservoir is consistent and controllable over the time period. Without wishing to be bound by any particular theory, this may be accomplished by using pharmaceutically acceptable carrier microparticles of various size and geometric complexities, where the conversion of the pharmaceutical composition occurs at different time points over the few months period based on the pace of degradation of the additional bioabsorbable material.

FIG. 1B is a detail view of region 1B in FIG. 1A, and illustrates the medicament delivery system 1000 implanted within a subconjunctival space 5008. As shown, the subconjunctival space 5008 is a pocket formed between the conjunctiva 5002 and the sclera 5004 of the eye 5000. The subconjunctival space 5008 shown in FIGS. 1A and 1B may be formed according to known methods. In some embodiments, the medicament delivery system 1000 is implantable ab-externally (e.g., from outside of the eye), such as through a conjunctival incision. In some embodiments, a conjunctival radial incision is performed near the limbal junction, and blunt dissection of the conjunctiva is performed to expose the sclera and to form a subconjunctival pocket for placement of the medicament delivery system 1000. In other embodiments, the medicament delivery system 1000 is implanted ab-internally (e.g., from inside the eye), such as through a clear-corneal incision, and placed through the sclera 5004 and into a dissected subconjunctival space 5008.

In some embodiments, the medicament delivery system 1000 may be further secured to the sclera 5004 or other surrounding tissue, such as by way of suturing, adhesives, or according to other known methods. For example, sutures may be used to secure the medicament delivery system 1000 in place onto the sclera 5004. Though the medicament delivery system 1000 may be permanently or semi-permanently secured at the time of implantation, the medicament delivery system 1000 may also initially be temporarily secured (or initially not secured at all), and subsequently secured to the sclera 5004 or other surrounding tissue by one or more portions of medicament delivery system 1000 configured to promote or permit cellular infiltration and tissue attachment.

With continued reference to FIGS. 1A and 1B, the medicament delivery system 1000 includes at least a first stratum 1100 and a second stratum 1200. The first and second strata 1100 and 1200 are generally coupled together in a manner that provides for a medicament reservoir 1300 being defined between the first and second strata 1100 and 1200, as depicted in FIG. 1B. The medicament reservoir 1300 is generally an enclosed spaced within which a medicament can be deposited for subsequent delivery by the medicament delivery system 1000. Generally, the medicament delivery system 1000 is configured to meter a release rate of the medicament disposed within the medicament reservoir 1300 over a designated period of time. For instance, a medicament can be disposed or deposited within the medicament reservoir 1300 and the medicament delivery system 1000 can be configured to release the medicament in accordance with a predetermined therapy regime to treat one or more deficiencies or conditions of the eye.

Moreover, the medicament delivery system 1000 may be minimally invasively refillable and/or emptiable in situ (e.g., without first requiring removal of the medicament delivery system 1000 from the implantation site). In some such embodiments, one or more of the first and second strata 1100 and 1200 are configured such that they may be repeatedly pierced with a cannula during medicament reservoir refilling or emptying operations without significantly jeopardizing the integrity of the first and/or second strata 1100 and 1200. In some embodiments, this integrity may be achieved by coating or imbibing the first and/or second strata 1100 and 1200 with an elastomeric material.

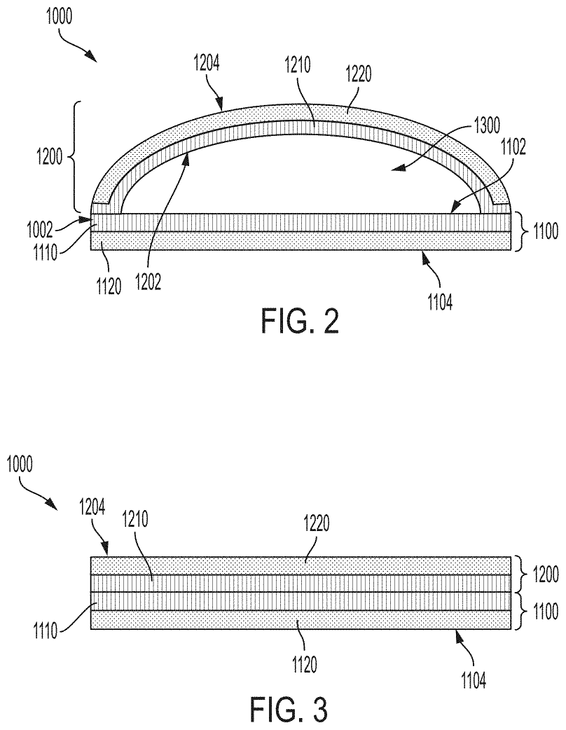

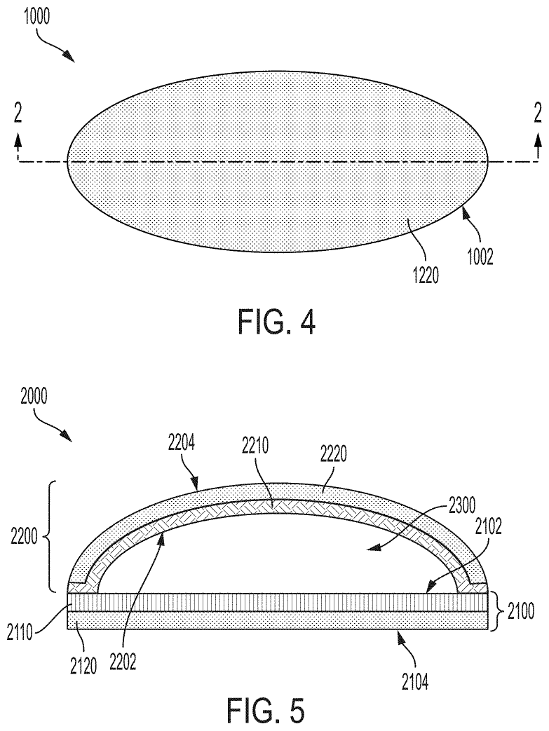

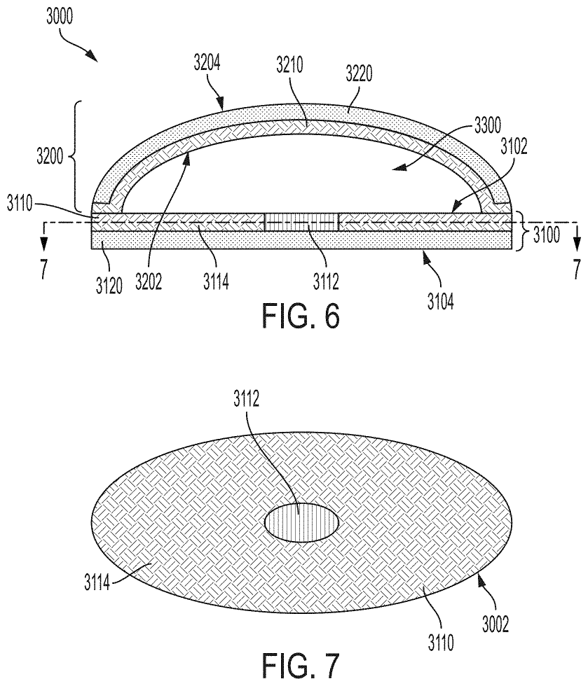

With reference now to FIGS. 2-4, a medicament delivery system 1000 is shown. FIG. 2 is a cross-sectional view of the medicament delivery system 1000 shown in an inflated state (i.e., where the medicament reservoir 1300 is inflated such that a separation is defined between the first and second strata 1100 and 1200), taken along line 2-2 of the medicament delivery system illustrated in FIG. 4. In some embodiments, the inflated state corresponds to the presence of a medicament in the medicament reservoir 1300. FIG. 3 is a cross-sectional view of the medicament delivery system 1000 taken along line 2-2 of the medicament delivery system illustrated in FIG. 4, shown in a deflated state, such as when the medicament reservoir 1300 is empty or devoid of a sufficient amount of medicament to cause a separation between the first and second strata 1100 and 1200. FIG. 4 is a front view of a medicament delivery system 1000.

As depicted in FIG. 2, the first and second strata 1100 and 1200 are coupled together along one or more portions of the first and second strata 1100 and 1200, while one or more other portions of the first and second strata 1100 and 1200 remain uncoupled. The uncoupled portions of the first and second strata 1100 and 1200 remain free to separate from one another. In various embodiments, the uncoupled portions of the first and second strata 1100 and 1200 are operable to separate from one another to define the medicament reservoir 1300.

In some embodiments, the first and second strata 1100 and 1200 may be coupled together about a peripheral edge 1002 of the medicament delivery system 1000, as shown in FIG. 2. However, it is to be appreciated that the first and second strata 1100 and 1200 may additionally or alternatively be coupled in other regions, including one or more regions interior to the peripheral edge 1002. The peripheral edge 1002 is generally an edge that extends about the periphery of the medicament delivery system 1000. The peripheral edge 1002 may be uniform, non-uniform, continuous, or discontinuous. For instance, in some embodiments, the peripheral edge 1002 may include one or more radially extending tabs or petals (e.g., the medicament delivery system 1000 may include a scalloped peripheral edge). In some embodiments, these tabs or petals may operate as a coupling region for coupling the medicament delivery system 1000 to surrounding tissue, such as the sclera 5004.

As shown in FIG. 2, the first and second strata 1100 and 1200 are coupled together at the peripheral edge 1002 and/or along a region just radially inwardly of the peripheral edge 1002 to form a coupling region that extends adjacent to the peripheral edge 1002. The coupling region may be annularly shaped and may extend as radially inwardly from the peripheral edge 1002 as desired. As shown in FIG. 2, the medicament reservoir 1300 is defined between the first and second strata 1100 and 1200 where the first and second strata 1100 and 1200 remain uncoupled. It will be appreciated that the first and second strata 1100 and 1200 may additionally be coupled together at a plurality of discrete locations or regions, including one or more locations or regions radially inwardly of the peripheral edge 1002 of the medicament delivery system 1000. Coupling together one or more additional regions interior to the peripheral edge may help to control an inflation profile of the medicament delivery system 1000.

One or more of the first and second strata 1100 and 1200 may be configured to elastically or plastically deform as the medicament reservoir 1300 is inflated. Moreover, in some embodiments, one the first and second strata 1100 and 1200 may be inelastic, which may help to control an expansion profile of the medicament delivery system 1000.

In various embodiments, one or both of the first and second strata 1100 and 1200 includes one or more regions configured to meter a release of medicament. These metering regions may be in the form of membranes, layers, or films, or coatings. In some embodiments, one or more of the first and second strata 1100 and 1200 includes one or more regions configured to permit or promote cellular infiltration or tissue ingrowth and attachment. Cellular infiltration and tissue attachment generally occurs where materials are of a sufficiently porous nature to permit fibroblastic infiltration. Accordingly, the medicament delivery system 1000 may include membranes, layers, films, and/or coatings that are configured to permit tissue ingrowth and attachment.

In at least one embodiment, the first and/or second strata 1100 and 1200 may be formed of a plurality of membrane layers. For example, as shown in FIGS. 2 and 3, the first stratum 1100 may include a first membrane layer 1110 and a second membrane layer 1120. The first and second membrane layers 1110 and 1120 of the first stratum 1100 collectively define the first stratum 1100. It will be appreciated that the first stratum 1100 may include membrane layers in addition to the first and second membrane layers 1110 and 1120.

In some embodiments, one or more of the first and second membrane layers 1110 and 1120 may include a microporous microstructure. For example, one or more of the first and second membrane layers 1110 and 1120 may include biocompatible materials such as expanded polytetrafluoroethylene (ePTFE). Additionally, one or more of first and second membrane layers 1110 and 1120 of the first stratum 1100 may be formed of other biocompatible materials including biocompatible polymers, which may or may not be microporous, including, but not limited to, polyurethane, silicone, polysulfone, polyvinylidene fluorine (PVDF), polyhexafluoropropylene (PHFP), perfluoroalkoxy polymer (PFA), polyolefin, fluorinated ethylene propylene (FEP), acrylic copolymers and polytetrafluoroethylene (PTFE).

The first and/or second membrane layers may be in the form of one or more sheets or films, and they may include knitted, woven, and/or non-woven forms including individual or multi-fiber strands. In some embodiments, the first and/or second membrane layers 1110 and 1120 may be formed from a plurality of sheets or films of polymer material. In some embodiments, the sheets or films may be laminated or otherwise mechanically coupled together to form the first and/or second membrane layers 1110 and 1120 of the first stratum 1100. Coupling of the sheets or films may be accomplished by a variety of mechanisms, including heat treatment, high pressure compression, bonding agents such as one or more adhesives, lamination, or other suitable methods known to one of skill in the art.

In some embodiments, adjacently-situated membrane layers (e.g., first and second membrane layers 1110 and 1120) and/or the layers of material forming such membrane layers, may be partially or completely bonded via thermal methods where each of the polymers forming the materials are brought to or above their melting temperatures. In some embodiments, such thermal processes facilitate adhesive or cohesive bond formation between the materials or layers of material. In some embodiments, adjacently situated membrane layers and/or the layers of material forming such membrane layers, may be partially or completely bonded via thermal methods where at least one of the materials is brought to or above its melting temperature. Such thermal processes may facilitate adhesive or cohesive bond formation between the materials or layers of material. In some embodiments, one or more suitable adhesives are utilized and provide a sufficiently bonded interface. Adjacently situated membrane layers and/or the layers of material forming such membrane layers may be coupled together at one or more discrete locations to form stabilizing structures that extend through the resulting structure.

In some embodiments, the first stratum 1100, and/or the first and second membrane layers 1110 and 1120, and/or the sheets or films from which the first and second membrane layers 1110 and 1120 are formed may be subjected to one or more processes to modify a microstructure thereof. In some embodiments, such processes include, but are not limited to, material coating processes, surface pre-conditioning processes, and/or perforation processes. Material coating processes may be utilized to apply one or more drug or antimicrobial coatings to the polymer material (such as metallic salts (e.g. silver carbonate) and organic compounds (e.g. chlorhexidine diacetate). Hydrophilic coatings to enable wetout—including immediate wetout—of the polymer matrix can also be applied as polymer surfaces that are generally hydrophobic in nature. Surface coatings including antioxidant components can additionally or alternatively be applied to mitigate the body's inflammatory response that naturally occurs during wound healing after surgery. Material surfaces can additionally or alternatively be modified with anti-proliferative compounds (e.g., Mitomycin C, 5-fluoracil) to moderate the surrounding tissue response.

In some embodiments, one or more surface pre-conditioning processes may be utilized to form layers exhibiting an exemplary microstructure (e.g., wrinkles, folds, or other geometric out-of-plane structures), as explained in U.S. Pat. No. 9,849,629 to Zaggl. Such surface pre-conditioning could facilitate a bolder early inflammatory phase after surgery, providing an early stable interface between porous device and tissue. In some embodiments, a heparin coating may additionally or alternatively be applied to help minimize cell formation including fibrinogen buildup following a surgical implantation procedure.

In some embodiments, one or more perforation processes may be utilized to form a plurality of perforations or pores in one or more of the first and second membrane layers 1110 and 1120 of the first stratum 1100 to achieve a desired porosity. That is, one or more perforation processes may be utilized in addition to a reliance on any interstices, pores (voids between fibril and nodes making up the microstructure), and/or channels naturally occurring within the polymer material.

It will be appreciated that the first and second membrane layers 1110 and 1120 of the first stratum 1100 may be processed differently to achieve membrane layers having different material properties, such as different porosities and/or different cellular infiltration potential. In some embodiments, the first and second membrane layers 1110 and 1120 of the first stratum may not by subjected to any processing steps.

In some embodiments, the first membrane layer 1110 (also referred to herein as a medicament metering membrane layer) is configured to meter a rate at which a medicament passes through the first membrane layer 1110 and thus a rate at which a medicament is released by the medicament delivery system 1000. In various embodiments, the first membrane layer 1110 is also configured to resist cellular infiltration and attachment. In some embodiments, the first membrane layer 1110 includes interstices, perforations, pores, channels, or combinations thereof that are sized and shaped to resist, impede, or otherwise minimize cellular infiltration while remaining permeable to one or more medicaments. The interstices, perforations, pores, or channels of the first membrane layer 1110 of the first stratum 1100 may be less than (or have an average size of less than) about one (1) to about two (2) microns, for example, although a variety of dimensions may be selected based upon application. By being resistant to cellular ingrowth and attachment, the first membrane layer 1110 of the first stratum 1100 operates to maintain a separation between the medicament disposed within the medicament reservoir 1300 and the tissue surrounding the medicament delivery system 1000. This separation operates to maintain a controlled and stable rate at which medicament is released by the medicament delivery system 1000.

The second membrane layer 1120 (also referred to herein as an ingrowth membrane layer) is configured to promote or permit cellular infiltration and attachment. The second membrane layer 1120 thus generally includes interstices, perforations, pores, channels, or combinations thereof that are sized and shaped to promote or permit cellular infiltration. Thus, the second membrane layer 1120 generally includes interstices, perforations, pores, channels or combinations thereof having an average size that exceeds an average size of the interstices, perforations, pores, or channels of the first membrane layer 1110 of the first stratum 1100. In some embodiments, the second membrane layer 1120 may include interstices, perforations, pores, or channels that range in size (or average size) from between twenty (20) microns and one hundred (100) microns, although a variety of dimensions are contemplated. For example, the size (or average size) of the interstices, perforations, pores, or channels may exceed one hundred fifty (150) microns in other embodiments. Thus, while the first membrane layer 1110 operates to meter and maintain a controlled and stable rate at which medicament is released by the medicament delivery system 1000, the second membrane layer 1120 helps facilitate biointegration of the medicament delivery system 1000 by permitting cellular ingrowth and tissue attachment. Cellular ingrowth and tissue attachment helps minimize micro-movement.

In some embodiments, the interface between the first and second membrane layers 1110 and 1120 of the first stratum 1100 operates as a boundary to cellular infiltration into the first membrane layer 1110. That is, in some embodiments, the first stratum 1100 is configured such that cellular infiltration and proliferation is limited to within the second membrane layer 1120, and not into the first membrane layer 1110. Thus, in various embodiments, cellular infiltration, and proliferation within the second membrane layer 1120 can generally propagate up to the boundary between the first and second membrane layers 1110 and 1120. In some embodiments, the first stratum 1100 may be configured to prevent or otherwise minimize a potential for cellular infiltration and proliferation across the boundary between the first and second membrane layers 1110 and 1120 of the first stratum 1100.

It should also be appreciated that while the first stratum 1100 (and the corresponding first and second membrane layers 1110 and 1120 of the first stratum 1100) of the medicament delivery system 1000 shown in the accompanying figures are ovularly shaped, the first and second membrane layers 1110 and 1120 and thus the first stratum 1100 may be formed of other shapes and/or sizes provided that the medicament delivery system 1000 effectively fulfills its intended purpose of being implantable within a tissue, such as a subconjunctival pocket, and operable to cause a release of a medicament disposed within the medicament reservoir 1300 of the medicament delivery system 1000 to one or more regions of the tissue surrounding the medicament delivery system 1000. For instance, the first and second membrane layers 1110 and 1120 and thus the first stratum 1100 may be square, rectangular, trapezoidal, or any other polygonal or non-polygonal shape (e.g., bean-shaped) as desired, provided the shape does not prohibit implantation or render the medicament reservoir 1300 incapable of dispensing medicament.

As shown in FIGS. 2 and 3, the second stratum 1200 of the medicament delivery system 1000 includes a first membrane layer 1210 and a second membrane layer 1220. The first membrane layer 1210 of the second stratum 1200 is similar to the first membrane layer 1110 of the first stratum 1100 in that the first membrane layer 1210 of the second stratum 1200 is configured to meter a rate at which a medicament passes through the first membrane layer 1210 and thus a rate at which a medicament is released by the medicament delivery system 1000. In various embodiments, the first membrane layer 1210 is also configured to resist cellular infiltration and attachment. The first membrane layer 1210 thus generally includes interstices, perforations, pores, channels, or combinations thereof consistent with those discussed above for the first membrane layer 1110 of the first stratum 1100.

The second membrane layer 1220 the second stratum 1200 is similar to the second membrane layer 1120 of the first stratum 1100 in that the second membrane layer 1220 of the second stratum 1200 is configured to promote or permit cellular infiltration and attachment. The second membrane layer 1220 thus generally includes interstices, perforations, pores, channels, or combinations thereof consistent with those discussed above for the second membrane layer 1120 of the first stratum 1100. Thus, in various embodiments, the medicament delivery system 1000 includes a second stratum 1200 that is formed of a first membrane layer 1210 and a second membrane layer 1220 where the first membrane layer 1210 is permeable to a medicament and configured to resist cellular infiltration and tissue attachment and where the second membrane layer 1220 is permeable to the medicament and configured to promote or permit cellular infiltration and tissue attachment.

In some embodiments, the interface between the first and second membrane layers 1210 and 1220 of the second stratum 1200 operates as a boundary to cellular infiltration into the first membrane layer 1210. That is, in some embodiments, the second stratum 1200 is configured such that cellular infiltration and proliferation is limited to the second membrane layer 1220, and not into the first membrane layer 1210. Thus, in various embodiments, cellular infiltration and proliferation within the second membrane layer 1220 can generally propagate up to, but not through, the boundary between the first and second membrane layers 1210 and 1220. In some embodiments, the second stratum 1200 may be configured to prevent or otherwise minimize a potential for cellular infiltration and proliferation across the boundary between the first and second membrane layers 1210 and 1220 of the second stratum 1200. It should be appreciated that the second stratum 1200 may include membrane layers in addition to the first and second membrane layers 1210 and 1220.

Similar to the first stratum 1100 mentioned above, the second stratum 1200 may be formed of shapes and/or sizes other than those depicted in the accompanying figures (e.g., square, rectangular, trapezoidal, bean-shaped, or any other polygonal or non-polygonal shape) provided that the medicament delivery system 1000 effectively fulfills its intended purpose of being implantable within a tissue and operable to cause a release of a medicament disposed within the medicament reservoir 1300 to one or more regions of the tissue surrounding the medicament delivery system 1000.

As shown in FIGS. 2 and 3, the first stratum 1100 is oriented such that the first membrane layer 1110 is situated adjacent the second stratum 1200 (and the first membrane layer 1210 of the second stratum 1200 in particular), and includes a first face 1102 that faces or is otherwise exposed to the second stratum 1200 (and the first membrane layer 1210 of the second stratum 1200 in particular). That is, in some embodiments, the first stratum 1100 is situated such that the first membrane layer 1110 is positioned between the second membrane layer 1120 and the second stratum 1200. Such a configuration provides that the first membrane layer 1110 of the first stratum 1100 at least partially defines the medicament reservoir 1300. That is, in various embodiments, the medicament reservoir 1300 is defined, at least in part, by one or more medicament metering membrane layers (e.g., first membrane layer 1110) that are configured to meter a rate at which a medicament is released from the medicament reservoir 1300. Such a configuration also provides that the second membrane layer 1120 of the first stratum 1100 includes a second face 1104 opposite the first face 1102 and partially defines an exterior of the medicament delivery system 1000. That is, in various embodiments, an exterior of the medicament delivery system 1000 is defined, at least in part, by one or more tissue ingrowth membrane layers (e.g., second membrane layer 1120), which are configured to promote or permit cellular infiltration and tissue attachment, as mentioned above. Promoting or permitting tissue ingrowth and attachment along one or more of the exterior surfaces of the medicament delivery system 1000 helps minimize micro-movement between the medicament delivery system 1000 and the surrounding tissue with which the medicament delivery system 1000 interfaces. Permitting tissue ingrowth and attachment along one of more of the exterior surfaces of the medicament delivery system 1000 further aids in the targeted delivery of the medicament from the device, as it ensures that the medicament is delivered through the desired portion of the device and to the target location within the eye.

Similarly, as shown in FIGS. 2 and 3, the second stratum 1200 is oriented such that the first membrane layer 1210 of the second stratum 1200 is situated adjacent the first stratum 1100 (and the first membrane layer 1110 of the first stratum 1100 in particular), and includes a first face 1202 that faces or is otherwise exposed to the first stratum 1100 (and the first face 1102 of the first membrane layer 1210 of the first stratum 1100 in particular). That is, in some embodiments, the second stratum 1200 is situated such that the first membrane layer 1210 is positioned between the second membrane layer 1220 and the first stratum 1100. Such a configuration provides that the first membrane layer 1210 of the second stratum 1200 at least partially defines the medicament reservoir 1300. Such a configuration also provides that the second membrane layer 1220 of the second stratum 1200 includes a second face 1204 opposite the first face 1202 and partially defines an exterior of the medicament delivery system 1000. Thus, as shown in FIGS. 2 and 3, an exterior of the medicament delivery system 1000 is defined, at least in part, by the second membrane layers 1120 and 1220 of the first and second strata 1100 and 1200, respectively. Additionally, as shown in FIGS. 2 and 3, the medicament reservoir 1300 is defined, in part, by the first membrane layers 1110 and 1210 of the first and second strata 1100 and 1200, respectively. As shown, the medicament reservoir 1300 is defined by those portions of the first membrane layers 1110 and 1210 of the first and second strata 1100 and 1200 that remain uncoupled or that are not otherwise coupled to one another and that are situated radially inwardly of those portions of the first membrane layers 1110 and 1210 of the first and second strata 1100 and 1200 that are coupled together.

In various embodiments, the first and second strata 1100 and 1200 (including the various membrane layers thereof) may be connected or coupled to one another according to known methods, such as by way of heat treatment, high pressure compression, bonding agents such as one or more adhesives, combinations thereof, or other techniques known to those of skill in the art.

In some embodiments, the first faces 1102 and 1202 of the first and second strata 1100 and 1200, respectively, are coupled along the peripheral edge 1002 of the medicament delivery system 1000 such that one or more portions of the first faces 1102 and 1202 of the first and second strata 1100 and 1200 remain uncoupled to one another. In some embodiments, such uncoupled regions remain free to slide, translate, actuate, separate, or otherwise move relative to one another. This relative motion between the uncoupled or unbonded portions of the first and second strata 1100 and 1200 provides that a volume of the medicament reservoir 1300 may vary with an amount of medicament present in the medicament reservoir 1300. For example, the medicament delivery system 1000 may be transitionable between states or configurations, including a first configuration where the medicament reservoir 1300 has a first volume and a second configuration where the medicament reservoir 1300 has a second volume greater than the first volume.

FIG. 3 shows the medicament delivery system 1000 in a first configuration where the medicament reservoir 1300 is deflated (e.g., devoid of or including a negligible amount of medicament), while FIG. 2 shows the medicament delivery system 1000 in a second configuration where the medicament reservoir 1300 is inflated (e.g., full or at least partially filled with medicament). In some embodiments, the medicament delivery system 1000 adopts a relatively flat profile (e.g., a relatively uniform cross section) in a deflated state, in comparison to a profile of the medicament delivery system 1000 in an inflated state. For example, as shown in FIG. 4, the medicament delivery system 1000 may adopt a blister or pillow shape in an inflated state. However, the medicament delivery system 1000 may be configured to adopt any desirable shape or size when devoid of any medicament and/or when filled with medicament.

As the medicament delivery system 1000 is configured to deliver medicament, and because the medicament delivery system 1000 can be refilled or emptied in situ, it will be appreciated that the medicament delivery system 1000 is transitionable between the first and second configurations in situ.

Moreover, the medicament reservoir 1300 may be accessed in situ by way of a cannula, needle or other suitable instrument or method, to add or remove medicament from the medicament reservoir 1300.

The medicament delivery system 1000 shown in FIG. 2 is configured such that medicament is meterable and dispensable from the medicament delivery system 1000 through both the first and the second strata 1100 and 1200. That is, in some embodiments, the medicament delivery system 1000 includes a first and second strata 1100 and 1200 that are permeable to a medicament. In particular, in some embodiments, medicament is released from the medicament delivery system 1000 by passing through the first membrane layers 1110 and 1210 of the first and second strata 1100 and 1200, respectively, and through the second membrane layers 1120 and 1220 of the first and second strata 1100 and 1200, respectively.

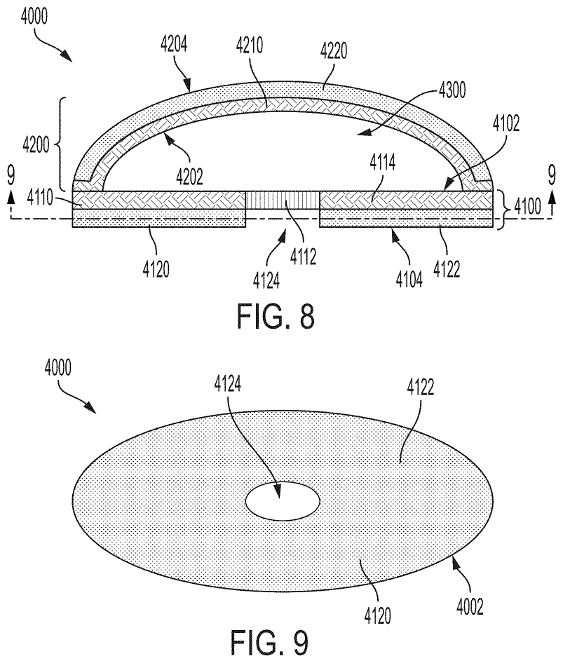

However, in other embodiments, the medicament delivery system may be configured such that medicament is metered and dispensed from the medicament delivery system through one or the other of the first and second strata, but not both. That is, in some embodiments, the medicament delivery system may be configured such that a first one of the first and second strata is permeable to a medicament, while the other of the first and second strata is impermeable to the medicament. For example, turning now to FIG. 5, a medicament delivery system 2000 is shown and includes a medicament permeable first stratum 2100 and a medicament impermeable second stratum 2200. The first stratum 2100 of the medicament delivery system 2000 shown in FIG. 5 is similar to the first stratum 1100 shown in FIGS. 2-4, in that it includes a first membrane layer 2110 (similar to first membrane later 1110) and a second membrane layer 2120 (similar to second membrane later 1120), where the first membrane layer 2110 is permeable to the medicament and is configured to meter a release rate of the medicament disposed within the medicament reservoir 2300 over a designated period of time and is configured to resist cellular infiltration and tissue attachment, and where the second membrane layer 2120 is permeable to the medicament and configured to promote or permit cellular infiltration and tissue attachment. Like the first stratum 1100 of the medicament delivery system 1000 referred to above, the first stratum 2100 of the medicament delivery system 2000 includes a first face 2102 and a second face 2104.

The second stratum 2200 of the medicament delivery system 2000 shown in FIG. 5 includes a first face 2202 and a second face 2204, and is formed of a first membrane layer 2210 and a second membrane layer 2220, where the first membrane layer 2210 is impermeable to medicament. The second membrane layer 2220 of the medicament delivery system 2000 shown in FIG. 5 is similar to the second membrane layer 1220 shown in FIGS. 2-4 in that the second membrane layer 2220 shown in FIG. 5 is configured to promote or permit cellular infiltration and tissue attachment. However, unlike previous examples, the first membrane layer 2210 is impermeable to the medicament disposed within the medicament reservoir 2300. Alternatively, the medicament delivery system 2000 may be configured such that the first stratum 2100 is medicament impermeable while the second stratum 2200 is medicament permeable. The medicament permeable stratum (e.g., first or second strata 2100 or 2200) may include a first medicament permeable membrane layer that is configured to resist cellular infiltration and tissue attachment and/or a second medicament permeable membrane layer that is configured to promote or permit cellular infiltration and tissue attachment. In turn, the medicament impermeable stratum (e.g., first or second strata 2100 or 2200) may include a first medicament impermeable membrane layer that is configured to resist cellular infiltration and tissue attachment and/or a second medicament impermeable membrane layer that is configured to promote or permit cellular infiltration and tissue attachment.

With continued reference to FIG. 5, the second membrane layer 2220 may be formed of any biocompatible material discussed herein, such as a biocompatible polymer, which is further combined with an elastomer or elastomeric material to form a composite material that is impermeable to the medicament. For instance, the second membrane layer 2220 may include a composite material that includes a microporous polymeric membrane having nodes and fibrils where pores are the spaces within the matrix of fibrils (e.g., ePTFE) and a sealing material, such as an elastomeric material, present therein. In some embodiments, the sealing material may be imbibed into the polymeric membrane to form a medicament impermeable membrane layer. It should be appreciated that multiple types of fluoropolymer (and non-fluoropolymer) membranes and multiple types of elastomeric materials can be combined to form a composite material while remaining within the scope of the present disclosure. It should also be appreciated that the elastomeric material can include multiple elastomers as well as multiple types of non-elastomeric components, such as inorganic fillers, therapeutic agents, radiopaque markers, and the like while remaining within the scope of the present disclosure.

In some embodiments, the various membrane layers are formed of expanded polytetrafluoroethylene (ePTFE), although other biocompatible polymers suitable for use in forming medicament impermeable membrane layers including, but not limited to, urethanes, silicones (organopolysiloxanes), copolymers of silicon-urethane, styrene/isobutylene copolymers, polyisobutylene, polyethylene-co-poly(vinyl acetate), polyester copolymers, nylon copolymers, fluorinated hydrocarbon polymers and copolymers or mixtures of any of the foregoing may be used.

In various embodiments, the elastomer or elastomeric material may include perfluoromethyl vinyl ether and tetrafluoroethylene, (per)fluoroalkylvinylethers (PAVE), a copolymer of tetrafluoroethylene and perfluoromethyl vinyl ether, silicone, a fluoroelastomer, a urethane, or a TFE/PMVE copolymer.

With continued reference to the medicament delivery system 2000 shown in FIG. 5, by including a medicament permeable first stratum 2100 and a medicament impermeable second stratum 2200, the medicament delivery system 2000 can be configured to unidirectionally meter and dispense the medicament disposed within the medicament reservoir 2300. That is, in some embodiments, the medicament delivery system 2000 can be configured such that medicament is metered and released through one of the first and second strata 2100 and 2200, but not through the other of the first and second strata 2100 and 2200. Thus, in these embodiments, the medicament delivery system 2000 can be configured such that the medicament is released in a first direction (e.g., through the first stratum 2100) without also releasing the medicament in a second direction (e.g., through the second stratum 2200). Providing this type of controlled release helps provide for directing the dispensing of medicament to a designated tissue. For example, medicament can be released in a direction toward scleral tissue, while minimizing the release of medicament in a direction toward conjunctival tissue, which may be useful is treating conditions within an interior of the eye. Alternatively, medicament can be released in a direction toward conjunctival tissue, while minimizing the release of medicament in a direction toward scleral tissue, which may be useful in treating other conditions of the eye, such as conditions affecting the exterior of the eye (e.g., dry eye). Releasing medicament in a direction toward conjunctival tissue may be used for treating other areas or regions of the body as medicament dispensed to the conjunctiva may be absorbed by the surrounding vasculature and transported to other regions of a patient's anatomy. It should be appreciated that the terms first and second, as used herein with regard to the first and second strata 2100 and 2200, are generic identifiers and thus the strata 2100 and 2200 may be referred to in conjunction with alternative generic identifiers such as top, bottom, upper, lower, side, and so forth. Accordingly, although the stratum 2100 is referred to above in conjunction with the term “first,” and the stratum 2200 is referred to above in conjunction with the term “second,” it should be appreciated that the strata 2100 and 2200 may alternatively be referred to as the first stratum 2200 and the second stratum 2100. That is, the terms “first” and “second” should not be understood to represent anything more than generic identifiers for strata 2100 and 2200.

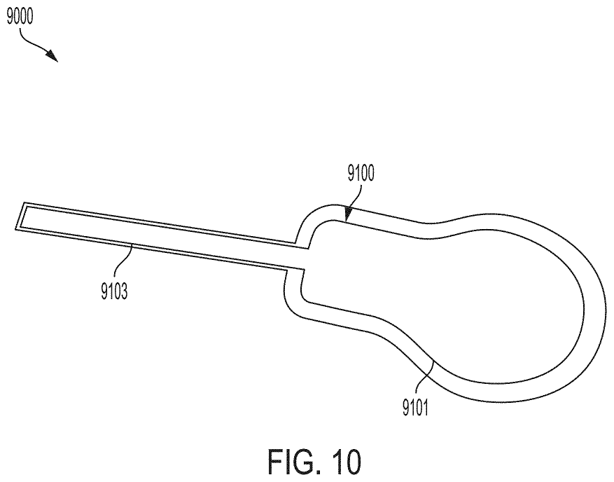

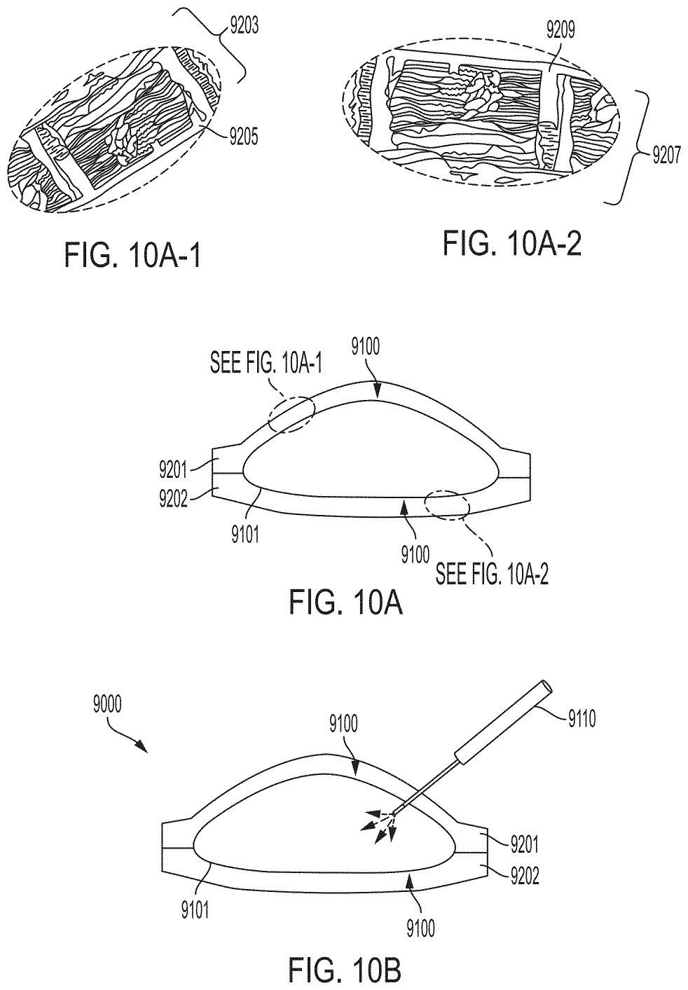

In some embodiments, one or more of the first and second strata may be configured to include one or more medicament permeable portions and one or more medicament impermeable portions. Turning now to FIGS. 6 and 7, a medicament delivery system 3000 is shown, and includes a first stratum 3100 and a second stratum 3200. The second stratum 3200 is consistent in form and construction with the second stratum 2200 of the medicament delivery system 2000 illustrated in FIG. 5 and described above, and includes a first membrane layer 3210, a second membrane layer 3220, a first face 3202, and a second face 3204. The first stratum 3100, on the other hand, is different from previous first stratum examples in that the first stratum 3100 shown in FIGS. 6 and 7 includes a first membrane layer 3110 having a first portion 3112 that is permeable to the medicament and a second portion 3114 that is impermeable to medicament. The first membrane layer 3110 is configured to meter a release rate of the medicament disposed within the medicament reservoir 1300 over a designated period of time. The second membrane layer 3120 is similar to the second membrane layer 3120 of the medicament delivery system 1000 shown in FIG. 5 discussed above.

In various embodiments, the first portion 3112 of the first membrane layer 3110 generally includes interstices, perforations, pores, channels, or other release features that are sized and shaped to allow medicament disposed within the medicament reservoir 3300 to be released through the first portion 3112. In some embodiments, metering the release of the medicament disposed within the medicament reservoir 3300 by the first stratum 3100 may be tuned or otherwise controlled by increasing (or alternatively decreasing) a surface area of the metering first portion 3112 of the first membrane layer 3110. In some embodiments, increasing a surface area of the metering first portion 3112 from a first surface area to a second larger surface area is associated with an increase of an amount of medicament released by the medicament delivery system 3000 per unit of time. Similarly, decreasing a surface area of the metering first portion 3112 from a first surface area to a second smaller surface area is associated with a decrease of an amount of medicament released by the medicament delivery system 3000 per unit of time.

For example, if a medicament delivery system includes a medicament reservoir having a first size (e.g., volume) and a first medicament metering membrane layer having a first surface area and including a first material having a first release rate per unit area, then the medicament delivery system is associated with a first medicament release rate per unit time and with metering a release of the medicament for a first period of time. If the size of the medicament reservoir is increased from the first size to a second, larger size while maintaining the first surface area and the first material of the metering membrane layer, then it should be appreciated that the medicament delivery system is operable to meter a release of medicament for a second period of time longer than the first period of time. On the other hand, if the first surface area of the metering membrane layer is decreased to a second, decreased surface area while the first material of the metering membrane layer and the first size of the medicament reservoir are maintained, then it should be appreciated that the medicament delivery system is operable to meter a release of medicament for a third period of time longer than the first period of time. Additionally, if the first material of the metering membrane layer is changed to a second material which has a second, decreased release rate per unit area, while the first size of the medicament reservoir and the first surface area of the metering membrane layer are maintained, then it should be appreciated that the medicament delivery system is operable to meter a release of medicament for a fourth period of time longer than the first period of time. Combinations of the above concepts may be utilized to maintain a medicament release period while increasing an amount of medicament released per unit of time. For example, if the size of the medicament reservoir is increased from the first size to a second, larger size in combination with an increase in the surface area of the metering membrane layer from the first surface area to a second, increased surface area while maintaining the first material of the metering membrane layer, then it should be appreciated that the medicament delivery system is operable to increase an amount of medicament released during the first period of time.

It is to also be appreciated that different materials may possess different flow rates per unit area, based for example of differing microstructures (e.g., increased quantity and/or size of interstices, perforations, pores, channels or other release features present in the microstructure). Thus, different materials may be additionally or alternatively selected to tune or otherwise control the degree or amount by which the release of the medicament disposed within the medicament reservoir is metered.

The various medicament delivery systems discussed herein thus provide for configurations including a relatively large medicament reservoir without also inherently possessing a high release rate of medicament due to an associated large medicament metering surface area, or alternatively, a relatively small medicament reservoir without also inherently possessing a low release rate of medicament due to an associated low medicament metering surface area. A relatively large medicament reservoir in combination with a low release rate provides for a medicament delivery system 1000 that can be implanted for long periods of time (e.g., weeks, months, a year, or more) without requiring interventions to refill medicament. Conversely, a relatively small medicament reservoir in combination with a high release rate provides for a medicament delivery system that can be implanted and dispense medicament at a fast rate without being oversized and interfering with normal eye operation (e.g., blinking and eye movements).

In addition, it should be appreciated that medicaments having coarser molecular structures generally require that the medicament delivery system include a microstructure having interstices, pores, channels and/or other release features that correspond in size such that the medicament can pass through the material of the medicament metering membrane layer. Thus, it will be appreciated that different medicament delivery systems may be selected for use in administering different medicaments.

With continued reference to the medicament delivery system 3000 shown in FIGS. 6 and 7, the first membrane layer 3110 of the first stratum 3100 may be formed of one or more sheets or films of material, such as any biocompatible material discussed herein), but where the one or more sheets or films of material have been further combined with a sealing material, such as an elastomer or elastomeric material, in the medicament impermeable second portions 3114. Thus, in some embodiments, the medicament impermeable second portions 3114 of the first membrane layer 3110 of the first stratum 3100 may include a composite structure. In some embodiments, the medicament impermeable second portions 3114 of the first membrane layer 3110 of the first stratum 3100 may correspond with portions of the first membrane layer 3110 of the first stratum 3100 that have been selectively imbibed with and/or coated with a sealing material. That is, in various embodiments, first membrane layer 3110 of the first stratum 3100 may be configured such that one or more portions of the first membrane layer 3110 include a sealing material and where one or more other portions of the first membrane layer 3110 remain free of a sealing material, where those portions of the first membrane layer 3110 that include the sealing material correspond with the medicament impermeable second portions 3114, and where those portions of the first membrane layer 3110 that do not include the sealing material correspond with the medicament metering first portions 3112.

While the medicament delivery system 3000 shown in FIGS. 6 and 7 includes an ovulary shaped medicament metering first portion 3112, the medicament metering first portion 3112 may be formed of other shapes and/or sizes other than those depicted in the accompanying figures (e.g., square, rectangular, trapezoidal, bean-shaped, or any other polygonal or non-polygonal shape) provided that the medicament metering first portion 3112 effectively fulfills its intended purpose of metering a release of the medicament. Thus, it is to be appreciated that a boundary defined between the medicament metering first portion 3112 and the medicament impermeable second portion 3114 may be define any suitable shape consistent with the above.

It should also be appreciated that while the medicament delivery system 3000 shown in FIG. 7 includes only a single, centrally located medicament metering first portion 3112, the first membrane layer 3110 of the first stratum 3100 may include a plurality of discrete medicament metering first portions 3112. Similarly, it should be appreciated that the medicament metering first portion 3112 of the first membrane layer 3110 of the first stratum 3100 need not be centrally located, but may instead be positioned at a location offset from a central position.

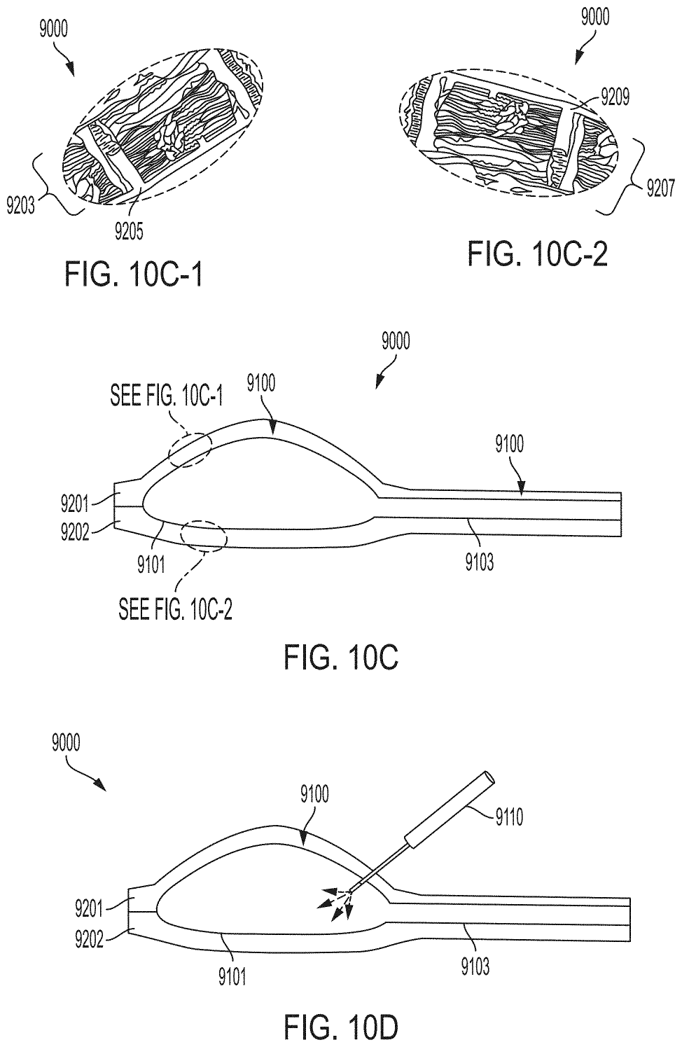

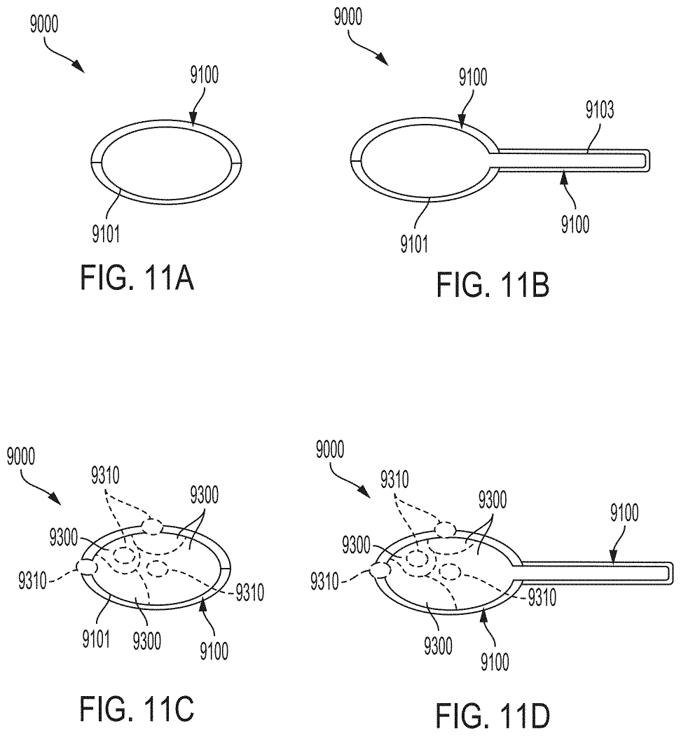

In various embodiments, a medicament delivery system may be configured such that one or more of the medicament metering membrane layers discussed herein may be exposed to a tissue surface of the patient's anatomy. For example, turning now to FIGS. 8 and 9, a medicament delivery system 4000 is shown, and includes a first stratum 4100 and a second stratum 4200. The second stratum 4200 is similar to the second stratum 2200 of the medicament delivery system 2000 shown in FIG. 5 and discussed above, in that the second stratum 4200 is impermeable to the medicament and includes a medicament impermeable first membrane layer 4210 and a second membrane layer 4220 that is configured to promote or permit cellular infiltration and tissue attachment. Similar to the medicament delivery system 2000 shown in FIG. 5 the second stratum 4200 includes a first face 4202 and a second face 4204.

The first stratum 4100 shown in FIGS. 8 and 9 differs, however, from the previous first stratum examples in that the first stratum 4100 shown in FIGS. 8 and 9 includes an aperture or relief which exposes a portion of the first membrane layer 4110. First membrane layer 4110 is configured to meter a release rate of the medicament disposed within the medicament reservoir 4300 over a designated period of time. In particular, as shown in FIGS. 8 and 9, the second membrane layer 4120 includes an aperture 4124 formed in a body 4122 of the second membrane layer 4120, where the aperture 4124 in the second membrane layer 4120 of the first stratum 4100 operates to expose the first membrane layer 4110. When implanted, one or more portions of the first membrane layer 4110 of the first stratum 4100 are directly exposed to tissue, while the second membrane layer 4120 operates to maintain a separation between the first membrane layer 4110 and the tissue surface.

Direct exposure of the first membrane layer 4110 of the first stratum 4100 helps enable more effective and efficient drug delivery to tissue. Maintaining a separation between the first membrane layer 4110 and a tissue surface helps minimize micro-movement between the first membrane layer 4110 and the tissue. As discussed above, minimizing micro-movement helps minimize micro-irritations.

Thus, in various embodiments, the medicament delivery system 4000 includes a medicament permeable first stratum 4100 and a medicament impermeable second stratum 4200, where the first stratum 4100 includes a first membrane layer 4110 and a second membrane layer 4120 and where the first membrane layer 4110 is permeable to a medicament and configured to resist cellular infiltration and tissue attachment and where the second membrane layer 4120 is permeable to the medicament and configured to promote or permit cellular infiltration and tissue attachment and where the second membrane layer 4120 is configured such that one or more portions of the first membrane layer 4110 are exposed to tissue when implanted while the second membrane layer 4120 is positioned between the tissue and one or more other portions of the first membrane layer 4110 while implanted.

Discussion now turns to various implementations as it pertains to treating certain diseases and corresponding configurations of the embodiments disclosed herein. As set forth below, several configurations of implantable medical systems 9000 (e.g., implantable medical system 9000 below), such as a medicament delivery system, are disclosed along with examples of diseases to be treated and corresponding implantation and device locations of the medicament delivery systems. These implantable medical systems 9000 can be similar to others disclosed elsewhere herein, including the medicament delivery systems 1000, 2000, 3000. In this regard, an implantable medical system 9000 can be implanted and arranged at one or more implantations locations or sites to deliver medicament for treatment of a given disease. In addition to glaucoma, diseases for which the medicament delivery system can be useful for treatment include macular degeneration, macular edema, uveitis, retinitis, keratitis, retinoblastoma, retinal vein occlusion (“RVO” such as central RVO (“CRVO”) and branch RVO (“BRVO”)), dry eye, and presbyopia. These diseases can be treated using various active pharmaceutical ingredients (APIs) from a variety of API classes as further discussed below. It should be appreciated, however, that the example implementations discussed herein are just some of many example implementations of the medicament delivery systems disclosed herein and that additional diseases can be treated with such implementations without departing from the scope of this disclosure. More details about these implementations are discussed below.

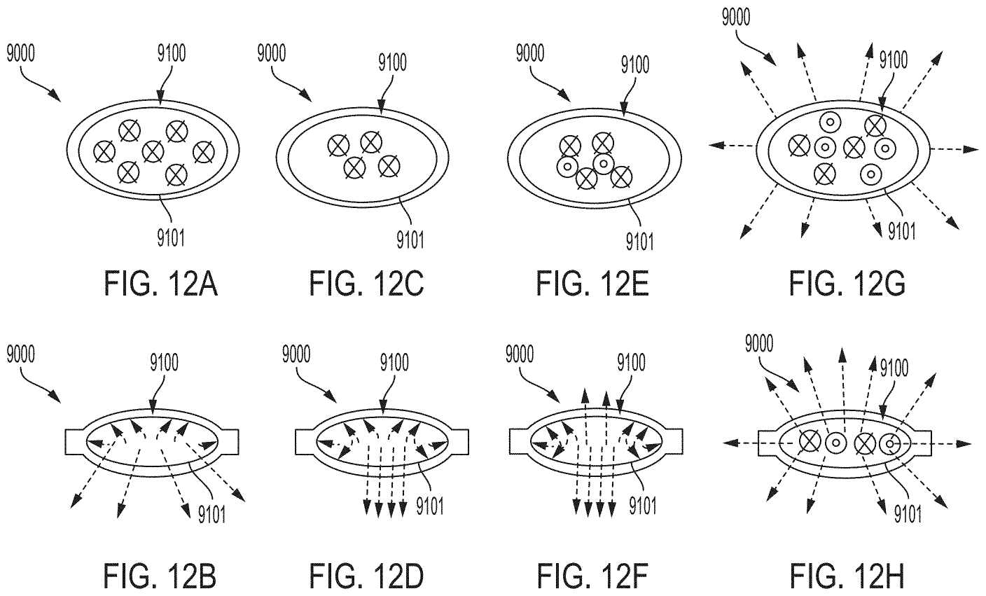

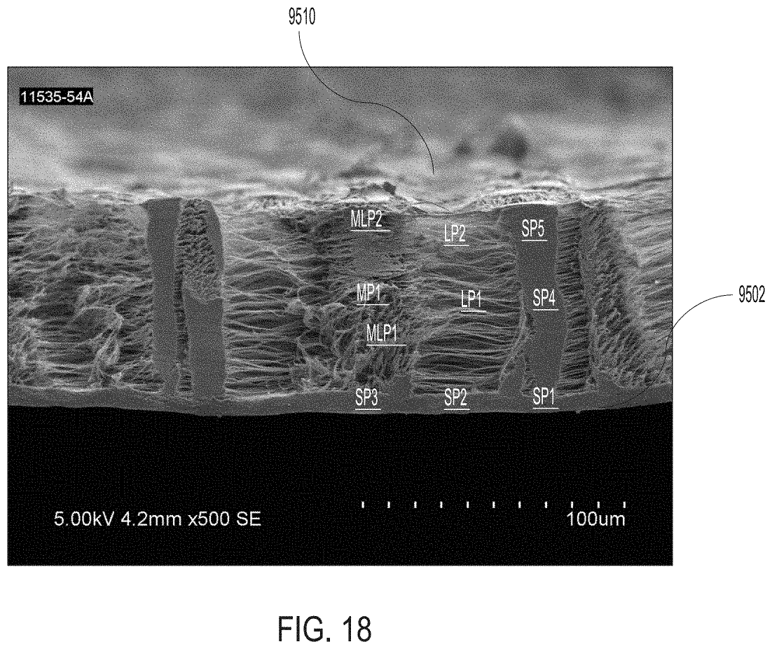

FIGS. 10 and 10A-10D show several examples of an implantable medical systems 9000 that is useful for employing these aspects of the present disclosure. In particular, FIG. 10 is an isometric view of an implantable medical system 9000 having a reservoir 9100 with a main portion 9101 and a delivery arm 9103. While some examples of the implantable deliver devices disclosed herein have delivery arms, as discussed below, some do not have delivery arms. FIG. 10A shows an inflated reservoir 9100. FIGS. 10A-1 and 10A-2 show a microscopic view of a first and second microporous material 9202 respectively in the implantable medical system 9000. FIG. 10B shows the reservoir 9100 in FIG. 10A being refilled via insertion of a syringe 9110 (although other suitable means for refilling the reservoir 9100 in situ may be used). In particular, FIG. 10C shows an inflated reservoir 9100 with a delivery arm 9103. FIGS. 10C-1 and 10C-2 show a microscopic view of a first and second microporous material 9202 respectively in the implantable medical system 9000. FIG. 10D shows the reservoir 9100 in FIG. 10C being refilled via insertion of a syringe 9110 (although other suitable means for refilling the reservoir 9100 in situ may be used). In examples, the main portion 9101 and the delivery arm 9103 can comprise the same or similar materials. Under these circumstances, the delivery arm 9103 can be made integral with or attachable to the main portion 9101 such that the delivery arm 9103 and the main portion 9101 are in fluid communication with each other. The example microporous material is shown as a schematic in FIGS. 10A-1 and 10A-2 for clarity and illustration purposes. The illustrative imagery provided in FIGS. 10A-1, 10A-2, 10C-1 and 10C-2 are representative of the microscopic imagery presented in FIG. 18. Further discussion of microporous materials that are appropriate for employing principles of the present disclosure are shown and described in U.S. Pat. Pub. US2018/0263817, the entire contents of which are hereby incorporated by reference.

The medicant delivery device shown here in FIGS. 10A-10D can be similar to the implantable delivery devices 9000 discussed above. For instance, this device can be used to dispense medicament as discussed above. The implantable medical system 9000 can include the material structure presented in FIGS. 10A-1, 10A-2, 10C-1 and 10C-2 and shown in FIG. 18. The implantable medical system 9000 can also include the material structure, selective permeability, and porous materials shown and described in U.S. Pat. No. 10,849,731 to Cully and as shown and described in U.S. Pat. Publ. No. 2018/0126134 to Cully, both of which are incorporated by reference in their entireties, with particular reference to FIGS. 3-10 and the accompanying descriptions provided in each of those incorporated references. The implantable medical system 9000 can further include a first microporous material 9201 that is bonded to a second microporous material 9202. The first microporous material 9201 can have a first microporous layer 9203 that includes a plurality of pores sized to permit tissue ingrowth. The first microporous material 9201 can have a second microporous layer 9205 that includes a plurality of pores sized to permit tissue ingrowth. The second microporous material 9202 can have a third microporous layer 9205 that includes a plurality of pores sized to resist tissue ingrowth. The second microporous material 9202 can have a fourth microporous layer 9207 that includes a plurality of pores sized to permit tissue ingrowth. The second microporous layer 9205 can be bonded to the third microporous layer 9205 to thereby form a reservoir 9100 for receiving the medicament. The first and second microporous materials 9201 and 9202 can be configured to meter a rate at which the medicament is dispensed from the reservoir 9100 when the delivery device is implanted. While largely discussed herein as receiving a single medicament, in some embodiments, an impermeable material is included within the reservoir 9100 to create separate or distinct chambers or sub-reservoirs that are not in fluid communication with the device 9000. In this way, the reservoir 9100 may accommodate multiple medicaments that may be selectively delivered to the patient. These embodiments may increase the customizability of the device 9000 and increase and/or broaden out which treatments may be used with the device 9000.

According to principles of the present disclosure, the implantable medical system 9000 can be used to treat a variety of diseases as further discussed below. In general, methods of treating diseases using an implantable medical system 9000 configured to meter a rate at which the medicament is dispensed from the reservoir 9100 when the delivery device is implanted are disclosed. The method can include selecting an implantable medical system 9000 that is similar to the implantable medical system 9000 discussed above. For instance, the first microporous material 9201 can have a first microporous layer 9203 that includes a plurality of pores sized to resist tissue ingrowth. The first microporous material 9201 can have a second microporous layer 9205 that includes a plurality of pores sized to permit tissue ingrowth. The second microporous material 9202 can have a third microporous layer 9205 that includes a plurality of pores sized to resist tissue ingrowth. The second microporous material 9202 can have a fourth microporous layer 9207 that includes a plurality of pores sized to permit tissue ingrowth. The second microporous layer 9205 can be bonded to the third microporous layer 9205 to thereby form a reservoir 9100 for receiving the medicament. The first and second microporous materials 9201 and 9202 can be configured to meter a rate at which the medicament is dispensed from the reservoir 9100 when the delivery device is implanted.

Administration of the medicament can include several steps. For instance, the method can include filling the reservoir 9100 with a medicament for treating one or more diseases of the eye. In certain instances, portions or all of the first and second microporous material 9202 can be elastomeric such that they reseal after insertion of a syringe 9110 or other similar devices. The method can include implanting the implantable medical system 9000 at an implantation location. The method can include allowing the medicament to dispense from the reservoir 9100 to one or more treatment sites.

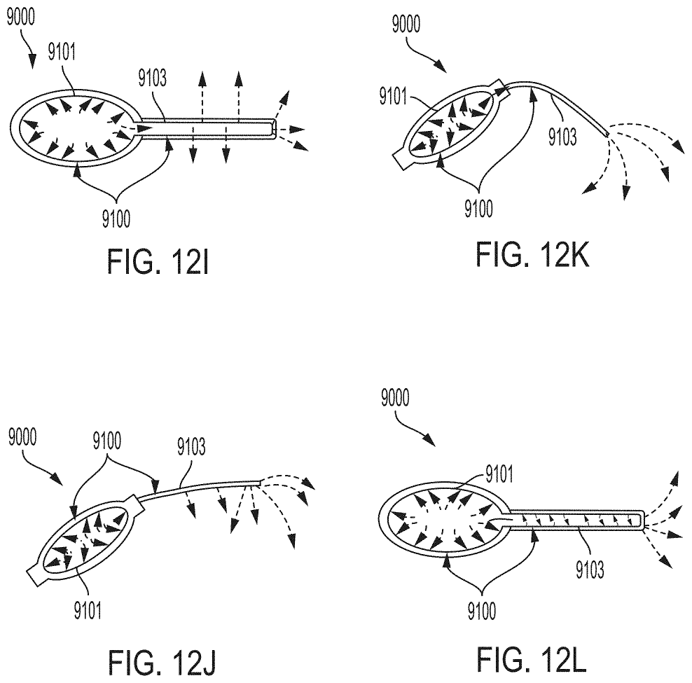

FIGS. 11A-11D show various features of the reservoir 9100 in examples of medicament treatment devices. In particular, FIG. 11A shows a first configuration (Configuration “A”) of the reservoir 9100 where the reservoir 9100 includes a main portion 9101 only. FIG. 11B shows a second configuration (Configuration “B”) of the reservoir 9100 where the reservoir 9100 includes a main portion 9101 and a delivery arm 9103. FIG. 11C shows a third configuration (Configuration “C”) of the reservoir 9100 that is similar to that of FIG. 11A and further includes a plurality of chambers 9300 and a plurality of fill ports 9310 with which to fill a corresponding chamber with medicament. FIG. 11D shows a fourth configuration (Configuration “D”) of the reservoir 9100 that is similar to that of FIG. 11B and further includes a plurality of chambers 9300 and a plurality of fill ports 9310 with which to fill a corresponding chamber with medicament. In examples, fill ports 9310 can be an injection site at the reservoir 9100 or a feature of the implantable medical system 9000 that facilitates filling the reservoir 9100 with medicament. In some embodiments, one of more of the fill ports 9310 is made of transparent, semitransparent, translucid, or translucent material. In this way, the device and/or accessory used to refill the reservoir 9100 or reservoir chambers may be observed when the device and/or accessory is inserted into the fill ports 9310. As further discussed below, the plurality of chambers 9300 (e.g., in FIGS. 11C and 11D) can be in fluid communication or fluidically isolated from one another. In examples, as further discussed elsewhere herein, the reservoir 9100 can dispense medicament in one direction (e.g., into the page) or in multiple directions (e.g., into the page, out of the page, in-plane with the page, or any combination thereof).

A number of factors can influence physical characteristics of medicament treatment devices. Depending on at least one of the implantation location and the disease to be treated, one or both of a geometry and functionality of the reservoir 9100 of the medicament delivery system 1000 can vary in examples. For instance, dimensions the reservoir 9100 can be made larger or smaller when compared to other configurations of the medicament delivery system 1000. These variations can depend on, among other things, the amount of medicament to be delivered for treatment, the manner in which the medicament is to be delivered, and the resistance to delivery of medicament presented by an implantation location. As discussed here, in examples, the reservoir 9100 can have a generally uniform shape (e.g., resembling an ellipse, a circle, a polygon, etc.). In other examples, the reservoir 9100 can have an irregular shape (e.g., a generally uniform shape with one or more extrusions or protrusions therefrom, an eccentric shape, etc.). Some example shapes of the reservoir 9100 are discussed below with respect to certain example implementations of the medicament delivery system 1000. In examples, when the reservoir 9100 is inflated, the reservoir 9100 can bulge about along a length of the reservoir 9100. In some instances, bulging may cause the reservoir 9100 to balloon or to inflate to a generally polyhedron shape.

In certain examples, the physical characteristics of the reservoir 9100 can facilitate functionality of the medicament delivery system 1000. For instance, the reservoir 9100 can be formed to deliver medicament to a pinpoint location relative to the reservoir 9100, generally disperse medicament over a treatment site at one or more sides of the medicament delivery system 1000, and the like. In this regard, it can be said that the implantable medical system 9000 can have one-directional dispensing (e.g., at one side of the implantable medical system 9000) or multidirectional dispensing (e.g., at multiple sides of the implantable medical system 9000). In examples, one or more locations at the reservoir 9100 can function as a fill port 9310 with which to fill the reservoir 9100 with medicament. In this regard, the implantable medical system 9000 can be refillable with medicament as further discussed above. For example, a main portion 9101 of the reservoir 9100 can function as a refillable chamber, and the delivery arm 9103 can function as a conduit with which to deliver fluid from the refillable chamber. In some embodiments, the delivery arm 9103 may be flexible such that it does not kink or compress during use. After implantation, medicament can be flushed or drained from the fill port 9310 at the reservoir 9100 to a treatment location. In examples, the reservoir 9100 can include a plurality of chambers 9300 therein with which to store medicament to be delivered. Under these circumstances, one chamber in the plurality of chambers 9300 can include a first medicament, and another chamber in the plurality of chambers 9300 can include a second medicament that is the same or different from the first medicament. chambers 9300 in the plurality of chambers 9300 can be in fluid communication with one another or isolated from one another depending on the implementation.

Formed at the periphery of the reservoir 9100 can be an extended delivery arm 9103 through which medicament from the reservoir 9100 can be administered. In this regard, the reservoir 9100 can include a reservoir main portion 9101 and the extended delivery arm 9103 extending from the reservoir main portion 9101. For instance, the extended delivery arm 9103 can include a port through which medicament is exclusively allowed to exit the reservoir 9100. In other instances, medicament can be allowed exit the reservoir 9100 at any portion (e.g., not exclusively at the port) of the extended delivery arm 9103. In addition, or in alternative, medicament can be allowed to exit the reservoir 9100 both at the reservoir main portion 9101 and the extended delivery arm 9103.

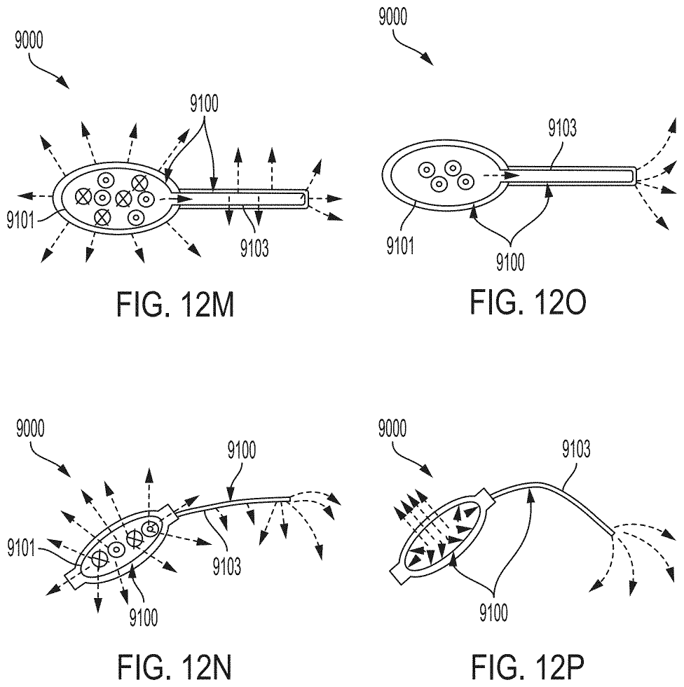

FIGS. 12A-12P show various dispensing configurations of the reservoir 9100. In particular, FIGS. 12A-12H show the implantable medical system 9000 dispensing when in Configuration A of the reservoir 9100 where the reservoir 9100 includes a main portion 9101 only. FIGS. 12I-12P shows a Configuration B of the reservoir 9100 where the reservoir 9100 includes a main portion 9101 and a delivery arm 9103. Although for conciseness, the dispensing operations here are shown with respect to Configurations A and B, one skilled in the art will appreciate that these dispensing operations are similarly applicable to Configurations C and D. In this regard, the distinction would be that Configurations C and D include multiple chambers that may be similarly dispensed to the single chambers discussed below with respect to Configurations A and B. For illustration purposes, dispensing directions are shown as straight arrows flowing out of the reservoir 9100, and dispensing preventions are shown as curly arrows at the interior of the reservoir 9100. Dispensing can occur at only portions of the implantable medical system 9000 that are permeable, and dispensing preventions can occur at impermeable portions of the implantable medical system 9000. In addition, flow and directions into the page are shown as an encircled X, and directions out of the page are shown as encircled dots. Of course, these are merely schematic representations of a more complex phenomena but are used here for clarity of discussion.

As will be discussed below in further detail, dispensing of the medicament can vary across configurations and within configurations. For instance, as discussed above, the implantable medical system 9000 can be configured such that medicament is generally dispensed in one direction or in a plurality of directions. Under these circumstances, dispensing can occur at the main portion 9101, the delivery arm 9103, or both the main portion 9101 and the delivery arm 9103 of the implantable medical system 9000. In further under these circumstances, directionality of dispensing at the main portion 9101, the delivery arm 9103, were both the main portion 9101 and delivery arm 9103 of the implantable medical system 9000 can be the same or can be different depending on the example. The example shown and discussed in these figures are just some of many examples disclosed herein. One skilled in the art will appreciate this fact and recognize that, in reading this disclosure as a whole, certain variations and modifications of these examples are logical extensions of the examples discussed below.

Beginning with FIGS. 12A and 12B, a single direction dispensing is shown (generally into the page in FIG. 12A and downward in FIG. 12B). Distribution of dispensing is shown in a sprayed pattern where the directionality of the arrows generally deviates from one another but in the same general direction. In contrast, FIGS. 12C and 12D show a similar dispensing operation (generally into the page in FIG. 12B and downward dispensing in FIG. 12D) but in a more focused manner where the directionality of the arrows generally do not deviate from one another and are in the same direction. In FIGS. 12A through 12D, dispensing preventions are shown in a variety of other directions and locations except where dispensing is allowed.

Turning to FIGS. 12E and 12F, a multidirectional dispensing operation is shown (e.g., generally into and out of the page in FIG. 12E and upward and downward in FIG. 12F). Distribution of dispensing is shown in a focused manner (similar to FIGS. 12C and 12D) in multiple directions. As is indicated by the number of arrows, dispensing out of the page and FIG. 12E and upward in FIG. 12F is less than dispensing into the page in FIG. 12E and downward in FIG. 12F. Although shown in the same focused manner of dispensing in multiple directions, it should be appreciated that some examples include dispensing in the sprayed manner in one direction and a focused manner in another direction. It should also be appreciated that dispensing may be the same amount in each direction of dispensing or may be different amounts in each direction of dispensing. As discussed above, an amount of dispensing can be proportional to permeability at the dispensing portion of the implantable medical system 9000. FIGS. 12G and 12H show a similar dispensing operation (generally into and out of the page in FIG. 12G and upward and downward dispensing in FIG. 12H) but in a more sprayed manner (similar to FIGS. 12A and 12B) in multiple directions. In FIGS. 12E through 12G, dispensing preventions are shown in a variety of other directions and locations except where dispensing is allowed.

As noted above, FIGS. 12I-12P shows a Configuration B of the reservoir 9100 where the reservoir 9100 includes a main portion 9101 and a delivery arm 9103. Dispensing with respect to the main portion 9101 in these examples can be similar to those discussed above with reference to FIGS. 12A-12H. The main portion 9101 of the implantable medical system 9000 can be similar to those discussed with respect to FIGS. 12A-12H. The examples shown in FIGS. 12I-12P can, in addition or in alternative, include dispensing from the delivery arm 9103 as is further discussed below. While specific examples are discussed below, it should be appreciated that the skilled artisan would recognize many other examples and combinations thereof in light of this disclosure.

Turning to FIGS. 121 and 12J, a multidirectional dispensing operation at the delivery arm 9103 is shown. In this regard, dispensing at the delivery arm 9103 can occur in a variety of directions (e.g., at a tip portion of the delivery arm 9103 and along the length of the delivery arm 9103). Specific to this example, no dispensing occurs at the main portion 9101 of the implantable medical system 9000. In contrast, in the example shown in FIGS. 12M and 12N both the delivery arm 9103 and the main portion 9101 are configured to dispense in multiple directions. For instance, the main portion 9101 can dispense medicament in a similar manner as discussed with respect to FIGS. 12G and 12H, and the delivery arm 9103 can dispense medicament in a similar manner as discussed with respect to FIGS. 121 and 12J.

With respect to FIGS. 12K and 12L, a guided multidirectional dispensing operation at the delivery arm 9103 is shown. In this regard, dispensing at the delivery arm 9103 can occur in a variety of directions at only a tip portion of the delivery arm 9103. Specific to this example, no dispensing occurs at the main portion 9101 of the implantable medical system 9000 or along the length of the delivery arm 9103 separate from the tip portion. In contrast, in the example shown in FIGS. 12O and 12P the delivery arm 9103 is configured for guided multidirectional dispensing, and the main portion 9101 is configured for a focused dispensing. For instance, the main portion 9101 can dispense medicament in a similar manner as discussed with respect to FIGS. 12C and 12D, and the delivery arm 9103 can dispense medicament in a similar manner as discussed with respect to FIGS. 121 and 12J.

Arrangement of the implantable medical system 9000 at implantation locations or sites can vary depending on the disease to be treated. In examples, the implantable medical system 9000 can be arranged at the anterior location of the eye, the posterior location of the eye, or both the anterior and posterior locations of the eye. For instance, the implantable medical system 9000 can be arranged in the anterior chamber of the eye, the posterior chamber (of the anterior location) of the eye, or both the anterior and posterior locations of the eye. When arranged at the anterior location of the eye, the implantable medical system 9000 in some examples can have a portion thereof arranged in the anterior chamber. When arranged at the posterior location of the eye, the implantable medical system 9000 in some examples can have a portion thereof arranged for intravitreal administration of the medicament. In examples, the portion of the reservoir 9100 arranged in the anterior chamber or for intravitreal administration of the medicament can be the delivery arm 9103 of the reservoir 9100.

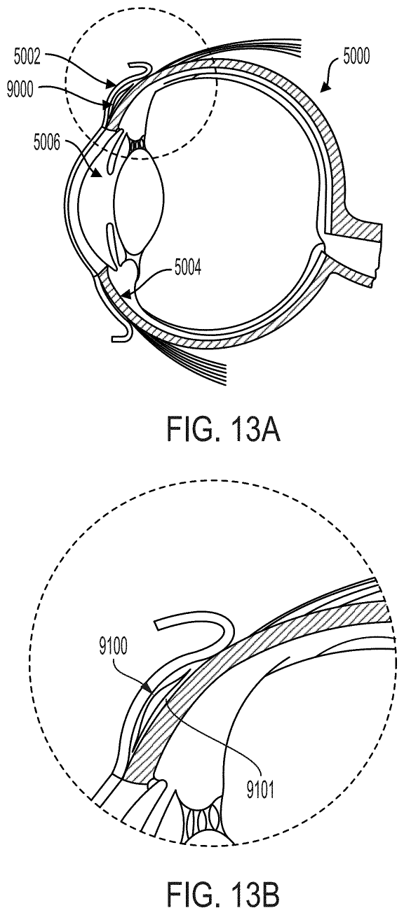

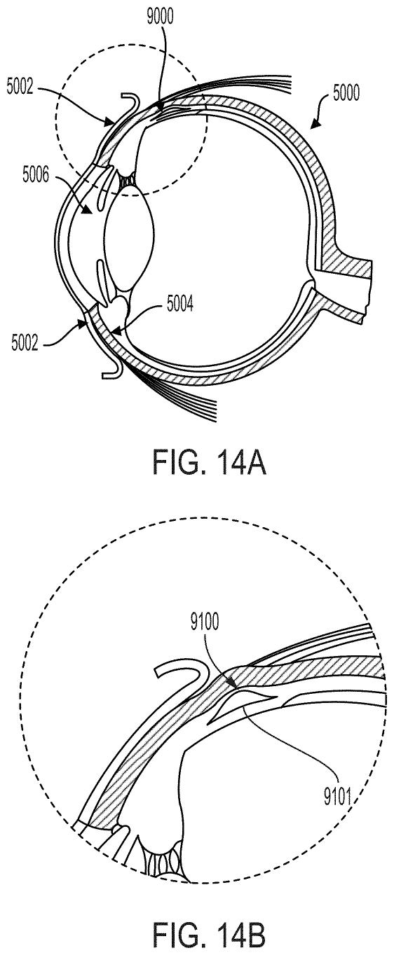

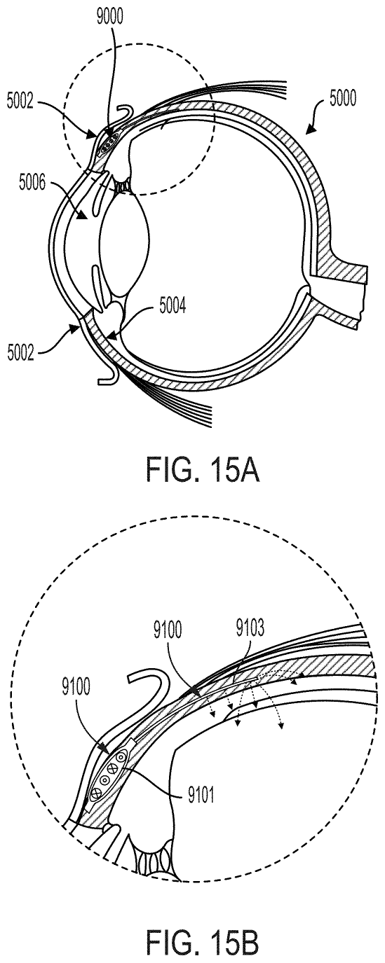

Various implantation locations or sites and configurations of the reservoir 9100 can be seen in FIGS. 13A, 13B, 14A, 14B, 15A, 15B, 16A, 16B, 17A, and 17B. As noted above, the implantable medical system 9000 can have a refillable reservoir 9100 and a delivery arm 9103 in examples. In other examples, the reservoir 9100 can have a refillable reservoir 9100 without a delivery arm 9103. Of course, in examples, the reservoir 9100 may not be refillable. In particular, FIGS. 13A, 13B, 14A, and 14B pertain to reservoirs without a delivery arm 9103, and FIGS. 15A, 15B, 16A, 16B, 17A, and 17B pertain to reservoirs with a delivery arm 9103. As is discussed elsewhere herein, the implantable medical system 9000 can be configured such that medicament is dispensed from the reservoir 9100 toward the eye (e.g., through main portion 9101, the delivery arm 9103, or both the main portion 9101 and delivery arm 9103) and optionally away from the eye 5000.

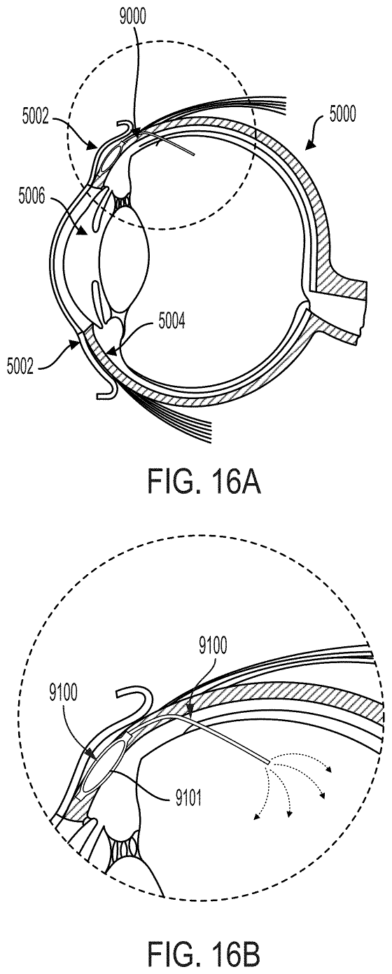

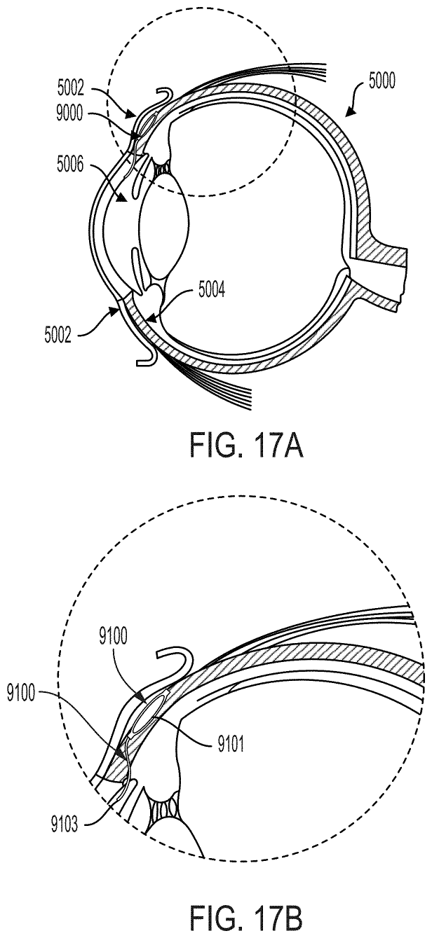

More specifically, FIGS. 13A and 13B show a medicament delivery system implanted at an anterior reservoir 9100 location (e.g., beneath the conjunctiva 5002 between the limbus and pars plana). As discussed below, such implantations can be useful for glaucoma, dry eye, presbyopia, or keratitis. FIGS. 14A and 14B show a medicament delivery system implanted at a posterior reservoir 9100 location (e.g., beyond pars plana and suprachorial). As discussed below, such implantations can be useful for macular degeneration, uveitis, retinitis, macular edema, CRVO, and BRVO. FIGS. 15A and 15B show a medicament delivery system implanted at an anterior-posterior reservoir 9100 location (e.g., reservoir 9100 between limbus and pars plana with delivery arm 9103 beyond pars plana and suprachoroidal). As discussed below, such implantations can be useful for macular degeneration, uveitis, retinitis, macular edema, CRVO, and BRVO. FIGS. 16A and 16B show a medicament delivery system implanted at an anterior-intravitreal reservoir 9100 location with a refillable reservoir 9100 positioned near the limbus and the delivery arm 9103 positioned in the vitreous chamber of the eye 5000. As discussed below, such implantations can be useful for macular edema, uveitis, retinitis, or macular degeneration. FIGS. 17A and 17B show a medicament delivery system implanted at an anterior-anterior chamber 5006 reservoir 9100 location with a refillable reservoir 9100 positioned near the limbus and the delivery arm 9103 positioned in the anterior chamber 5006. As discussed below, such implantations can be useful for glaucoma, keratitis, and presbyopia.

FIG. 18 shows a microscopic view of a microporous material according to some embodiments that presents microscopic imagery of a material structure represented illustratively in portions of FIGS. 10A and 10C. For example, the microporous material of FIG. 18 may be referred to throughout with reference to a medical implant system. As can be appreciated by a person of skill in the art and with reference to FIG. 18, the microporous aspects and parameters of the microporous material can be defined in a variety of ways. In an application of a microporous material in an ocular device, such as the medicament delivery system described herein, configured for in situ placement in the tissue of the eye to facilitate the delivery of a medicament to the eye for treatment of a disease, the microporous properties of such a microporous material can be generally characterized by a volumetric porosity value that can be defined as a ratio of a volume of the air or fluid defined by and contained within the microporous material as compared to an overall volume (or total volume) of the microporous material.

In another definition, a volumetric porosity can be defined as a percentage of the microporous material volume that is occupied by non-structural or transient elements such as air or other fluids. For example, a microporous material with an overall volume of 100 mm³ and with 30 mm³ of that volume comprising chambers holding air or a fluid would have a volumetric porosity value of 0.3 because 30% of the volume of the microporous material is empty or transient space that is filled with air or other fluids.

As can be appreciated, two microporous materials can have the same volumetric porosity but differ in the pore sizes presented to the incoming or exiting air or fluid. For example, a first material can a have a small number of large pores distributed over a fixed overall volume and a second material can have a relatively large number of relatively smaller pores distributed over the same fixed volume, and both microporous materials could have the same volumetric porosity if the air/fluid volume of the two materials are the same.

As can be further appreciated, the properties of the microporous materials used in an ocular drainage device can also be defined by the size of the passages passing through the microporous material or similarly defined as a pore size measured where a passage terminates at a surface of the microporous material or measured along a length of a passage within the material. Microporous materials with small pores or passages can impede flow through the material and comparatively large pores or passages can provide an increased pass through of the air or fluid into, out of, or within the microporous material.

As can be still further appreciated, the properties of the microporous material can also be defined by a tortuosity of the passages entering into and passing through the material, with relatively small or large passages presenting impeded fluid pathways due the frequency of turns in the passages or by the placement of obstructions in the fluid pathways. The air/fluid passthrough rates of a microporous material can be managed by controlling or defining any of the above-described characteristics of the material to provide a suitable material for use to facilitate delivery of a medicament to the eye for the treatment of a disease.

For simplicity, the aforementioned characteristics and variables of the microporous material used in the various embodiments and examples described herein can be presented simply as a porosity which can be based on a volumetric porosity, a pore or passage size, or a tortuosity metric. Again, with reference to FIG. 18, internal portions of the microporous material can have varying porosities (or volumetric porosities, or pore sizes, or tortuosities). The internal portions can extend between an inner surface 9508 and an outer surface 9510.

At any of these portions of a body portion 9502, the porosity can comparatively range in degree from small pore size (SP), medium-small pore size (MSP), medium pore size (MP), medium-large pore size (MLP), and large pore size (LP). Assuming, for discussion purposes here, that delivery travels along a relatively straight path through a microporous material so as to sequentially engage porosities of the inner surface 9508, a uniform internal portion, and the outer surface 9510, the combined flow resistance can be represented by likewise concatenating their respective porosities. For instance, the inner surface 9508 typically has a low porosity throughout (e.g., to resist tissue ingrowth into the reservoir 9504), and portions of the interior portions and the outer surface 9510 can have any of the aforementioned degrees of porosity. Under these circumstances when the internal portion has a medium porosity and, for example, the internal portions have a medium porosity and the outer surface 9510 has a high porosity, the medicament delivery through the microporous material from the reservoir 9504 to tissue surrounding the device can be represented as SP-MP-LP. More examples are discussed here below.

Various delivery paths can be present within the microporous material. Relatively linear flow paths may comprise regions SP1-SP4-SP5, for example or SP3-MLP1-MP1-MSP1. Although some flow paths may be relatively straight, there are also flow paths that are nonlinear. For instance, under certain conditions, at least some flow may proceed to flow through areas of increasingly less resistance such as SP1-LP1-LP2 or SP3-MLP1-LP1-LP2. As will be appreciated, the microstructure of the microporous materials may undergo modification processes to obtain certain types of flow through the microstructure. For instance, the microstructure may have relatively uniform layers across layered within the microstructure, or as shown here, have variable portions throughout the thickness of the microporous material.

In some examples, the body portion 9502 defines a wall portion thickness extending between the inner surface 9508 and the outer surface 9510. The wall portion thickness can define an internal region of the body portion 9502 having a transition porosity that is between a porosity of the low porosity surface (e.g., having smaller pore sizes) of the inner surface 9508 and a porosity of the high porosity surface (e.g., having larger pore sizes) of the outer surface 9510. In addition, or in alternative, the internal region can have an internal region porosity that is equal to porosities of the low porosity surfaces of the inner surface 9508 and the outer surface 9510. In addition, or in alternative, the internal region can have an internal region porosity that is equal to a porosity of the low porosity surface of the inner surface 9508. In addition, or in alternative, the internal region can have an internal region porosity that is equal to a porosity of the high porosity surface of the outer surface 9510.

With reference still to the microporous material shown in FIG. 18, the fluid pathways may also be impacted by the concentration gradient between a fluid such as water and the medicament that is within the reservoir 9100. More specifically, during the process of medicament delivery, the medicament or drug is first contained within the reservoir 9100. Fluid may then be delivered through the microporous material and into the reservoir 9100, causing the medicament to leach out of the microporous material and to the targeted delivery site. While described herein as having layers or stratum, the microporous material may lack separate and distinct layers but instead comprise different areas of varying porosity for the fluid and drug to travel though, as previously described. The medicament delivery system 9000, and more particularly the microporous material of the medicament delivery system 9000, may also be optimized for targeted delivery. In other words, the areas at which the microporous material is incorporated may be chosen in order to allow for drug delivery only in the target location around the system 9000.

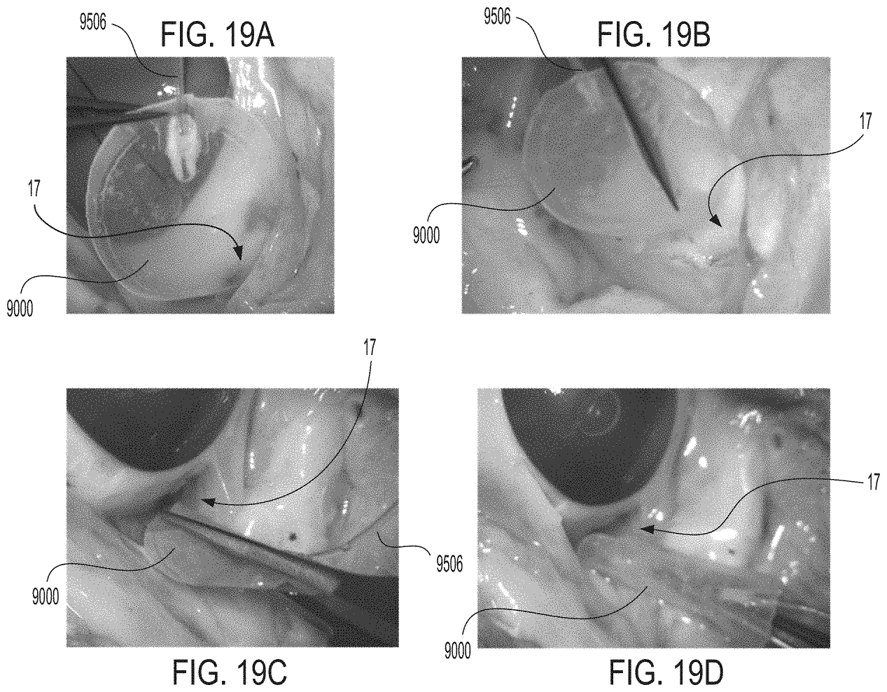

FIGS. 19A through 19D show different methods of implanting an exemplary embodiment of an implantable medical system described in the other embodiments as disclosed herein. While described with reference to the implantable medical system 9000 of FIGS. 17A-17B, the methods described herein may also be used with the system 1000 of FIGS. 1A-1B. Further, while FIGS. 19A-19D illustrate the implantation of the device into one exemplary location within the eye, the implantation process may be applied at various locations within the eye as desired and described as necessary for the above-described embodiments.

In FIG. 19A, the implantable medical system 9000 is laid flat and inserted into the cut or incision 17 into the tissue of the eye by holding the proximal end (or anterior rim, near an intake conduit 9506) using any suitable tool such as non-toothed forceps and advancing the implantable medical system 9000 into the subconjunctival space formed by the incision 17 while the pocket is held open. To prevent the device 9000 from folding or bending onto itself, the support member(s) as disclosed herein may be implemented.

In FIG. 19B, the implantable medical system 9000 is held using the forceps such that the distal end (or posterior rim) of the device 9000 is grasped to be substantially parallel with the conduit 9506. This method facilitates delivery of the device 9000 at the desired depth with little undesired longitudinal folding or bending, that is, folding or bending along the longitudinal axis defined by the forceps. In some examples, the implantable medical system 9000 may experience folding or bending along the transverse axis during the procedure, but such folding or bending is less detrimental than the longitudinal folding or bending and can be corrected by “smoothening” such folds or bends using the forceps or another tool after delivery, for example.

In FIG. 19C, the implantable medical system 9000 is folded or bent axially (that is, the fold or bend takes place either at the longitudinal axis or along a line parallel to the axis) and the distal end or posterior rim of the device 9000 is grasped using the forceps such that the forceps are substantially parallel with the conduit 9506. The device 9000 is pushed forward into the subconjunctival space inside the pocket formed by the incision 17. This method also facilitates delivery of the device 9000 at the desired depth with little undesired axial folding or bending.

In FIG. 19D, the implantable medical system 9000 is grasped on one lateral edge with the forceps approximately parallel with the conduit 9506. The device 9000 is then rolled or wrapped around the body of the forceps and subsequently pushed into the subconjunctival space. After the device 9000 is inserted, it is unrolled or unwrapped in situ within the subconjunctival space. This method also facilitates delivery of the device 9000 at the desired depth with sufficient axial stiffness and little undesired axial folding or bending.

### Glaucoma

In example implementations, the medicament delivery system can be useful for treating glaucoma. As discussed elsewhere herein, glaucoma is a progressive vision loss associated with high intraocular eye pressure. For treatment of glaucoma, a medicament delivery system can be subconjunctivally implanted (e.g., at or around a location between the limbus to the pars plana of the eye 5000 (e.g., see FIG. 13)). Amongst other advantages, for example the reduced abrasion of tissue and the refillable element of the medicament delivery system, the use of the medicament delivery system described herein for treatment of glaucoma may overcome a common issue of patient compliance when using previously established treatment methods. For example, the use of an implantable medical delivery system reduces the chance that a patient may forget to administer a daily drug, for example, through the use of eye drops.

In examples, the medicament delivery system can be any of Configurations A-D. For example, the medicament delivery system can be the implantable medical system 9000 as described previously throughout with reference to FIGS. 10A-12P and described again herein. These implantable medical systems 9000 can be similar to others disclosed elsewhere herein, including the medicament delivery systems 1000, 2000, 3000. In this regard, an implantable medical system 9000 can be implanted and arranged at one or more implantation locations or sites to deliver medicament for treatment of glaucoma.

As previously described, FIGS. 10 and 10A-10D show several examples of an implantable medical system 9000 that is useful for employing these aspects of the present disclosure. In particular, FIG. 10 is an isometric view of the implantable medical system 9000 having the reservoir 9100 with the main portion 9101 and the delivery arm 9103. FIG. 10A shows the inflated reservoir 9100 with FIGS. 10A-1 and 10A-2 therein showing a microscopic view of a first and second microporous material 9202 respectively in the implantable medical system 9000. FIG. 10B shows the reservoir 9100 in FIG. 10A being refilled via insertion of the syringe 9110 (although other suitable means for refilling the reservoir 9100 in situ may be used). In particular, FIG. 10C shows the inflated reservoir 9100 with the delivery arm 9103. FIGS. 10C-1 and 10C-2 therein showing a microscopic view of a first and second microporous material 9202 respectively in the implantable medical system 9000. FIG. 10D shows the reservoir 9100 in FIG. 10C being refilled via insertion of the syringe 9110 (although other suitable means for refilling the reservoir 9100 in situ may be used). In examples, the main portion 9101 and the delivery arm 9103 can comprise the same or similar materials. Under these circumstances, the delivery arm 9103 can be made integral with or attachable to the main portion 9101 such that the delivery arm 9103 and the main portion 9101 are in fluid communication with each other. The example microporous material is shown as a schematic in FIGS. 10A-1 and 10A-2 for clarity and illustration purposes. The illustrative imagery provided in FIGS. 10A-1, 10A-2, 10C-1, and 10C-2 are representative of the microscopic imagery presented in FIG. 18. Further discussion of microporous materials that are appropriate for employing principles of the present disclosure are shown and described in U.S. Pat. Pub. US2018/0263817, the entire contents of which are hereby incorporated by reference.

The medicant delivery device shown here in FIGS. 10A-10D can be similar to the implantable delivery devices 9000 discussed above. For instance, this device can be used to dispense medicament as discussed above. The implantable medical system 9000 can include the material structure presented in Details 10A-1 and 10C-1 and shown in FIG. 18. The implantable medical system 9000 can also include the material structure, selective permeability, and porous materials shown and described in U.S. Pat. No. 10,849,731 to Cully and as shown and described in U.S. Pat. Publ. No. 2018/0126134 to Cully, both of which are incorporated by reference in their entireties, with particular reference to FIGS. 3-10 and the accompanying descriptions provided in each of those incorporated references. The implantable medical system 9000 can further include the first microporous material 9201 that is bonded to the second microporous material 9202. The first microporous material 9201 can have the first microporous layer 9203 that includes the plurality of pores sized to permit tissue ingrowth. The first microporous material 9201 can have the second microporous layer 9205 that includes the plurality of pores sized to permit tissue ingrowth. The second microporous material 9202 can have the third microporous layer 9205 that includes the plurality of pores sized to resist tissue ingrowth. The second microporous material 9202 can have the fourth microporous layer 9207 that includes the plurality of pores sized to permit tissue ingrowth. The second microporous layer 9205 can be bonded to the third microporous layer 9205 to thereby form the reservoir 9100 for receiving the medicament. The first and second microporous materials 9201 and 9202 can be configured to meter the rate at which the medicament is dispensed from the reservoir 9100 when the delivery device is implanted.

According to principles of the present disclosure, the implantable medical system 9000 can be used to treat glaucoma. In general, methods of treating diseases using an implantable medical system 9000 configured to meter a rate at which the medicament is dispensed from the reservoir 9100 when the delivery device is implanted are disclosed. The method can include selecting an implantable medical system 9000 that is similar to the implantable medical system 9000 discussed above. For instance, the first microporous material 9201 can have a first microporous layer 9203 that includes a plurality of pores sized to resist tissue ingrowth. The first microporous material 9201 can have a second microporous layer 9205 that includes a plurality of pores sized to permit tissue ingrowth. The second microporous material 9202 can have a third microporous layer 9205 that includes a plurality of pores sized to resist tissue ingrowth. The second microporous material 9202 can have a fourth microporous layer 9207 that includes a plurality of pores sized to permit tissue ingrowth. The second microporous layer 9205 can be bonded to the third microporous layer 9205 to thereby form a reservoir 9100 for receiving the medicament. The first and second microporous materials 9201 and 9202 can be configured to meter a rate at which the glaucoma medicament is dispensed from the reservoir 9100 when the delivery device is implanted.

Administration of the medicament can include several steps. For instance, the method can include filling the reservoir 9100 with a medicament for treating glaucoma. In certain instances, portions or all of the first and second microporous material 9202 can be elastomeric such that they reseal after insertion of a syringe 9110 or other similar devices. The method can include implanting the implantable medical system 9000 at an implantation location. The method can include allowing the medicament to dispense from the reservoir 9100 to one or more treatment sites.

FIGS. 11A-11D show various features of the reservoir 9100 in examples of medicament treatment devices. In particular, FIG. 11A shows the first configuration (Configuration “A”) of the reservoir 9100 where the reservoir 9100 includes the main portion 9101 only. FIG. 11B shows the second configuration (Configuration “B”) of the reservoir 9100 where the reservoir 9100 includes the main portion 9101 and the delivery arm 9103. FIG. 11C shows the third configuration (Configuration “C”) of the reservoir 9100 that is similar to that of FIG. 11A and further includes the plurality of chambers 9300 and the plurality of fill ports 9310 with which to fill the corresponding chamber with medicament. FIG. 11D shows the fourth configuration (Configuration “D”) of the reservoir 9100 that is similar to that of FIG. 11B and further includes the plurality of chambers 9300 and the plurality of fill ports 9310 with which to fill the corresponding chamber with medicament. In examples, fill ports 9310 can be an injection site at the reservoir 9100 or a feature of the implantable medical system 9000 that facilitates filling the reservoir 9100 with medicament. As further discussed below, the plurality of chambers 9300 (e.g., in FIGS. 11C and 11D) can be in fluid communication or fluidically isolated from one another. In examples, as further discussed elsewhere herein, the reservoir 9100 can dispense medicament in one direction (e.g., into the page) or in multiple directions (e.g., into the page, out of the page, in-plane with the page, or any combination thereof).

A number of factors can influence physical characteristics of medicament treatment devices. Depending on at least one of the implantation locations and the disease to be treated, one or both of a geometry and functionality of the reservoir 9100 of the medicament delivery system 1000 can vary in examples. For instance, dimensions the reservoir 9100 can be made larger or smaller when compared to other configurations of the medicament delivery system 1000. These variations can depend on, among other things, the amount of medicament to be delivered for treatment, the manner in which the medicament is to be delivered, and the resistance to delivery of medicament presented by an implantation location. As discussed here, in examples, the reservoir 9100 can have a generally uniform shape (e.g., resembling an ellipse, a circle, a polygon, etc.). In other examples, the reservoir 9100 can have an irregular shape (e.g., a generally uniform shape with one or more extrusions or protrusions therefrom, an eccentric shape, etc.). Some example shapes of the reservoir 9100 are discussed below with respect to certain example implementations of the medicament delivery system 1000. In examples, when the reservoir 9100 is inflated, the reservoir 9100 can bulge about along a length of the reservoir 9100. In some instances, bulging may cause the reservoir 9100 to balloon or to inflate to a generally polyhedron shape.

In certain examples, the physical characteristics of the reservoir 9100 can facilitate functionality of the medicament delivery system 1000. For instance, the reservoir 9100 can be formed to deliver medicament to a pinpoint location relative to the reservoir 9100, generally disperse medicament over a treatment site at one or more sides of the medicament delivery system 1000, and the like. In this regard, it can be said that the implantable medical system 9000 can have one-directional dispensing (e.g., at one side of the implantable medical system 9000) or multidirectional dispensing (e.g., at multiple sides of the implantable medical system 9000). In examples, one or more locations at the reservoir 9100 can function as the fill port 9310 with which to fill the reservoir 9100 with medicament. In this regard, the implantable medical system 9000 can be refillable with medicament as further discussed above. For example, the main portion 9101 of the reservoir 9100 can function as a refillable chamber, and the delivery arm 9103 can function as a conduit with which to deliver fluid from the refillable chamber. After implantation, medicament can be flushed or drained from the fill port 9310 at the reservoir 9100 to a treatment location. In examples, the reservoir 9100 can include the plurality of chambers 9300 therein with which to store medicament to be delivered. Under these circumstances, one chamber in the plurality of chambers 9300 can include a first medicament, and another chamber in the plurality of chambers 9300 can include a second medicament that is the same or different from the first medicament. Chambers 9300 in the plurality of chambers 9300 can be in fluid communication with one another or isolated from one another depending on the implementation.

Formed at the periphery of the reservoir 9100 can be the extended delivery arm 9103 through which medicament from the reservoir 9100 can be administered. In this regard, the reservoir 9100 can include the reservoir main portion 9101 and the extended delivery arm 9103 extending from the reservoir main portion 9101. For instance, the extended delivery arm 9103 can include a port through which medicament is exclusively allowed to exit the reservoir 9100. In other instances, medicament can be allowed exit the reservoir 9100 at any portion (e.g., not exclusively at the port) of the extended delivery arm 9103. In addition, or in alternative, medicament can be allowed to exit the reservoir 9100 both at the reservoir main portion 9101 and the extended delivery arm 9103.

FIGS. 12A-12P show various dispensing configurations of the reservoir 9100. In particular, FIGS. 12A-12H show the implantable medical system 9000 dispensing when in Configuration A of the reservoir 9100 where the reservoir 9100 includes the main portion 9101 only. FIGS. 12I-12P shows a Configuration B of the reservoir 9100 where the reservoir 9100 includes the main portion 9101 and the delivery arm 9103. Although for conciseness, the dispensing operations here are shown with respect to Configurations A and B, one skilled in the art will appreciate that these dispensing operations are similarly applicable to Configurations C and D. In this regard, the distinction would be that Configurations C and D include multiple chambers that may be similarly dispensed to the single chambers discussed below with respect to Configurations A and B. For illustration purposes, dispensing directions are shown as straight arrows flowing out of the reservoir 9100, and dispensing preventions are shown as curly arrows at the interior of the reservoir 9100. Dispensing can occur at only portions of the implantable medical system 9000 that are permeable, and dispensing preventions can occur at impermeable portions of the implantable medical system 9000. In addition, flow and directions into the page are shown as an encircled X, and directions out of the page are shown as encircled dots. Of course, these are merely schematic representations of a more complex phenomena but are used here for clarity of discussion.

As will be discussed below in further detail, dispensing of the medicament can vary across configurations and within configurations. For instance, as discussed above, the implantable medical system 9000 can be configured such that medicament is generally dispensed in one direction or in a plurality of directions. Under these circumstances, dispensing can occur at the main portion 9101, the delivery arm 9103, or both the main portion 9101 and the delivery arm 9103 of the implantable medical system 9000. In further under these circumstances, directionality of dispensing at the main portion 9101, the delivery arm 9103, or both the main portion 9101 and delivery arm 9103 of the implantable medical system 9000 can be the same or can be different depending on the example. The example shown and discussed in these figures are just some of many examples disclosed herein. One skilled in the art will appreciate this fact and recognize that, in reading this disclosure as a whole, certain variations and modifications of these examples are logical extensions of the examples discussed below.

With reference again to FIGS. 12A and 12B, a single direction dispensing is shown (generally into the page in FIG. 12A and downward in FIG. 12B). Distribution of dispensing is shown in a sprayed pattern where the directionality of the arrows generally deviates from one another but in the same general direction. In contrast, FIGS. 12C and 12D show a similar dispensing operation (generally into the page in FIG. 12B and downward dispensing in FIG. 12D) but in a more focused manner where the directionality of the arrows generally do not deviate from one another and are in the same direction. In FIGS. 12A through 12D, dispensing preventions are shown in a variety of other directions and locations except where dispensing is allowed.

Turning again to FIGS. 12E and 12F, a multidirectional dispensing operation is shown (e.g., generally into and out of the page in FIG. 12E and upward and downward in FIG. 12F). Distribution of dispensing is shown in a focused manner (similar to FIGS. 12C and 12D) in multiple directions. As is indicated by the number of arrows, dispensing out of the page and FIG. 12E and upward in FIG. 12F is less than dispensing into the page in FIG. 12E and downward in FIG. 12F. Although shown in the same focused manner of dispensing in multiple directions, it should be appreciated that some examples include dispensing in the sprayed manner in one direction and a focused manner in another direction. It should also be appreciated that dispensing may be the same amount in each direction of dispensing or may be different amounts in each direction of dispensing. As discussed above, an amount of dispensing can be proportional to permeability at the dispensing portion of the implantable medical system 9000. As previously described, FIGS. 12G and 12H show a similar dispensing operation (generally into and out of the page in FIG. 12G and upward and downward dispensing in FIG. 12H) but in a more sprayed manner (similar to FIGS. 12A and 12B) in multiple directions. In FIGS. 12E through 12G, dispensing preventions are shown in a variety of other directions and locations except where dispensing is allowed.

As noted above, FIGS. 12I-12P shows a Configuration B of the reservoir 9100 where the reservoir 9100 includes a main portion 9101 and a delivery arm 9103. Dispensing with respect to the main portion 9101 in these examples can be similar to those discussed above with reference to FIGS. 12A-12H. The main portion 9101 of the implantable medical system 9000 can be similar to those discussed with respect to FIGS. 12A-12H. The examples shown in FIGS. 12I-12P can, in addition or in alternative, include dispensing from the delivery arm 9103 as is further discussed below. While specific examples are discussed below, it should be appreciated that the skilled artisan would recognize many other examples and combinations thereof in light of this disclosure.

Turning to FIGS. 121 and 12J, a multidirectional dispensing operation at the delivery arm 9103 is shown. In this regard, dispensing at the delivery arm 9103 can occur in a variety of directions (e.g., at a tip portion of the delivery arm 9103 and along the length of the delivery arm 9103). Specific to this example, no dispensing occurs at the main portion 9101 of the implantable medical system 9000. In contrast, in the example shown in FIGS. 12M and 12N both the delivery arm 9103 and the main portion 9101 are configured to dispense in multiple directions. For instance, the main portion 9101 can dispense medicament in a similar manner as discussed with respect to FIGS. 12G and 12H, and the delivery arm 9103 can dispense medicament in a similar manner as discussed with respect to FIGS. 121 and 12J.

As previously discussed with reference to FIGS. 12K and 12L, a guided multidirectional dispensing operation at the delivery arm 9103 is shown. In this regard, dispensing at the delivery arm 9103 can occur in a variety of directions at only a tip portion of the delivery arm 9103. Specific to this example, no dispensing occurs at the main portion 9101 of the implantable medical system 9000 or along the length of the delivery arm 9103 separate from the tip portion. In contrast, in the example shown in FIGS. 12O and 12P the delivery arm 9103 is configured for guided multidirectional dispensing, and the main portion 9101 is configured for a focused dispensing. For instance, the main portion 9101 can dispense medicament in a similar manner as discussed with respect to FIGS. 12C and 12D, and the delivery arm 9103 can dispense medicament in a similar manner as discussed with respect to FIGS. 121 and 12J.

Any of the above configurations of the implantable medical system 100 and dispensing mechanisms may be applied for use in treating glaucoma. Under these circumstances, the medicament delivery system can be arranged at the anterior location of the eye 5000 and optionally in the anterior chamber 5006 of the eye 5000 (e.g., see FIG. 17). For instance, when the medicament delivery system is so arranged to treat glaucoma, API classes can include prostaglandins such as latanoprost, bimatoprost, and XALATAN. In certain instances, API classes can include beta blockers such as timolol or BETIMOL; alpha agonist such as brimonidine or ALPHAGEN; or cabonic anhydrase inhibitors such as dorzolamide, brinzolamide, or AZOPT.

### Macular Degeneration

The present disclosure includes devices, systems and methods suitable for treatment of retinal diseases. In particular, macular degeneration of the retina can be treated using example implementations of the implantable medical system 9000. Wet macular degeneration is a chronic eye disorder that involves abnormal blood vessel growth under the macula, which is responsible for central vision of the eye 5000. In previously established treatments for macular degeneration, a needle is inserted into the eye and breaches the blood aqueous barrier. One of the advantages of the currently presented embodiment, amongst other advantages, is the ability for this invasive technique to be eliminated and for the treatment to be delivered minimally invasively, as will be described further herein.

For treatment of macular degeneration, a medicament delivery system can be suprachoroidally implanted (e.g., posterior of the pars plana of the eye 5000). In examples, the medicament delivery system can be any of Configurations A-D. For example, the medicament delivery system can be the implantable medical system 9000 as described previously throughout with reference to FIGS. 10A-12P and described again herein. These implantable medical systems 9000 can be similar to others disclosed elsewhere herein, including the medicament delivery systems 1000, 2000, 3000. In this regard, an implantable medical system 9000 can be implanted and arranged at one or more implantation locations or sites to deliver medicament for treatment of macular degeneration.

As previously described, FIGS. 10 and 10A-10D show several examples of an implantable medical system 9000 that is useful for employing these aspects of the present disclosure. In particular, FIG. 10 is an isometric view of the implantable medical system 9000 having the reservoir 9100 with the main portion 9101 and the delivery arm 9103. FIG. 10A shows the inflated reservoir 9100 with FIGS. 10A-110A-2 therein showing a microscopic view of a first and second microporous material 9202 respectively in the implantable medical system 9000. FIG. 10B shows the reservoir 9100 in FIG. 10A being refilled via insertion of the syringe 9110 (although other suitable means for refilling the reservoir 9100 in situ may be used). In particular, FIG. 10C shows the inflated reservoir 9100 with the delivery arm 9103. FIGS. 10C-1 and 10C-2 therein showing a microscopic view of a first and second microporous material 9202 respectively in the implantable medical system 9000. FIG. 10D shows the reservoir 9100 in FIG. 10C being refilled via insertion of the syringe 9110 (although other suitable means for refilling the reservoir 9100 in situ may be used). In examples, the main portion 9101 and the delivery arm 9103 can comprise the same or similar materials. Under these circumstances, the delivery arm 9103 can be made integral with or attachable to the main portion 9101 such that the delivery arm 9103 and the main portion 9101 are in fluid communication with each other. The example microporous material is shown as a schematic in Details A and B for clarity and illustration purposes. The illustrative imagery provided in FIGS. 10A-1, 10A-2, 10C-1 and 10C-2 are representative of the microscopic imagery presented in FIG. 18. Further discussion of microporous materials that are appropriate for employing principles of the present disclosure are shown and described in U.S. Pat. Pub. US2018/0263817, the entire contents of which are hereby incorporated by reference.

The medicant delivery device shown here in FIGS. 10A-10D can be similar to the implantable delivery devices 9000 discussed above. For instance, this device can be used to dispense medicament as discussed above. The implantable medical system 9000 can include the material structure presented in FIGS. 10A-1 and 10C-1 and shown in FIG. 18. The implantable medical system 9000 can also include the material structure, selective permeability, and porous materials shown and described in U.S. Pat. No. 10,849,731 to Cully and as shown and described in U.S. Pat. Publ. No. 2018/0126134 to Cully, both of which are incorporated by reference in their entireties, with particular reference to FIGS. 3-10 and the accompanying descriptions provided in each of those incorporated references. The implantable medical system 9000 can further include the first microporous material 9201 that is bonded to the second microporous material 9202. The first microporous material 9201 can have the first microporous layer 9203 that includes the plurality of pores sized to permit tissue ingrowth. The first microporous material 9201 can have the second microporous layer 9205 that includes the plurality of pores sized to permit tissue ingrowth. The second microporous material 9202 can have the third microporous layer 9205 that includes the plurality of pores sized to resist tissue ingrowth. The second microporous material 9202 can have the fourth microporous layer 9207 that includes the plurality of pores sized to permit tissue ingrowth. The second microporous layer 9205 can be bonded to the third microporous layer 9205 to thereby form the reservoir 9100 for receiving the medicament. The first and second microporous materials 9201 and 9202 can be configured to meter the rate at which the medicament is dispensed from the reservoir 9100 when the delivery device is implanted.

According to principles of the present disclosure, the implantable medical system 9000 can be used to treat macular degeneration. In general, methods of treating diseases using an implantable medical system 9000 configured to meter a rate at which the medicament is dispensed from the reservoir 9100 when the delivery device is implanted are disclosed. The method can include selecting an implantable medical system 9000 that is similar to the implantable medical system 9000 discussed above. For instance, the first microporous material 9201 can have a first microporous layer 9203 that includes a plurality of pores sized to resist tissue ingrowth. The first microporous material 9201 can have a second microporous layer 9205 that includes a plurality of pores sized to permit tissue ingrowth. The second microporous material 9202 can have a third microporous layer 9205 that includes a plurality of pores sized to resist tissue ingrowth. The second microporous material 9202 can have a fourth microporous layer 9207 that includes a plurality of pores sized to permit tissue ingrowth. The second microporous layer 9205 can be bonded to the third microporous layer 9205 to thereby form a reservoir 9100 for receiving the medicament. The first and second microporous materials 9201 and 9202 can be configured to meter a rate at which the macular degeneration medicament is dispensed from the reservoir 9100 when the delivery device is implanted.

Administration of the medicament can include several steps. For instance, the method can include filling the reservoir 9100 with a medicament for treating macular degeneration. In certain instances, portions or all of the first and second microporous material 9202 can be elastomeric such that they reseal after insertion of a syringe 9110 or other similar devices. The method can include implanting the implantable medical system 9000 at an implantation location. The method can include allowing the medicament to dispense from the reservoir 9100 to one or more treatment sites.

FIGS. 11A-11D show various features of the reservoir 9100 in examples of medicament treatment devices. In particular, FIG. 11A shows the first configuration (Configuration “A”) of the reservoir 9100 where the reservoir 9100 includes the main portion 9101 only. FIG. 11B shows the second configuration (Configuration “B”) of the reservoir 9100 where the reservoir 9100 includes the main portion 9101 and the delivery arm 9103. FIG. 11C shows the third configuration (Configuration “C”) of the reservoir 9100 that is similar to that of FIG. 11A and further includes the plurality of chambers 9300 and the plurality of fill ports 9310 with which to fill the corresponding chamber with medicament. FIG. 11D shows the fourth configuration (Configuration “D”) of the reservoir 9100 that is similar to that of FIG. 11B and further includes the plurality of chambers 9300 and the plurality of fill ports 9310 with which to fill the corresponding chamber with medicament. In examples, fill ports 9310 can be an injection site at the reservoir 9100 or a feature of the implantable medical system 9000 that facilitates filling the reservoir 9100 with medicament. As further discussed below, the plurality of chambers 9300 (e.g., in FIGS. 11C and 11D) can be in fluid communication or fluidically isolated from one another. In examples, as further discussed elsewhere herein, the reservoir 9100 can dispense medicament in one direction (e.g., into the page) or in multiple directions (e.g., into the page, out of the page, in-plane with the page, or any combination thereof).

A number of factors can influence physical characteristics of medicament treatment devices. Depending on at least one of the implantation locations and the disease to be treated, one or both of a geometry and functionality of the reservoir 9100 of the medicament delivery system 1000 can vary in examples. For instance, dimensions the reservoir 9100 can be made larger or smaller when compared to other configurations of the medicament delivery system 1000. These variations can depend on, among other things, the amount of medicament to be delivered for treatment, the manner in which the medicament is to be delivered, and the resistance to delivery of medicament presented by an implantation location. As discussed here, in examples, the reservoir 9100 can have a generally uniform shape (e.g., resembling an ellipse, a circle, a polygon, etc.). In other examples, the reservoir 9100 can have an irregular shape (e.g., a generally uniform shape with one or more extrusions or protrusions therefrom, an eccentric shape, etc.). Some example shapes of the reservoir 9100 are discussed below with respect to certain example implementations of the medicament delivery system 1000. In examples, when the reservoir 9100 is inflated, the reservoir 9100 can bulge about along a length of the reservoir 9100. In some instances, bulging may cause the reservoir 9100 to balloon or to inflate to a generally polyhedron shape.

In certain examples, the physical characteristics of the reservoir 9100 can facilitate functionality of the medicament delivery system 1000. For instance, the reservoir 9100 can be formed to deliver medicament to a pinpoint location relative to the reservoir 9100, generally disperse medicament over a treatment site at one or more sides of the medicament delivery system 1000, and the like. In this regard, it can be said that the implantable medical system 9000 can have one-directional dispensing (e.g., at one side of the implantable medical system 9000) or multidirectional dispensing (e.g., at multiple sides of the implantable medical system 9000). In examples, one or more locations at the reservoir 9100 can function as the fill port 9310 with which to fill the reservoir 9100 with medicament. In this regard, the implantable medical system 9000 can be refillable with medicament as further discussed above. For example, the main portion 9101 of the reservoir 9100 can function as a refillable chamber, and the delivery arm 9103 can function as a conduit with which to deliver fluid from the refillable chamber. After implantation, medicament can be flushed or drained from the fill port 9310 at the reservoir 9100 to a treatment location. In examples, the reservoir 9100 can include the plurality of chambers 9300 therein with which to store medicament to be delivered. Under these circumstances, one chamber in the plurality of chambers 9300 can include a first medicament, and another chamber in the plurality of chambers 9300 can include a second medicament that is the same or different from the first medicament. Chambers 9300 in the plurality of chambers 9300 can be in fluid communication with one another or isolated from one another depending on the implementation.

Formed at the periphery of the reservoir 9100 can be the extended delivery arm 9103 through which medicament from the reservoir 9100 can be administered. In this regard, the reservoir 9100 can include the reservoir main portion 9101 and the extended delivery arm 9103 extending from the reservoir main portion 9101. For instance, the extended delivery arm 9103 can include a port through which medicament is exclusively allowed to exit the reservoir 9100. In other instances, medicament can be allowed exit the reservoir 9100 at any portion (e.g., not exclusively at the port) of the extended delivery arm 9103. In addition, or in alternative, medicament can be allowed to exit the reservoir 9100 both at the reservoir main portion 9101 and the extended delivery arm 9103.

FIGS. 12A-12P show various dispensing configurations of the reservoir 9100. In particular, FIGS. 12A-12H show the implantable medical system 9000 dispensing when in Configuration A of the reservoir 9100 where the reservoir 9100 includes the main portion 9101 only. FIGS. 12I-12P shows a Configuration B of the reservoir 9100 where the reservoir 9100 includes the main portion 9101 and the delivery arm 9103. Although for conciseness, the dispensing operations here are shown with respect to Configurations A and B, one skilled in the art will appreciate that these dispensing operations are similarly applicable to Configurations C and D. In this regard, the distinction would be that Configurations C and D include multiple chambers that may be similarly dispensed to the single chambers discussed below with respect to Configurations A and B. For illustration purposes, dispensing directions are shown as straight arrows flowing out of the reservoir 9100, and dispensing preventions are shown as curly arrows at the interior of the reservoir 9100. Dispensing can occur at only portions of the implantable medical system 9000 that are permeable, and dispensing preventions can occur at impermeable portions of the implantable medical system 9000. In addition, flow and directions into the page are shown as an encircled X, and directions out of the page are shown as encircled dots. Of course, these are merely schematic representations of a more complex phenomena but are used here for clarity of discussion.

As will be discussed below in further detail, dispensing of the medicament can vary across configurations and within configurations. For instance, as discussed above, the implantable medical system 9000 can be configured such that medicament is generally dispensed in one direction or in a plurality of directions. Under these circumstances, dispensing can occur at the main portion 9101, the delivery arm 9103, or both the main portion 9101 and the delivery arm 9103 of the implantable medical system 9000. In further under these circumstances, directionality of dispensing at the main portion 9101, the delivery arm 9103, or both the main portion 9101 and delivery arm 9103 of the implantable medical system 9000 can be the same or can be different depending on the example. The example shown and discussed in these figures are just some of many examples disclosed herein. One skilled in the art will appreciate this fact and recognize that, in reading this disclosure as a whole, certain variations and modifications of these examples are logical extensions of the examples discussed below.

With reference again to FIGS. 12A and 12B, a single direction dispensing is shown (generally into the page in FIG. 12A and downward in FIG. 12B). Distribution of dispensing is shown in a sprayed pattern where the directionality of the arrows generally deviates from one another but in the same general direction. In contrast, FIGS. 12C and 12D show a similar dispensing operation (generally into the page in FIG. 12B and downward dispensing in FIG. 12D) but in a more focused manner where the directionality of the arrows generally do not deviate from one another and are in the same direction. In FIGS. 12A through 12D, dispensing preventions are shown in a variety of other directions and locations except where dispensing is allowed.

Turning again to FIGS. 12E and 12F, a multidirectional dispensing operation is shown (e.g., generally into and out of the page in FIG. 12E and upward and downward in FIG. 12F). Distribution of dispensing is shown in a focused manner (similar to FIGS. 12C and 12D) in multiple directions. As is indicated by the number of arrows, dispensing out of the page and FIG. 12E and upward in FIG. 12F is less than dispensing into the page in FIG. 12E and downward in FIG. 12F. Although shown in the same focused manner of dispensing in multiple directions, it should be appreciated that some examples include dispensing in the sprayed manner in one direction and a focused manner in another direction. It should also be appreciated that dispensing may be the same amount in each direction of dispensing or may be different amounts in each direction of dispensing. As discussed above, an amount of dispensing can be proportional to permeability at the dispensing portion of the implantable medical system 9000. As previously described, FIGS. 12G and 12H show a similar dispensing operation (generally into and out of the page in FIG. 12G and upward and downward dispensing in FIG. 12H) but in a more sprayed manner (similar to FIGS. 12A and 12B) in multiple directions. In FIGS. 12E through 12G, dispensing preventions are shown in a variety of other directions and locations except where dispensing is allowed.

As noted above, FIGS. 12I-12P shows a Configuration B of the reservoir 9100 where the reservoir 9100 includes a main portion 9101 and a delivery arm 9103. Dispensing with respect to the main portion 9101 in these examples can be similar to those discussed above with reference to FIGS. 12A-12H. The main portion 9101 of the implantable medical system 9000 can be similar to those discussed with respect to FIGS. 12A-12H. The examples shown in FIGS. 12I-12P can, in addition or in alternative, include dispensing from the delivery arm 9103 as is further discussed below. While specific examples are discussed below, it should be appreciated that the skilled artisan would recognize many other examples and combinations thereof in light of this disclosure.

Turning to FIGS. 121 and 12J, a multidirectional dispensing operation at the delivery arm 9103 is shown. In this regard, dispensing at the delivery arm 9103 can occur in a variety of directions (e.g., at a tip portion of the delivery arm 9103 and along the length of the delivery arm 9103). Specific to this example, no dispensing occurs at the main portion 9101 of the implantable medical system 9000. In contrast, in the example shown in FIGS. 12M and 12N both the delivery arm 9103 and the main portion 9101 are configured to dispense in multiple directions. For instance, the main portion 9101 can dispense medicament in a similar manner as discussed with respect to FIGS. 12G and 12H, and the delivery arm 9103 can dispense medicament in a similar manner as discussed with respect to FIGS. 121 and 12J.

As previously discussed with reference to FIGS. 12K and 12L, a guided multidirectional dispensing operation at the delivery arm 9103 is shown. In this regard, dispensing at the delivery arm 9103 can occur in a variety of directions at only a tip portion of the delivery arm 9103. Specific to this example, no dispensing occurs at the main portion 9101 of the implantable medical system 9000 or along the length of the delivery arm 9103 separate from the tip portion. In contrast, in the example shown in FIGS. 12O and 12P the delivery arm 9103 is configured for guided multidirectional dispensing, and the main portion 9101 is configured for a focused dispensing. For instance, the main portion 9101 can dispense medicament in a similar manner as discussed with respect to FIGS. 12C and 12D, and the delivery arm 9103 can dispense medicament in a similar manner as discussed with respect to FIGS. 121 and 12J.

Any of the above configurations of the implantable medical system 100 and dispensing mechanisms may be applied for use in treating macular degeneration. Under these circumstances, the medicament delivery system (e.g., in Configurations A and C) can be arranged at the posterior location of the eye 5000 (e.g., see FIG. 14) and optionally (e.g., in Configurations B and D) at the anterior location of the eye 5000 (e.g., see FIG. 15). In some such circumstances, the medicament delivery system (e.g., in Configurations B and D) is positioned at the anterior location of the eye 5000 and arranged for intravitreal administration. API classes can include monoclonal antibodies such as bevacizumab, ranibizumab, afibercept, AVASTIN, LUCENTIS, or EYLEA.

### Macular Edema

Other retinal diseases such as macular edema of the retina can be treated using example implementations of the implantable medical system 9000. Macular edema is a chronic eye disorder that involves distorted vision by swelling of the macula, which is responsible for central vision of the eye 5000. For treatment of macular edema, a medicament delivery system can be suprachoroidally implanted (e.g., posterior of the pars plana of the eye 5000). In examples, the medicament delivery system can be any of Configurations A-D. For example, the medicament delivery system can be the implantable medical system 9000 as described previously throughout with reference to FIGS. 10A-12P and described again herein. These implantable medical systems 9000 can be similar to others disclosed elsewhere herein, including the medicament delivery systems 1000, 2000, 3000. In this regard, an implantable medical system 9000 can be implanted and arranged at one or more implantation locations or sites to deliver medicament for treatment of macular edema.

As previously described, FIGS. 10 and 10A-10D show several examples of an implantable medical system 9000 that is useful for employing these aspects of the present disclosure. In particular, FIG. 10 is an isometric view of the implantable medical system 9000 having the reservoir 9100 with the main portion 9101 and the delivery arm 9103. FIG. 10A shows the inflated reservoir 9100 with FIGS. 10A-1 and FIGS. 10A-2 therein showing a microscopic view of a first and second microporous material 9202 respectively in the implantable medical system 9000. FIG. 10B shows the reservoir 9100 in FIG. 10A being refilled via insertion of the syringe 9110 (although other suitable means for refilling the reservoir 9100 in situ may be used). In particular, FIG. 10C shows the inflated reservoir 9100 with the delivery arm 9103. Details C and D therein showing a microscopic view of a first and second microporous material 9202 respectively in the implantable medical system 9000. FIG. 10D shows the reservoir 9100 in FIG. 10C being refilled via insertion of the syringe 9110 (although other suitable means for refilling the reservoir 9100 in situ may be used). In examples, the main portion 9101 and the delivery arm 9103 can comprise the same or similar materials. Under these circumstances, the delivery arm 9103 can be made integral with or attachable to the main portion 9101 such that the delivery arm 9103 and the main portion 9101 are in fluid communication with each other. The example microporous material is shown as a schematic in FIGS. 10A-1 and 10A-2 for clarity and illustration purposes. The illustrative imagery provided in FIGS. 10A-1, 10A-2, 10C-1, and 10C-2 are representative of the microscopic imagery presented in FIG. 18. Further discussion of microporous materials that are appropriate for employing principles of the present disclosure are shown and described in U.S. Pat. Pub. US2018/0263817, the entire contents of which are hereby incorporated by reference.

The medicant delivery device shown here in FIGS. 10A-10D can be similar to the implantable delivery devices 9000 discussed above. For instance, this device can be used to dispense medicament as discussed above. The implantable medical system 9000 can include the material structure presented in FIGS. 10A-1 and 10C-1 and shown in FIG. 18. The implantable medical system 9000 can also include the material structure, selective permeability, and porous materials shown and described in U.S. Pat. No. 10,849,731 to Cully and as shown and described in U.S. Pat. Publ. No. 2018/0126134 to Cully, both of which are incorporated by reference in their entireties, with particular reference to FIGS. 3-10 and the accompanying descriptions provided in each of those incorporated references. The implantable medical system 9000 can further include the first microporous material 9201 that is bonded to the second microporous material 9202. The first microporous material 9201 can have the first microporous layer 9203 that includes the plurality of pores sized to permit tissue ingrowth. The first microporous material 9201 can have the second microporous layer 9205 that includes the plurality of pores sized to permit tissue ingrowth. The second microporous material 9202 can have the third microporous layer 9205 that includes the plurality of pores sized to resist tissue ingrowth. The second microporous material 9202 can have the fourth microporous layer 9207 that includes the plurality of pores sized to permit tissue ingrowth. The second microporous layer 9205 can be bonded to the third microporous layer 9205 to thereby form the reservoir 9100 for receiving the medicament. The first and second microporous materials 9201 and 9202 can be configured to meter the rate at which the medicament is dispensed from the reservoir 9100 when the delivery device is implanted.

According to principles of the present disclosure, the implantable medical system 9000 can be used to treat macular edema. In general, methods of treating diseases using an implantable medical system 9000 configured to meter a rate at which the medicament is dispensed from the reservoir 9100 when the delivery device is implanted are disclosed. The method can include selecting an implantable medical system 9000 that is similar to the implantable medical system 9000 discussed above. For instance, the first microporous material 9201 can have a first microporous layer 9203 that includes a plurality of pores sized to resist tissue ingrowth. The first microporous material 9201 can have a second microporous layer 9205 that includes a plurality of pores sized to permit tissue ingrowth. The second microporous material 9202 can have a third microporous layer 9205 that includes a plurality of pores sized to resist tissue ingrowth. The second microporous material 9202 can have a fourth microporous layer 9207 that includes a plurality of pores sized to permit tissue ingrowth. The second microporous layer 9205 can be bonded to the third microporous layer 9205 to thereby form a reservoir 9100 for receiving the medicament. The first and second microporous materials 9201 and 9202 can be configured to meter a rate at which the macular edema medicament is dispensed from the reservoir 9100 when the delivery device is implanted.

Administration of the medicament can include several steps. For instance, the method can include filling the reservoir 9100 with a medicament for treating macular edema. In certain instances, portions or all of the first and second microporous material 9202 can be elastomeric such that they reseal after insertion of a syringe 9110 or other similar devices. The method can include implanting the implantable medical system 9000 at an implantation location. The method can include allowing the medicament to dispense from the reservoir 9100 to one or more treatment sites.

FIGS. 11A-11D show various features of the reservoir 9100 in examples of medicament treatment devices. In particular, FIG. 11A shows the first configuration (Configuration “A”) of the reservoir 9100 where the reservoir 9100 includes the main portion 9101 only. FIG. 11B shows the second configuration (Configuration “B”) of the reservoir 9100 where the reservoir 9100 includes the main portion 9101 and the delivery arm 9103. FIG. 11C shows the third configuration (Configuration “C”) of the reservoir 9100 that is similar to that of FIG. 11A and further includes the plurality of chambers 9300 and the plurality of fill ports 9310 with which to fill the corresponding chamber with medicament. FIG. 11D shows the fourth configuration (Configuration “D”) of the reservoir 9100 that is similar to that of FIG. 11B and further includes the plurality of chambers 9300 and the plurality of fill ports 9310 with which to fill the corresponding chamber with medicament. In examples, fill ports 9310 can be an injection site at the reservoir 9100 or a feature of the implantable medical system 9000 that facilitates filling the reservoir 9100 with medicament. As further discussed below, the plurality of chambers 9300 (e.g., in FIGS. 11C and 11D) can be in fluid communication or fluidically isolated from one another. In examples, as further discussed elsewhere herein, the reservoir 9100 can dispense medicament in one direction (e.g., into the page) or in multiple directions (e.g., into the page, out of the page, in-plane with the page, or any combination thereof).

A number of factors can influence physical characteristics of medicament treatment devices. Depending on at least one of the implantation locations and the disease to be treated, one or both of a geometry and functionality of the reservoir 9100 of the medicament delivery system 1000 can vary in examples. For instance, dimensions the reservoir 9100 can be made larger or smaller when compared to other configurations of the medicament delivery system 1000. These variations can depend on, among other things, the amount of medicament to be delivered for treatment, the manner in which the medicament is to be delivered, and the resistance to delivery of medicament presented by an implantation location. As discussed here, in examples, the reservoir 9100 can have a generally uniform shape (e.g., resembling an ellipse, a circle, a polygon, etc.). In other examples, the reservoir 9100 can have an irregular shape (e.g., a generally uniform shape with one or more extrusions or protrusions therefrom, an eccentric shape, etc.). Some example shapes of the reservoir 9100 are discussed below with respect to certain example implementations of the medicament delivery system 1000. In examples, when the reservoir 9100 is inflated, the reservoir 9100 can bulge about along a length of the reservoir 9100. In some instances, bulging may cause the reservoir 9100 to balloon or to inflate to a generally polyhedron shape.

In certain examples, the physical characteristics of the reservoir 9100 can facilitate functionality of the medicament delivery system 1000. For instance, the reservoir 9100 can be formed to deliver medicament to a pinpoint location relative to the reservoir 9100, generally disperse medicament over a treatment site at one or more sides of the medicament delivery system 1000, and the like. In this regard, it can be said that the implantable medical system 9000 can have one-directional dispensing (e.g., at one side of the implantable medical system 9000) or multidirectional dispensing (e.g., at multiple sides of the implantable medical system 9000). In examples, one or more locations at the reservoir 9100 can function as the fill port 9310 with which to fill the reservoir 9100 with medicament. In this regard, the implantable medical system 9000 can be refillable with medicament as further discussed above. For example, the main portion 9101 of the reservoir 9100 can function as a refillable chamber, and the delivery arm 9103 can function as a conduit with which to deliver fluid from the refillable chamber. After implantation, medicament can be flushed or drained from the fill port 9310 at the reservoir 9100 to a treatment location. In examples, the reservoir 9100 can include the plurality of chambers 9300 therein with which to store medicament to be delivered. Under these circumstances, one chamber in the plurality of chambers 9300 can include a first medicament, and another chamber in the plurality of chambers 9300 can include a second medicament that is the same or different from the first medicament. Chambers 9300 in the plurality of chambers 9300 can be in fluid communication with one another or isolated from one another depending on the implementation.

Formed at the periphery of the reservoir 9100 can be the extended delivery arm 9103 through which medicament from the reservoir 9100 can be administered. In this regard, the reservoir 9100 can include the reservoir main portion 9101 and the extended delivery arm 9103 extending from the reservoir main portion 9101. For instance, the extended delivery arm 9103 can include a port through which medicament is exclusively allowed to exit the reservoir 9100. In other instances, medicament can be allowed exit the reservoir 9100 at any portion (e.g., not exclusively at the port) of the extended delivery arm 9103. In addition, or in alternative, medicament can be allowed to exit the reservoir 9100 both at the reservoir main portion 9101 and the extended delivery arm 9103.

FIGS. 12A-12P show various dispensing configurations of the reservoir 9100. In particular, FIGS. 12A-12H show the implantable medical system 9000 dispensing when in Configuration A of the reservoir 9100 where the reservoir 9100 includes the main portion 9101 only. FIGS. 12I-12P shows a Configuration B of the reservoir 9100 where the reservoir 9100 includes the main portion 9101 and the delivery arm 9103. Although for conciseness, the dispensing operations here are shown with respect to Configurations A and B, one skilled in the art will appreciate that these dispensing operations are similarly applicable to Configurations C and D. In this regard, the distinction would be that Configurations C and D include multiple chambers that may be similarly dispensed to the single chambers discussed below with respect to Configurations A and B. For illustration purposes, dispensing directions are shown as straight arrows flowing out of the reservoir 9100, and dispensing preventions are shown as curly arrows at the interior of the reservoir 9100. Dispensing can occur at only portions of the implantable medical system 9000 that are permeable, and dispensing preventions can occur at impermeable portions of the implantable medical system 9000. In addition, flow and directions into the page are shown as an encircled X, and directions out of the page are shown as encircled dots. Of course, these are merely schematic representations of a more complex phenomena but are used here for clarity of discussion.

As will be discussed below in further detail, dispensing of the medicament can vary across configurations and within configurations. For instance, as discussed above, the implantable medical system 9000 can be configured such that medicament is generally dispensed in one direction or in a plurality of directions. Under these circumstances, dispensing can occur at the main portion 9101, the delivery arm 9103, or both the main portion 9101 and the delivery arm 9103 of the implantable medical system 9000. In further under these circumstances, directionality of dispensing at the main portion 9101, the delivery arm 9103, or both the main portion 9101 and delivery arm 9103 of the implantable medical system 9000 can be the same or can be different depending on the example. The example shown and discussed in these figures are just some of many examples disclosed herein. One skilled in the art will appreciate this fact and recognize that, in reading this disclosure as a whole, certain variations and modifications of these examples are logical extensions of the examples discussed below.

With reference again to FIGS. 12A and 12B, a single direction dispensing is shown (generally into the page in FIG. 12A and downward in FIG. 12B). Distribution of dispensing is shown in a sprayed pattern where the directionality of the arrows generally deviates from one another but in the same general direction. In contrast, FIGS. 12C and 12D show a similar dispensing operation (generally into the page in FIG. 12B and downward dispensing in FIG. 12D) but in a more focused manner where the directionality of the arrows generally do not deviate from one another and are in the same direction. In FIGS. 12A through 12D, dispensing preventions are shown in a variety of other directions and locations except where dispensing is allowed.

Turning again to FIGS. 12E and 12F, a multidirectional dispensing operation is shown (e.g., generally into and out of the page in FIG. 12E and upward and downward in FIG. 12F). Distribution of dispensing is shown in a focused manner (similar to FIGS. 12C and 12D) in multiple directions. As is indicated by the number of arrows, dispensing out of the page and FIG. 12E and upward in FIG. 12F is less than dispensing into the page in FIG. 12E and downward in FIG. 12F. Although shown in the same focused manner of dispensing in multiple directions, it should be appreciated that some examples include dispensing in the sprayed manner in one direction and a focused manner in another direction. It should also be appreciated that dispensing may be the same amount in each direction of dispensing or may be different amounts in each direction of dispensing. As discussed above, an amount of dispensing can be proportional to permeability at the dispensing portion of the implantable medical system 9000. As previously described, FIGS. 12G and 12H show a similar dispensing operation (generally into and out of the page in FIG. 12G and upward and downward dispensing in FIG. 12H) but in a more sprayed manner (similar to FIGS. 12A and 12B) in multiple directions. In FIGS. 12E through 12G, dispensing preventions are shown in a variety of other directions and locations except where dispensing is allowed.

As noted above, FIGS. 12I-12P shows a Configuration B of the reservoir 9100 where the reservoir 9100 includes a main portion 9101 and a delivery arm 9103. Dispensing with respect to the main portion 9101 in these examples can be similar to those discussed above with reference to FIGS. 12A-12H. The main portion 9101 of the implantable medical system 9000 can be similar to those discussed with respect to FIGS. 12A-12H. The examples shown in FIGS. 12I-12P can, in addition or in alternative, include dispensing from the delivery arm 9103 as is further discussed below. While specific examples are discussed below, it should be appreciated that the skilled artisan would recognize many other examples and combinations thereof in light of this disclosure.

Turning to FIGS. 121 and 12J, a multidirectional dispensing operation at the delivery arm 9103 is shown. In this regard, dispensing at the delivery arm 9103 can occur in a variety of directions (e.g., at a tip portion of the delivery arm 9103 and along the length of the delivery arm 9103). Specific to this example, no dispensing occurs at the main portion 9101 of the implantable medical system 9000. In contrast, in the example shown in FIGS. 12M and 12N both the delivery arm 9103 and the main portion 9101 are configured to dispense in multiple directions. For instance, the main portion 9101 can dispense medicament in a similar manner as discussed with respect to FIGS. 12G and 12H, and the delivery arm 9103 can dispense medicament in a similar manner as discussed with respect to FIGS. 121 and 12J.

As previously discussed with reference to FIGS. 12K and 12L, a guided multidirectional dispensing operation at the delivery arm 9103 is shown. In this regard, dispensing at the delivery arm 9103 can occur in a variety of directions at only a tip portion of the delivery arm 9103. Specific to this example, no dispensing occurs at the main portion 9101 of the implantable medical system 9000 or along the length of the delivery arm 9103 separate from the tip portion. In contrast, in the example shown in FIGS. 12O and 12P the delivery arm 9103 is configured for guided multidirectional dispensing, and the main portion 9101 is configured for a focused dispensing. For instance, the main portion 9101 can dispense medicament in a similar manner as discussed with respect to FIGS. 12C and 12D, and the delivery arm 9103 can dispense medicament in a similar manner as discussed with respect to FIGS. 121 and 12J.

Any of the above configurations of the implantable medical system 100 and dispensing mechanisms may be applied for use in treating macular edema. Under these circumstances, the medicament delivery system (e.g., in Configurations A and C) can be arranged at the posterior location of the eye 5000 and optionally (e.g., in Configurations B and D) at the anterior location of the eye 5000. In some such circumstances, the medicament delivery system (e.g., in Configurations B and D) is positioned at the anterior location of the eye 5000 and arranged for intravitreal administration. API classes can include monoclonal antibodies such as ranibizumab, afibercept, LUCENTIS, and EYLEA. API classes can include steroids such as dexamethasone, fluocinolone acetonide, OZURDEX, or RETISERT. Drug delivery vehicles can be used during administration of APIs. For example, with respect to RETISERT, such drug delivery vehicles can include a silicone cup with poly(vinyl alcohol) (“PVA”) membrane, which can deliver the medicament over about 2-3 years. Delivery vehicles for OZURDEX can include a poly(lactic-co-glycolic acid) (“PLGA”) matrix with the drug. API classes can also include small binding molecules defined as antagonist molecules that enable inhibition of angiogenic growth factors, including VEGF or PDGF, or inhibit protein kinases. In further examples, API classes can include antibody mimetic proteins and peptides.

### Retinitis

Another retinal disease that can be treated using example implementations of the implantable medical system 9000 is retinitis. Retinitis is a disease of the eye 5000 that involves inflammation of the retina. For treatment of retinitis, a medicament delivery system can be suprachoroidally implanted (e.g., posterior of the pars plana of the eye 5000). In examples, the medicament delivery system can be any of Configurations A-D. For example, the medicament delivery system can be the implantable medical system 9000 as described previously throughout with reference to FIGS. 10A-12P and described again herein. These implantable medical systems 9000 can be similar to others disclosed elsewhere herein, including the medicament delivery systems 1000, 2000, 3000. In this regard, an implantable medical system 9000 can be implanted and arranged at one or more implantation locations or sites to deliver medicament for treatment of retinitis.

As previously described, FIGS. 10 and 10A-10D show several examples of an implantable medical system 9000 that is useful for employing these aspects of the present disclosure and may be used for treating retinitis. In particular, FIG. 10 is an isometric view of the implantable medical system 9000 having the reservoir 9100 with the main portion 9101 and the delivery arm 9103. FIG. 10A shows the inflated reservoir 9100 with FIGS. 10A-1 and 10A-2 therein showing a microscopic view of a first and second microporous material 9202 respectively in the implantable medical system 9000. FIG. 10B shows the reservoir 9100 in FIG. 10A being refilled via insertion of the syringe 9110 (although other suitable means for refilling the reservoir 9100 in situ may be used). In particular, FIG. 10C shows the inflated reservoir 9100 with the delivery arm 9103. FIGS. 10C-1 and 10C-2 therein showing a microscopic view of a first and second microporous material 9202 respectively in the implantable medical system 9000. FIG. 10D shows the reservoir 9100 in FIG. 10C being refilled via insertion of the syringe 9110 (although other suitable means for refilling the reservoir 9100 in situ may be used). In examples, the main portion 9101 and the delivery arm 9103 can comprise the same or similar materials. Under these circumstances, the delivery arm 9103 can be made integral with or attachable to the main portion 9101 such that the delivery arm 9103 and the main portion 9101 are in fluid communication with each other. The example microporous material is shown as a schematic in FIGS. 10A-1 and 10A-2 for clarity and illustration purposes. The illustrative imagery provided in FIGS. 10A-1, 10A-2, 10C-1, and 10C-2 are representative of the microscopic imagery presented in FIG. 18. Further discussion of microporous materials that are appropriate for employing principles of the present disclosure are shown and described in U.S. Pat. Pub. US2018/0263817, the entire contents of which are hereby incorporated by reference.

The medicant delivery device shown here in FIGS. 10A-10D can be similar to the implantable delivery devices 9000 discussed above. For instance, this device can be used to dispense medicament. The implantable medical system 9000 can include the material structure presented in FIGS. 10A-1 and 10C-1 and shown in FIG. 18. The implantable medical system 9000 can also include the material structure, selective permeability, and porous materials shown and described in U.S. Pat. No. 10,849,731 to Cully and as shown and described in U.S. Pat. Publ. No. 2018/0126134 to Cully, both of which are incorporated by reference in their entireties, with particular reference to FIGS. 3-10 and the accompanying descriptions provided in each of those incorporated references. The implantable medical system 9000 can further include the first microporous material 9201 that is bonded to the second microporous material 9202. The first microporous material 9201 can have the first microporous layer 9203 that includes the plurality of pores sized to permit tissue ingrowth. The first microporous material 9201 can have the second microporous layer 9205 that includes the plurality of pores sized to permit tissue ingrowth. The second microporous material 9202 can have the third microporous layer 9205 that includes the plurality of pores sized to resist tissue ingrowth. The second microporous material 9202 can have the fourth microporous layer 9207 that includes the plurality of pores sized to permit tissue ingrowth. The second microporous layer 9205 can be bonded to the third microporous layer 9205 to thereby form the reservoir 9100 for receiving the medicament. The first and second microporous materials 9201 and 9202 can be configured to meter the rate at which the medicament is dispensed from the reservoir 9100 when the delivery device is implanted.

According to principles of the present disclosure, the implantable medical system 9000 can be used to treat retinitis. In general, methods of treating diseases using an implantable medical system 9000 configured to meter a rate at which the medicament is dispensed from the reservoir 9100 when the delivery device is implanted are disclosed. The method can include selecting an implantable medical system 9000 that is similar to the implantable medical system 9000 discussed above. For instance, the first microporous material 9201 can have a first microporous layer 9203 that includes a plurality of pores sized to resist tissue ingrowth. The first microporous material 9201 can have a second microporous layer 9205 that includes a plurality of pores sized to permit tissue ingrowth. The second microporous material 9202 can have a third microporous layer 9205 that includes a plurality of pores sized to resist tissue ingrowth. The second microporous material 9202 can have a fourth microporous layer 9207 that includes a plurality of pores sized to permit tissue ingrowth. The second microporous layer 9205 can be bonded to the third microporous layer 9205 to thereby form a reservoir 9100 for receiving the medicament. The first and second microporous materials 9201 and 9202 can be configured to meter a rate at which the retinitis medicament is dispensed from the reservoir 9100 when the delivery device is implanted.

Administration of the medicament can include several steps. For instance, the method can include filling the reservoir 9100 with a medicament for treating retinitis. In certain instances, portions or all of the first and second microporous material 9202 can be elastomeric such that they reseal after insertion of a syringe 9110 or other similar devices. The method can include implanting the implantable medical system 9000 at an implantation location. The method can include allowing the medicament to dispense from the reservoir 9100 to one or more treatment sites.

FIGS. 11A-11D show various features of the reservoir 9100 in examples of medicament treatment devices. In particular, FIG. 11A shows the first configuration (Configuration “A”) of the reservoir 9100 where the reservoir 9100 includes the main portion 9101 only. FIG. 11B shows the second configuration (Configuration “B”) of the reservoir 9100 where the reservoir 9100 includes the main portion 9101 and the delivery arm 9103. FIG. 11C shows the third configuration (Configuration “C”) of the reservoir 9100 that is similar to that of FIG. 11A and further includes the plurality of chambers 9300 and the plurality of fill ports 9310 with which to fill the corresponding chamber with medicament. FIG. 11D shows the fourth configuration (Configuration “D”) of the reservoir 9100 that is similar to that of FIG. 11B and further includes the plurality of chambers 9300 and the plurality of fill ports 9310 with which to fill the corresponding chamber with medicament. In examples, fill ports 9310 can be an injection site at the reservoir 9100 or a feature of the implantable medical system 9000 that facilitates filling the reservoir 9100 with medicament. As further discussed below, the plurality of chambers 9300 (e.g., in FIGS. 11C and 11D) can be in fluid communication or fluidically isolated from one another. In examples, as further discussed elsewhere herein, the reservoir 9100 can dispense medicament in one direction (e.g., into the page) or in multiple directions (e.g., into the page, out of the page, in-plane with the page, or any combination thereof).

A number of factors can influence physical characteristics of medicament treatment devices. Depending on at least one of the implantation locations and the disease to be treated, one or both of a geometry and functionality of the reservoir 9100 of the medicament delivery system 1000 can vary in examples. For instance, dimensions the reservoir 9100 can be made larger or smaller when compared to other configurations of the medicament delivery system 1000. These variations can depend on, among other things, the amount of medicament to be delivered for treatment, the manner in which the medicament is to be delivered, and the resistance to delivery of medicament presented by an implantation location. As discussed here, in examples, the reservoir 9100 can have a generally uniform shape (e.g., resembling an ellipse, a circle, a polygon, etc.). In other examples, the reservoir 9100 can have an irregular shape (e.g., a generally uniform shape with one or more extrusions or protrusions therefrom, an eccentric shape, etc.). Some example shapes of the reservoir 9100 are discussed below with respect to certain example implementations of the medicament delivery system 1000. In examples, when the reservoir 9100 is inflated, the reservoir 9100 can bulge about along a length of the reservoir 9100. In some instances, bulging may cause the reservoir 9100 to balloon or to inflate to a generally polyhedron shape.

In certain examples, the physical characteristics of the reservoir 9100 can facilitate functionality of the medicament delivery system 1000. For instance, the reservoir 9100 can be formed to deliver medicament to a pinpoint location relative to the reservoir 9100, generally disperse medicament over a treatment site at one or more sides of the medicament delivery system 1000, and the like. In this regard, it can be said that the implantable medical system 9000 can have one-directional dispensing (e.g., at one side of the implantable medical system 9000) or multidirectional dispensing (e.g., at multiple sides of the implantable medical system 9000). In examples, one or more locations at the reservoir 9100 can function as the fill port 9310 with which to fill the reservoir 9100 with medicament. In this regard, the implantable medical system 9000 can be refillable with medicament as further discussed above. For example, the main portion 9101 of the reservoir 9100 can function as a refillable chamber, and the delivery arm 9103 can function as a conduit with which to deliver fluid from the refillable chamber. After implantation, medicament can be flushed or drained from the fill port 9310 at the reservoir 9100 to a treatment location. In examples, the reservoir 9100 can include the plurality of chambers 9300 therein with which to store medicament to be delivered. Under these circumstances, one chamber in the plurality of chambers 9300 can include a first medicament, and another chamber in the plurality of chambers 9300 can include a second medicament that is the same or different from the first medicament. Chambers 9300 in the plurality of chambers 9300 can be in fluid communication with one another or isolated from one another depending on the implementation.

Formed at the periphery of the reservoir 9100 can be the extended delivery arm 9103 through which medicament from the reservoir 9100 can be administered. In this regard, the reservoir 9100 can include the reservoir main portion 9101 and the extended delivery arm 9103 extending from the reservoir main portion 9101. For instance, the extended delivery arm 9103 can include a port through which medicament is exclusively allowed to exit the reservoir 9100. In other instances, medicament can be allowed exit the reservoir 9100 at any portion (e.g., not exclusively at the port) of the extended delivery arm 9103. In addition, or in alternative, medicament can be allowed to exit the reservoir 9100 both at the reservoir main portion 9101 and the extended delivery arm 9103.

FIGS. 12A-12P show various dispensing configurations of the reservoir 9100. In particular, FIGS. 12A-12H show the implantable medical system 9000 dispensing when in Configuration A of the reservoir 9100 where the reservoir 9100 includes the main portion 9101 only. FIGS. 12I-12P shows a Configuration B of the reservoir 9100 where the reservoir 9100 includes the main portion 9101 and the delivery arm 9103. Although for conciseness, the dispensing operations here are shown with respect to Configurations A and B, one skilled in the art will appreciate that these dispensing operations are similarly applicable to Configurations C and D. In this regard, the distinction would be that Configurations C and D include multiple chambers that may be similarly dispensed to the single chambers discussed below with respect to Configurations A and B. For illustration purposes, dispensing directions are shown as straight arrows flowing out of the reservoir 9100, and dispensing preventions are shown as curly arrows at the interior of the reservoir 9100. Dispensing can occur at only portions of the implantable medical system 9000 that are permeable, and dispensing preventions can occur at impermeable portions of the implantable medical system 9000. In addition, flow and directions into the page are shown as an encircled X, and directions out of the page are shown as encircled dots. Of course, these are merely schematic representations of a more complex phenomena but are used here for clarity of discussion.

As will be discussed below in further detail, dispensing of the medicament can vary across configurations and within configurations. For instance, as discussed above, the implantable medical system 9000 can be configured such that medicament is generally dispensed in one direction or in a plurality of directions. Under these circumstances, dispensing can occur at the main portion 9101, the delivery arm 9103, or both the main portion 9101 and the delivery arm 9103 of the implantable medical system 9000. In further under these circumstances, directionality of dispensing at the main portion 9101, the delivery arm 9103, or both the main portion 9101 and delivery arm 9103 of the implantable medical system 9000 can be the same or can be different depending on the example. The example shown and discussed in these figures are just some of many examples disclosed herein. One skilled in the art will appreciate this fact and recognize that, in reading this disclosure as a whole, certain variations and modifications of these examples are logical extensions of the examples discussed below.

With reference again to FIGS. 12A and 12B, a single direction dispensing is shown (generally into the page in FIG. 12A and downward in FIG. 12B). Distribution of dispensing is shown in a sprayed pattern where the directionality of the arrows generally deviates from one another but in the same general direction. In contrast, FIGS. 12C and 12D show a similar dispensing operation (generally into the page in FIG. 12B and downward dispensing in FIG. 12D) but in a more focused manner where the directionality of the arrows generally do not deviate from one another and are in the same direction. In FIGS. 12A through 12D, dispensing preventions are shown in a variety of other directions and locations except where dispensing is allowed.

Turning again to FIGS. 12E and 12F, a multidirectional dispensing operation is shown (e.g., generally into and out of the page in FIG. 12E and upward and downward in FIG. 12F). Distribution of dispensing is shown in a focused manner (similar to FIGS. 12C and 12D) in multiple directions. As is indicated by the number of arrows, dispensing out of the page and FIG. 12E and upward in FIG. 12F is less than dispensing into the page in FIG. 12E and downward in FIG. 12F. Although shown in the same focused manner of dispensing in multiple directions, it should be appreciated that some examples include dispensing in the sprayed manner in one direction and a focused manner in another direction. It should also be appreciated that dispensing may be the same amount in each direction of dispensing or may be different amounts in each direction of dispensing. As discussed above, an amount of dispensing can be proportional to permeability at the dispensing portion of the implantable medical system 9000. As previously described, FIGS. 12G and 12H show a similar dispensing operation (generally into and out of the page in FIG. 12G and upward and downward dispensing in FIG. 12H) but in a more sprayed manner (similar to FIGS. 12A and 12B) in multiple directions. In FIGS. 12E through 12G, dispensing preventions are shown in a variety of other directions and locations except where dispensing is allowed.

As noted above, FIGS. 12I-12P shows a Configuration B of the reservoir 9100 where the reservoir 9100 includes a main portion 9101 and a delivery arm 9103. Dispensing with respect to the main portion 9101 in these examples can be similar to those discussed above with reference to FIGS. 12A-12H. The main portion 9101 of the implantable medical system 9000 can be similar to those discussed with respect to FIGS. 12A-12H. The examples shown in FIGS. 12I-12P can, in addition or in alternative, include dispensing from the delivery arm 9103 as is further discussed below. While specific examples are discussed below, it should be appreciated that the skilled artisan would recognize many other examples and combinations thereof in light of this disclosure.

Turning to FIGS. 121 and 12J, a multidirectional dispensing operation at the delivery arm 9103 is shown. In this regard, dispensing at the delivery arm 9103 can occur in a variety of directions (e.g., at a tip portion of the delivery arm 9103 and along the length of the delivery arm 9103). Specific to this example, no dispensing occurs at the main portion 9101 of the implantable medical system 9000. In contrast, in the example shown in FIGS. 12M and 12N both the delivery arm 9103 and the main portion 9101 are configured to dispense in multiple directions. For instance, the main portion 9101 can dispense medicament in a similar manner as discussed with respect to FIGS. 12G and 12H, and the delivery arm 9103 can dispense medicament in a similar manner as discussed with respect to FIGS. 121 and 12J.

As previously discussed with reference to FIGS. 12K and 12L, a guided multidirectional dispensing operation at the delivery arm 9103 is shown. In this regard, dispensing at the delivery arm 9103 can occur in a variety of directions at only a tip portion of the delivery arm 9103. Specific to this example, no dispensing occurs at the main portion 9101 of the implantable medical system 9000 or along the length of the delivery arm 9103 separate from the tip portion. In contrast, in the example shown in FIGS. 12O and 12P the delivery arm 9103 is configured for guided multidirectional dispensing, and the main portion 9101 is configured for a focused dispensing. For instance, the main portion 9101 can dispense medicament in a similar manner as discussed with respect to FIGS. 12C and 12D, and the delivery arm 9103 can dispense medicament in a similar manner as discussed with respect to FIGS. 121 and 12J. Any of the above configurations of the implantable medical system 100 and dispensing mechanisms may be applied for use in treating retinitis.

Under these circumstances, the medicament delivery system (e.g., in Configurations A and C) can be arranged at the posterior location of the eye 5000 and optionally (e.g., in Configurations B and D) at the anterior location of the eye 5000. In some such circumstances, the medicament delivery system (e.g., in Configurations B and D) is positioned at the anterior location of the eye 5000 and arranged for intravitreal administration. API classes can include antibiotics and antivirals (for cytomegalovirus (“CMV”)) such as ganciclovir or VITRASERT. Drug delivery vehicles can be used during administration of APIs. For example, with respect to ganciclovir and VITRASERT, such drug delivery vehicles can include a silicone cup with PVA membrane, which can deliver the medicament over about 2-3 years.

### Retinoblastoma

Yet another retinal disease that can be treated using example implementations of the implantable medical system 9000 is retinoblastoma, which is a form of eye cancer. For treatment of retinoblastoma, a medicament delivery system can be suprachoroidally implanted (e.g., posterior of the pars plana of the eye 5000). In examples, the medicament delivery system can be any of Configurations A and C. For example, the medicament delivery system can be the implantable medical system 9000 as described previously throughout with reference to FIGS. 10A-12P and described again herein. These implantable medical systems 9000 can be similar to others disclosed elsewhere herein, including the medicament delivery systems 1000, 2000, 3000. In this regard, an implantable medical system 9000 can be implanted and arranged at one or more implantation locations or sites to deliver medicament for treatment of retinoblastoma.

As previously described, FIGS. 10 and 10A-10D show several examples of an implantable medical system 9000 that is useful for employing these aspects of the present disclosure. In particular, FIG. 10 is an isometric view of the implantable medical system 9000 having the reservoir 9100 with the main portion 9101 and the delivery arm 9103. FIG. 10A shows the inflated reservoir 9100 with FIGS. 10A-1 and 10A-2 therein showing a microscopic view of a first and second microporous material 9202 respectively in the implantable medical system 9000. FIG. 10B shows the reservoir 9100 in FIG. 10A being refilled via insertion of the syringe 9110 (although other suitable means for refilling the reservoir 9100 in situ may be used). In particular, FIG. 10C shows the inflated reservoir 9100 with the delivery arm 9103. FIGS. 10C-1 and 10C-2 therein showing a microscopic view of a first and second microporous material 9202 respectively in the implantable medical system 9000. FIG. 10D shows the reservoir 9100 in FIG. 10C being refilled via insertion of the syringe 9110 (although other suitable means for refilling the reservoir 9100 in situ may be used). In examples, the main portion 9101 and the delivery arm 9103 can comprise the same or similar materials. Under these circumstances, the delivery arm 9103 can be made integral with or attachable to the main portion 9101 such that the delivery arm 9103 and the main portion 9101 are in fluid communication with each other. The example microporous material is shown as a schematic in FIGS. 10A-1 and 10A-2 for clarity and illustration purposes. The illustrative imagery provided in FIGS. 10A-1, 10A-2, 10C-1, and 10C-2 are representative of the microscopic imagery presented in FIG. 18. Further discussion of microporous materials that are appropriate for employing principles of the present disclosure are shown and described in U.S. Pat. Pub. US2018/0263817, the entire contents of which are hereby incorporated by reference.

The medicant delivery device shown here in FIGS. 10A-10D can be similar to the implantable delivery devices 9000 discussed above. For instance, this device can be used to dispense medicament as discussed above. The implantable medical system 9000 can include the material structure presented in FIGS. 10A-1 and 10C-1 and shown in FIG. 18. The implantable medical system 9000 can also include the material structure, selective permeability, and porous materials shown and described in U.S. Pat. No. 10,849,731 to Cully and as shown and described in U.S. Pat. Publ. No. 2018/0126134 to Cully, both of which are incorporated by reference in their entireties, with particular reference to FIGS. 3-10 and the accompanying descriptions provided in each of those incorporated references. The implantable medical system 9000 can further include the first microporous material 9201 that is bonded to the second microporous material 9202. The first microporous material 9201 can have the first microporous layer 9203 that includes the plurality of pores sized to permit tissue ingrowth. The first microporous material 9201 can have the second microporous layer 9205 that includes the plurality of pores sized to permit tissue ingrowth. The second microporous material 9202 can have the third microporous layer 9205 that includes the plurality of pores sized to resist tissue ingrowth. The second microporous material 9202 can have the fourth microporous layer 9207 that includes the plurality of pores sized to permit tissue ingrowth. The second microporous layer 9205 can be bonded to the third microporous layer 9205 to thereby form the reservoir 9100 for receiving the medicament. The first and second microporous materials 9201 and 9202 can be configured to meter the rate at which the medicament is dispensed from the reservoir 9100 when the delivery device is implanted.

According to principles of the present disclosure, the implantable medical system 9000 can be used to treat retinoblastoma. In general, methods of treating diseases using an implantable medical system 9000 configured to meter a rate at which the medicament is dispensed from the reservoir 9100 when the delivery device is implanted are disclosed. The method can include selecting an implantable medical system 9000 that is similar to the implantable medical system 9000 discussed above. For instance, the first microporous material 9201 can have a first microporous layer 9203 that includes a plurality of pores sized to resist tissue ingrowth. The first microporous material 9201 can have a second microporous layer 9205 that includes a plurality of pores sized to permit tissue ingrowth. The second microporous material 9202 can have a third microporous layer 9205 that includes a plurality of pores sized to resist tissue ingrowth. The second microporous material 9202 can have a fourth microporous layer 9207 that includes a plurality of pores sized to permit tissue ingrowth. The second microporous layer 9205 can be bonded to the third microporous layer 9205 to thereby form a reservoir 9100 for receiving the medicament. The first and second microporous materials 9201 and 9202 can be configured to meter a rate at which the retinoblastoma medicament is dispensed from the reservoir 9100 when the delivery device is implanted.

Administration of the medicament can include several steps. For instance, the method can include filling the reservoir 9100 with a medicament for treating retinoblastoma. In certain instances, portions or all of the first and second microporous material 9202 can be elastomeric such that they reseal after insertion of a syringe 9110 or other similar devices. The method can include implanting the implantable medical system 9000 at an implantation location. The method can include allowing the medicament to dispense from the reservoir 9100 to one or more treatment sites.

FIGS. 11A and 11C show various features of the reservoir 9100 in examples of medicament treatment devices which may be used for treating retinoblastoma. In particular, FIG. 11A shows the first configuration (Configuration “A”) of the reservoir 9100 where the reservoir 9100 includes the main portion 9101 only. FIG. 11C shows the third configuration (Configuration “C”) of the reservoir 9100 that is similar to that of FIG. 11A and further includes the plurality of chambers 9300 and the plurality of fill ports 9310 with which to fill the corresponding chamber with medicament.

A number of factors can influence physical characteristics of medicament treatment devices. Depending on at least one of the implantation locations and the disease to be treated, one or both of a geometry and functionality of the reservoir 9100 of the medicament delivery system 1000 can vary in examples. For instance, dimensions the reservoir 9100 can be made larger or smaller when compared to other configurations of the medicament delivery system 1000. These variations can depend on, among other things, the amount of medicament to be delivered for treatment, the manner in which the medicament is to be delivered, and the resistance to delivery of medicament presented by an implantation location. As discussed here, in examples, the reservoir 9100 can have a generally uniform shape (e.g., resembling an ellipse, a circle, a polygon, etc.). In other examples, the reservoir 9100 can have an irregular shape (e.g., a generally uniform shape with one or more extrusions or protrusions therefrom, an eccentric shape, etc.). Some example shapes of the reservoir 9100 are discussed below with respect to certain example implementations of the medicament delivery system 1000. In examples, when the reservoir 9100 is inflated, the reservoir 9100 can bulge about along a length of the reservoir 9100. In some instances, bulging may cause the reservoir 9100 to balloon or to inflate to a generally polyhedron shape.

In certain examples, the physical characteristics of the reservoir 9100 can facilitate functionality of the medicament delivery system 1000. For instance, the reservoir 9100 can be formed to deliver medicament to a pinpoint location relative to the reservoir 9100, generally disperse medicament over a treatment site at one or more sides of the medicament delivery system 1000, and the like. In this regard, it can be said that the implantable medical system 9000 can have one-directional dispensing (e.g., at one side of the implantable medical system 9000) or multidirectional dispensing (e.g., at multiple sides of the implantable medical system 9000). In examples, one or more locations at the reservoir 9100 can function as the fill port 9310 with which to fill the reservoir 9100 with medicament. In this regard, the implantable medical system 9000 can be refillable with medicament as further discussed above. For example, the main portion 9101 of the reservoir 9100 can function as a refillable chamber, and the delivery arm 9103 can function as a conduit with which to deliver fluid from the refillable chamber. After implantation, medicament can be flushed or drained from the fill port 9310 at the reservoir 9100 to a treatment location. In examples, the reservoir 9100 can include the plurality of chambers 9300 therein with which to store medicament to be delivered. Under these circumstances, one chamber in the plurality of chambers 9300 can include a first medicament, and another chamber in the plurality of chambers 9300 can include a second medicament that is the same or different from the first medicament. Chambers 9300 in the plurality of chambers 9300 can be in fluid communication with one another or isolated from one another depending on the implementation.

Formed at the periphery of the reservoir 9100 can be the extended delivery arm 9103 through which medicament from the reservoir 9100 can be administered. In this regard, the reservoir 9100 can include the reservoir main portion 9101 and the extended delivery arm 9103 extending from the reservoir main portion 9101. For instance, the extended delivery arm 9103 can include a port through which medicament is exclusively allowed to exit the reservoir 9100. In other instances, medicament can be allowed exit the reservoir 9100 at any portion (e.g., not exclusively at the port) of the extended delivery arm 9103. In addition, or in alternative, medicament can be allowed to exit the reservoir 9100 both at the reservoir main portion 9101 and the extended delivery arm 9103.

FIGS. 12A-12P show various dispensing configurations of the reservoir 9100. In particular, FIGS. 12A-12H show the implantable medical system 9000 dispensing when in Configuration A of the reservoir 9100 where the reservoir 9100 includes the main portion 9101 only. Although for conciseness, the dispensing operations described are shown with respect to Configuration A, one skilled in the art will appreciate that these dispensing operations are similarly applicable to Configuration C. In this regard, the distinction would be that Configuration C includes multiple chambers that may be similarly dispensed to the single chambers discussed below with respect to Configuration A. For illustration purposes, dispensing directions are shown as straight arrows flowing out of the reservoir 9100, and dispensing preventions are shown as curly arrows at the interior of the reservoir 9100. Dispensing can occur at only portions of the implantable medical system 9000 that are permeable, and dispensing preventions can occur at impermeable portions of the implantable medical system 9000. In addition, flow and directions into the page are shown as an encircled X, and directions out of the page are shown as encircled dots. Of course, these are merely schematic representations of a more complex phenomena but are used here for clarity of discussion.

As will be discussed below in further detail, dispensing of the medicament can vary across configurations and within configurations. For instance, as discussed above, the implantable medical system 9000 can be configured such that medicament is generally dispensed in one direction or in a plurality of directions. Under these circumstances, dispensing can occur at the main portion 9101. The example shown and discussed in these figures are just some of many examples disclosed herein. One skilled in the art will appreciate this fact and recognize that, in reading this disclosure as a whole, certain variations and modifications of these examples are logical extensions of the examples discussed below.

With reference again to FIGS. 12A and 12B, a single direction dispensing is shown (generally into the page in FIG. 12A and downward in FIG. 12B). Distribution of dispensing is shown in a sprayed pattern where the directionality of the arrows generally deviates from one another but in the same general direction. In contrast, FIGS. 12C and 12D show a similar dispensing operation (generally into the page in FIG. 12B and downward dispensing in FIG. 12D) but in a more focused manner where the directionality of the arrows generally do not deviate from one another and are in the same direction. In FIGS. 12A through 12D, dispensing preventions are shown in a variety of other directions and locations except where dispensing is allowed.

Turning again to FIGS. 12E and 12F, a multidirectional dispensing operation is shown (e.g., generally into and out of the page in FIG. 12E and upward and downward in FIG. 12F). Distribution of dispensing is shown in a focused manner (similar to FIGS. 12C and 12D) in multiple directions. As is indicated by the number of arrows, dispensing out of the page and FIG. 12E and upward in FIG. 12F is less than dispensing into the page in FIG. 12E and downward in FIG. 12F. Although shown in the same focused manner of dispensing in multiple directions, it should be appreciated that some examples include dispensing in the sprayed manner in one direction and a focused manner in another direction. It should also be appreciated that dispensing may be the same amount in each direction of dispensing or may be different amounts in each direction of dispensing. As discussed above, an amount of dispensing can be proportional to permeability at the dispensing portion of the implantable medical system 9000. As previously described, FIGS. 12G and 12H show a similar dispensing operation (generally into and out of the page in FIG. 12G and upward and downward dispensing in FIG. 12H) but in a more sprayed manner (similar to FIGS. 12A and 12B) in multiple directions. In FIGS. 12E through 12G, dispensing preventions are shown in a variety of other directions and locations except where dispensing is allowed.

Any of the above configurations of the implantable medical system 100 and dispensing mechanisms may be applied for use in treating retinoblastoma. Under these circumstances, the medicament delivery system (e.g., in Configurations A and C) can be arranged at the posterior location of the eye 5000. API classes can include cytotoxic chemotherapy compounds such as vincristine or ONCOVIN.

### Retinal Vein Occlusions

Retinal vein occlusions are retinal diseases that can be treated using example implementations of the implantable medical system 9000. Retinal vein occlusions, such as CRVO and BRVO, are blockages of one or more retinal veins. These blockages can lead to excess blood and fluid in the retina. For treatment of retinal vein occlusions, a medicament delivery system can be suprachoroidially implanted (e.g., posterior of the pars plana of the eye 5000). In examples, the medicament delivery system can be any of Configurations A-D. For example, the medicament delivery system can be the implantable medical system 9000 as described previously throughout with reference to FIGS. 10A-12P and described again herein. These implantable medical systems 9000 can be similar to others disclosed elsewhere herein, including the medicament delivery systems 1000, 2000, 3000. In this regard, an implantable medical system 9000 can be implanted and arranged at one or more implantation locations or sites to deliver medicament for treatment of retinal vein occlusions.

As previously described, FIGS. 10 and 10A-10D show several examples of an implantable medical system 9000 that is useful for employing these aspects of the present disclosure. In particular, FIG. 10 is an isometric view of the implantable medical system 9000 having the reservoir 9100 with the main portion 9101 and the delivery arm 9103. FIG. 10A shows the inflated reservoir 9100 with FIGS. 10A-1 and 10A-2 therein showing a microscopic view of a first and second microporous material 9202 respectively in the implantable medical system 9000. FIG. 10B shows the reservoir 9100 in FIG. 10A being refilled via insertion of the syringe 9110 (although other suitable means for refilling the reservoir 9100 in situ may be used). In particular, FIG. 10C shows the inflated reservoir 9100 with the delivery arm 9103. FIGS. 10C-1 and 10C-2 therein showing a microscopic view of a first and second microporous material 9202 respectively in the implantable medical system 9000. FIG. 10D shows the reservoir 9100 in FIG. 10C being refilled via insertion of the syringe 9110 (although other suitable means for refilling the reservoir 9100 in situ may be used). In examples, the main portion 9101 and the delivery arm 9103 can comprise the same or similar materials. Under these circumstances, the delivery arm 9103 can be made integral with or attachable to the main portion 9101 such that the delivery arm 9103 and the main portion 9101 are in fluid communication with each other. The example microporous material is shown as a schematic in FIGS. 10A-1 and 10A-2 for clarity and illustration purposes. The illustrative imagery provided in FIGS. 10A-1, 10A-2, 10C-1 and 10C-2 are representative of the microscopic imagery presented in FIG. 18. Further discussion of microporous materials that are appropriate for employing principles of the present disclosure are shown and described in U.S. Pat. Pub. US2018/0263817, the entire contents of which are hereby incorporated by reference.

The medicant delivery device shown here in FIGS. 10A-10D can be similar to the implantable delivery devices 9000 discussed above. For instance, this device can be used to dispense medicament as discussed above. The implantable medical system 9000 can include the material structure presented in FIGS. 10A-1 and 10C-1 and shown in FIG. 18. The implantable medical system 9000 can also include the material structure, selective permeability, and porous materials shown and described in U.S. Pat. No. 10,849,731 to Cully and as shown and described in U.S. Pat. Publ. No. 2018/0126134 to Cully, both of which are incorporated by reference in their entireties, with particular reference to FIGS. 3-10 and the accompanying descriptions provided in each of those incorporated references. The implantable medical system 9000 can further include the first microporous material 9201 that is bonded to the second microporous material 9202. The first microporous material 9201 can have the first microporous layer 9203 that includes the plurality of pores sized to permit tissue ingrowth. The first microporous material 9201 can have the second microporous layer 9205 that includes the plurality of pores sized to permit tissue ingrowth. The second microporous material 9202 can have the third microporous layer 9205 that includes the plurality of pores sized to resist tissue ingrowth. The second microporous material 9202 can have the fourth microporous layer 9207 that includes the plurality of pores sized to permit tissue ingrowth. The second microporous layer 9205 can be bonded to the third microporous layer 9205 to thereby form the reservoir 9100 for receiving the medicament. The first and second microporous materials 9201 and 9202 can be configured to meter the rate at which the medicament is dispensed from the reservoir 9100 when the delivery device is implanted.

According to principles of the present disclosure, the implantable medical system 9000 can be used to treat retinal vein occlusions. In general, methods of treating diseases using an implantable medical system 9000 configured to meter a rate at which the medicament is dispensed from the reservoir 9100 when the delivery device is implanted are disclosed. The method can include selecting an implantable medical system 9000 that is similar to the implantable medical system 9000 discussed above. For instance, the first microporous material 9201 can have a first microporous layer 9203 that includes a plurality of pores sized to resist tissue ingrowth. The first microporous material 9201 can have a second microporous layer 9205 that includes a plurality of pores sized to permit tissue ingrowth. The second microporous material 9202 can have a third microporous layer 9205 that includes a plurality of pores sized to resist tissue ingrowth. The second microporous material 9202 can have a fourth microporous layer 9207 that includes a plurality of pores sized to permit tissue ingrowth. The second microporous layer 9205 can be bonded to the third microporous layer 9205 to thereby form a reservoir 9100 for receiving the medicament. The first and second microporous materials 9201 and 9202 can be configured to meter a rate at which the retinal vein occlusions medicament is dispensed from the reservoir 9100 when the delivery device is implanted.

Administration of the medicament can include several steps. For instance, the method can include filling the reservoir 9100 with a medicament for treating retinal vein occlusions. In certain instances, portions or all of the first and second microporous material 9202 can be elastomeric such that they reseal after insertion of a syringe 9110 or other similar devices. The method can include implanting the implantable medical system 9000 at an implantation location. The method can include allowing the medicament to dispense from the reservoir 9100 to one or more treatment sites.

FIGS. 11A-11D show various features of the reservoir 9100 in examples of medicament treatment devices. In particular, FIG. 11A shows the first configuration (Configuration “A”) of the reservoir 9100 where the reservoir 9100 includes the main portion 9101 only. FIG. 11B shows the second configuration (Configuration “B”) of the reservoir 9100 where the reservoir 9100 includes the main portion 9101 and the delivery arm 9103. FIG. 11C shows the third configuration (Configuration “C”) of the reservoir 9100 that is similar to that of FIG. 11A and further includes the plurality of chambers 9300 and the plurality of fill ports 9310 with which to fill the corresponding chamber with medicament. FIG. 11D shows the fourth configuration (Configuration “D”) of the reservoir 9100 that is similar to that of FIG. 11B and further includes the plurality of chambers 9300 and the plurality of fill ports 9310 with which to fill the corresponding chamber with medicament. In examples, fill ports 9310 can be an injection site at the reservoir 9100 or a feature of the implantable medical system 9000 that facilitates filling the reservoir 9100 with medicament. As further discussed below, the plurality of chambers 9300 (e.g., in FIGS. 11C and 11D) can be in fluid communication or fluidically isolated from one another. In examples, as further discussed elsewhere herein, the reservoir 9100 can dispense medicament in one direction (e.g., into the page) or in multiple directions (e.g., into the page, out of the page, in-plane with the page, or any combination thereof).

A number of factors can influence physical characteristics of medicament treatment devices. Depending on at least one of the implantation locations and the disease to be treated, one or both of a geometry and functionality of the reservoir 9100 of the medicament delivery system 1000 can vary in examples. For instance, dimensions the reservoir 9100 can be made larger or smaller when compared to other configurations of the medicament delivery system 1000. These variations can depend on, among other things, the amount of medicament to be delivered for treatment, the manner in which the medicament is to be delivered, and the resistance to delivery of medicament presented by an implantation location. As discussed here, in examples, the reservoir 9100 can have a generally uniform shape (e.g., resembling an ellipse, a circle, a polygon, etc.). In other examples, the reservoir 9100 can have an irregular shape (e.g., a generally uniform shape with one or more extrusions or protrusions therefrom, an eccentric shape, etc.). Some example shapes of the reservoir 9100 are discussed below with respect to certain example implementations of the medicament delivery system 1000. In examples, when the reservoir 9100 is inflated, the reservoir 9100 can bulge about along a length of the reservoir 9100. In some instances, bulging may cause the reservoir 9100 to balloon or to inflate to a generally polyhedron shape.

In certain examples, the physical characteristics of the reservoir 9100 can facilitate functionality of the medicament delivery system 1000. For instance, the reservoir 9100 can be formed to deliver medicament to a pinpoint location relative to the reservoir 9100, generally disperse medicament over a treatment site at one or more sides of the medicament delivery system 1000, and the like. In this regard, it can be said that the implantable medical system 9000 can have one-directional dispensing (e.g., at one side of the implantable medical system 9000) or multidirectional dispensing (e.g., at multiple sides of the implantable medical system 9000). In examples, one or more locations at the reservoir 9100 can function as the fill port 9310 with which to fill the reservoir 9100 with medicament. In this regard, the implantable medical system 9000 can be refillable with medicament as further discussed above. For example, the main portion 9101 of the reservoir 9100 can function as a refillable chamber, and the delivery arm 9103 can function as a conduit with which to deliver fluid from the refillable chamber. After implantation, medicament can be flushed or drained from the fill port 9310 at the reservoir 9100 to a treatment location. In examples, the reservoir 9100 can include the plurality of chambers 9300 therein with which to store medicament to be delivered. Under these circumstances, one chamber in the plurality of chambers 9300 can include a first medicament, and another chamber in the plurality of chambers 9300 can include a second medicament that is the same or different from the first medicament. Chambers 9300 in the plurality of chambers 9300 can be in fluid communication with one another or isolated from one another depending on the implementation.

Formed at the periphery of the reservoir 9100 can be the extended delivery arm 9103 through which medicament from the reservoir 9100 can be administered. In this regard, the reservoir 9100 can include the reservoir main portion 9101 and the extended delivery arm 9103 extending from the reservoir main portion 9101. For instance, the extended delivery arm 9103 can include a port through which medicament is exclusively allowed to exit the reservoir 9100. In other instances, medicament can be allowed exit the reservoir 9100 at any portion (e.g., not exclusively at the port) of the extended delivery arm 9103. In addition, or in alternative, medicament can be allowed to exit the reservoir 9100 both at the reservoir main portion 9101 and the extended delivery arm 9103.

FIGS. 12A-12P show various dispensing configurations of the reservoir 9100. In particular, FIGS. 12A-12H show the implantable medical system 9000 dispensing when in Configuration A of the reservoir 9100 where the reservoir 9100 includes the main portion 9101 only. FIGS. 12I-12P shows a Configuration B of the reservoir 9100 where the reservoir 9100 includes the main portion 9101 and the delivery arm 9103. Although for conciseness, the dispensing operations here are shown with respect to Configurations A and B, one skilled in the art will appreciate that these dispensing operations are similarly applicable to Configurations C and D. In this regard, the distinction would be that Configurations C and D include multiple chambers that may be similarly dispensed to the single chambers discussed below with respect to Configurations A and B. For illustration purposes, dispensing directions are shown as straight arrows flowing out of the reservoir 9100, and dispensing preventions are shown as curly arrows at the interior of the reservoir 9100. Dispensing can occur at only portions of the implantable medical system 9000 that are permeable, and dispensing preventions can occur at impermeable portions of the implantable medical system 9000. In addition, flow and directions into the page are shown as an encircled X, and directions out of the page are shown as encircled dots. Of course, these are merely schematic representations of a more complex phenomena but are used here for clarity of discussion.

As will be discussed below in further detail, dispensing of the medicament can vary across configurations and within configurations. For instance, as discussed above, the implantable medical system 9000 can be configured such that medicament is generally dispensed in one direction or in a plurality of directions. Under these circumstances, dispensing can occur at the main portion 9101, the delivery arm 9103, or both the main portion 9101 and the delivery arm 9103 of the implantable medical system 9000. In further under these circumstances, directionality of dispensing at the main portion 9101, the delivery arm 9103, or both the main portion 9101 and delivery arm 9103 of the implantable medical system 9000 can be the same or can be different depending on the example. The example shown and discussed in these figures are just some of many examples disclosed herein. One skilled in the art will appreciate this fact and recognize that, in reading this disclosure as a whole, certain variations and modifications of these examples are logical extensions of the examples discussed below.

With reference again to FIGS. 12A and 12B, a single direction dispensing is shown (generally into the page in FIG. 12A and downward in FIG. 12B). Distribution of dispensing is shown in a sprayed pattern where the directionality of the arrows generally deviates from one another but in the same general direction. In contrast, FIGS. 12C and 12D show a similar dispensing operation (generally into the page in FIG. 12B and downward dispensing in FIG. 12D) but in a more focused manner where the directionality of the arrows generally do not deviate from one another and are in the same direction. In FIGS. 12A through 12D, dispensing preventions are shown in a variety of other directions and locations except where dispensing is allowed.

Turning again to FIGS. 12E and 12F, a multidirectional dispensing operation is shown (e.g., generally into and out of the page in FIG. 12E and upward and downward in FIG. 12F). Distribution of dispensing is shown in a focused manner (similar to FIGS. 12C and 12D) in multiple directions. As is indicated by the number of arrows, dispensing out of the page and FIG. 12E and upward in FIG. 12F is less than dispensing into the page in FIG. 12E and downward in FIG. 12F. Although shown in the same focused manner of dispensing in multiple directions, it should be appreciated that some examples include dispensing in the sprayed manner in one direction and a focused manner in another direction. It should also be appreciated that dispensing may be the same amount in each direction of dispensing or may be different amounts in each direction of dispensing. As discussed above, an amount of dispensing can be proportional to permeability at the dispensing portion of the implantable medical system 9000. As previously described, FIGS. 12G and 12H show a similar dispensing operation (generally into and out of the page in FIG. 12G and upward and downward dispensing in FIG. 12H) but in a more sprayed manner (similar to FIGS. 12A and 12B) in multiple directions. In FIGS. 12E through 12G, dispensing preventions are shown in a variety of other directions and locations except where dispensing is allowed.

As noted above, FIGS. 12I-12P shows a Configuration B of the reservoir 9100 where the reservoir 9100 includes a main portion 9101 and a delivery arm 9103. Dispensing with respect to the main portion 9101 in these examples can be similar to those discussed above with reference to FIGS. 12A-12H. The main portion 9101 of the implantable medical system 9000 can be similar to those discussed with respect to FIGS. 12A-12H. The examples shown in FIGS. 12I-12P can, in addition or in alternative, include dispensing from the delivery arm 9103 as is further discussed below. While specific examples are discussed below, it should be appreciated that the skilled artisan would recognize many other examples and combinations thereof in light of this disclosure.

Turning to FIGS. 121 and 12J, a multidirectional dispensing operation at the delivery arm 9103 is shown. In this regard, dispensing at the delivery arm 9103 can occur in a variety of directions (e.g., at a tip portion of the delivery arm 9103 and along the length of the delivery arm 9103). Specific to this example, no dispensing occurs at the main portion 9101 of the implantable medical system 9000. In contrast, in the example shown in FIGS. 12M and 12N both the delivery arm 9103 and the main portion 9101 are configured to dispense in multiple directions. For instance, the main portion 9101 can dispense medicament in a similar manner as discussed with respect to FIGS. 12G and 12H, and the delivery arm 9103 can dispense medicament in a similar manner as discussed with respect to FIGS. 121 and 12J.

As previously discussed with reference to FIGS. 12K and 12L, a guided multidirectional dispensing operation at the delivery arm 9103 is shown. In this regard, dispensing at the delivery arm 9103 can occur in a variety of directions at only a tip portion of the delivery arm 9103. Specific to this example, no dispensing occurs at the main portion 9101 of the implantable medical system 9000 or along the length of the delivery arm 9103 separate from the tip portion. In contrast, in the example shown in FIGS. 12O and 12P the delivery arm 9103 is configured for guided multidirectional dispensing, and the main portion 9101 is configured for a focused dispensing. For instance, the main portion 9101 can dispense medicament in a similar manner as discussed with respect to FIGS. 12C and 12D, and the delivery arm 9103 can dispense medicament in a similar manner as discussed with respect to FIGS. 121 and 12J.

Any of the above configurations of the implantable medical system 100 and dispensing mechanisms may be applied for use in treating retinal vein occlusions Under these circumstances, the medicament delivery system (e.g., in Configurations A and C) can be arranged at the posterior location of the eye 5000 and optionally (e.g., in Configurations B and D) at the anterior location of the eye 5000. APIs include monoclonal antibodies such as bevacizmab, ranibizumab, AVASTIN, or LUCENTIS. In examples, APIs include steroids such as dexamethasone or OZURDEX. Drug delivery vehicles can be used during administration of APIs. For example, with respect to steroids, such drug delivery vehicles can include a PLGA matrix with drug, placed in vitreous. API classes can also include small binding molecules defined as antagonist molecules that enable inhibition of angiogenic growth factors, including VEGF or PDGF, or inhibit protein kinases. In further examples, API classes can include antibody mimetic proteins and peptides.

### Keratitis and Dry Eye

The present disclosure includes devices, systems and methods suitable for treatment of corneal diseases. Some such corneal diseased include keratitis and dry eye. In some examples, the reservoir 9100 can dispense medicament from opposing sides of the reservoir 9100 such that the medicament is released in a direction toward and into the eye 5000 as well as in a direction away from the eye 5000 and into the conjunctiva 5002.

### Keratitis

Keratitis can be treated using example implementations of the implantable medical system 9000. Keratitis is inflammation of the cornea. For treatment of keratitis, a medicament delivery system can be subconjunctivally implanted (e.g., near the limbus the of the eye 5000). In examples, the medicament delivery system can be any of Configurations A-D.

For example, the medicament delivery system can be the implantable medical system 9000 as described previously throughout with reference to FIGS. 10A-12P and described again herein. These implantable medical systems 9000 can be similar to others disclosed elsewhere herein, including the medicament delivery systems 1000, 2000, 3000. In this regard, an implantable medical system 9000 can be implanted and arranged at one or more implantation locations or sites to deliver medicament for treatment of keratitis.

As previously described, FIGS. 10 and 10A-10D show several examples of an implantable medical system 9000 that is useful for employing these aspects of the present disclosure. In particular, FIG. 10 is an isometric view of the implantable medical system 9000 having the reservoir 9100 with the main portion 9101 and the delivery arm 9103. FIG. 10A shows the inflated reservoir 9100 with FIGS. 10A-1 and 10A-2 therein showing a microscopic view of a first and second microporous material 9202 respectively in the implantable medical system 9000. FIG. 10B shows the reservoir 9100 in FIG. 10A being refilled via insertion of the syringe 9110 (although other suitable means for refilling the reservoir 9100 in situ may be used). In particular, FIG. 10C shows the inflated reservoir 9100 with the delivery arm 9103. FIGS. 10C-1 and 10C-2 therein showing a microscopic view of a first and second microporous material 9202 respectively in the implantable medical system 9000. FIG. 10D shows the reservoir 9100 in FIG. 10C being refilled via insertion of the syringe 9110 (although other suitable means for refilling the reservoir 9100 in situ may be used). In examples, the main portion 9101 and the delivery arm 9103 can comprise the same or similar materials. Under these circumstances, the delivery arm 9103 can be made integral with or attachable to the main portion 9101 such that the delivery arm 9103 and the main portion 9101 are in fluid communication with each other. The example microporous material is shown as a schematic in FIGS. 10A-1 and 10A-2 for clarity and illustration purposes. The illustrative imagery provided in FIGS. 10A-1, 10A-2, 10C-1, and 10C-2 are representative of the microscopic imagery presented in FIG. 18. Further discussion of microporous materials that are appropriate for employing principles of the present disclosure are shown and described in U.S. Pat. Pub. US2018/0263817, the entire contents of which are hereby incorporated by reference.

The medicant delivery device shown here in FIGS. 10A-10D can be similar to the implantable delivery devices 9000 discussed above. For instance, this device can be used to dispense medicament as discussed above. The implantable medical system 9000 can include the material structure presented in FIGS. 10A-1 and 10C-1 and shown in FIG. 18. The implantable medical system 9000 can also include the material structure, selective permeability, and porous materials shown and described in U.S. Pat. No. 10,849,731 to Cully and as shown and described in U.S. Pat. Publ. No. 2018/0126134 to Cully, both of which are incorporated by reference in their entireties, with particular reference to FIGS. 3-10 and the accompanying descriptions provided in each of those incorporated references. The implantable medical system 9000 can further include the first microporous material 9201 that is bonded to the second microporous material 9202. The first microporous material 9201 can have the first microporous layer 9203 that includes the plurality of pores sized to permit tissue ingrowth. The first microporous material 9201 can have the second microporous layer 9205 that includes the plurality of pores sized to permit tissue ingrowth. The second microporous material 9202 can have the third microporous layer 9205 that includes the plurality of pores sized to resist tissue ingrowth. The second microporous material 9202 can have the fourth microporous layer 9207 that includes the plurality of pores sized to permit tissue ingrowth. The second microporous layer 9205 can be bonded to the third microporous layer 9205 to thereby form the reservoir 9100 for receiving the medicament. The first and second microporous materials 9201 and 9202 can be configured to meter the rate at which the medicament is dispensed from the reservoir 9100 when the delivery device is implanted.

According to principles of the present disclosure, the implantable medical system 9000 can be used to treat keratitis. In general, methods of treating diseases using an implantable medical system 9000 configured to meter a rate at which the medicament is dispensed from the reservoir 9100 when the delivery device is implanted are disclosed. The method can include selecting an implantable medical system 9000 that is similar to the implantable medical system 9000 discussed above. For instance, the first microporous material 9201 can have a first microporous layer 9203 that includes a plurality of pores sized to resist tissue ingrowth. The first microporous material 9201 can have a second microporous layer 9205 that includes a plurality of pores sized to permit tissue ingrowth. The second microporous material 9202 can have a third microporous layer 9205 that includes a plurality of pores sized to resist tissue ingrowth. The second microporous material 9202 can have a fourth microporous layer 9207 that includes a plurality of pores sized to permit tissue ingrowth. The second microporous layer 9205 can be bonded to the third microporous layer 9205 to thereby form a reservoir 9100 for receiving the medicament. The first and second microporous materials 9201 and 9202 can be configured to meter a rate at which the keratitis medicament is dispensed from the reservoir 9100 when the delivery device is implanted.

Administration of the medicament can include several steps. For instance, the method can include filling the reservoir 9100 with a medicament for treating keratitis. In certain instances, portions or all of the first and second microporous material 9202 can be elastomeric such that they reseal after insertion of a syringe 9110 or other similar devices. The method can include implanting the implantable medical system 9000 at an implantation location. The method can include allowing the medicament to dispense from the reservoir 9100 to one or more treatment sites.

FIGS. 11A-11D show various features of the reservoir 9100 in examples of medicament treatment devices. In particular, FIG. 11A shows the first configuration (Configuration “A”) of the reservoir 9100 where the reservoir 9100 includes the main portion 9101 only. FIG. 11B shows the second configuration (Configuration “B”) of the reservoir 9100 where the reservoir 9100 includes the main portion 9101 and the delivery arm 9103. FIG. 11C shows the third configuration (Configuration “C”) of the reservoir 9100 that is similar to that of FIG. 11A and further includes the plurality of chambers 9300 and the plurality of fill ports 9310 with which to fill the corresponding chamber with medicament. FIG. 11D shows the fourth configuration (Configuration “D”) of the reservoir 9100 that is similar to that of FIG. 11B and further includes the plurality of chambers 9300 and the plurality of fill ports 9310 with which to fill the corresponding chamber with medicament. In examples, fill ports 9310 can be an injection site at the reservoir 9100 or a feature of the implantable medical system 9000 that facilitates filling the reservoir 9100 with medicament. As further discussed below, the plurality of chambers 9300 (e.g., in FIGS. 11C and 11D) can be in fluid communication or fluidically isolated from one another. In examples, as further discussed elsewhere herein, the reservoir 9100 can dispense medicament in one direction (e.g., into the page) or in multiple directions (e.g., into the page, out of the page, in-plane with the page, or any combination thereof).

A number of factors can influence physical characteristics of medicament treatment devices. Depending on at least one of the implantation locations and the disease to be treated, one or both of a geometry and functionality of the reservoir 9100 of the medicament delivery system 1000 can vary in examples. For instance, dimensions the reservoir 9100 can be made larger or smaller when compared to other configurations of the medicament delivery system 1000. These variations can depend on, among other things, the amount of medicament to be delivered for treatment, the manner in which the medicament is to be delivered, and the resistance to delivery of medicament presented by an implantation location. As discussed here, in examples, the reservoir 9100 can have a generally uniform shape (e.g., resembling an ellipse, a circle, a polygon, etc.). In other examples, the reservoir 9100 can have an irregular shape (e.g., a generally uniform shape with one or more extrusions or protrusions therefrom, an eccentric shape, etc.). Some example shapes of the reservoir 9100 are discussed below with respect to certain example implementations of the medicament delivery system 1000. In examples, when the reservoir 9100 is inflated, the reservoir 9100 can bulge about along a length of the reservoir 9100. In some instances, bulging may cause the reservoir 9100 to balloon or to inflate to a generally polyhedron shape.

In certain examples, the physical characteristics of the reservoir 9100 can facilitate functionality of the medicament delivery system 1000. For instance, the reservoir 9100 can be formed to deliver medicament to a pinpoint location relative to the reservoir 9100, generally disperse medicament over a treatment site at one or more sides of the medicament delivery system 1000, and the like. In this regard, it can be said that the implantable medical system 9000 can have one-directional dispensing (e.g., at one side of the implantable medical system 9000) or multidirectional dispensing (e.g., at multiple sides of the implantable medical system 9000). In examples, one or more locations at the reservoir 9100 can function as the fill port 9310 with which to fill the reservoir 9100 with medicament. In this regard, the implantable medical system 9000 can be refillable with medicament as further discussed above. For example, the main portion 9101 of the reservoir 9100 can function as a refillable chamber, and the delivery arm 9103 can function as a conduit with which to deliver fluid from the refillable chamber. After implantation, medicament can be flushed or drained from the fill port 9310 at the reservoir 9100 to a treatment location. In examples, the reservoir 9100 can include the plurality of chambers 9300 therein with which to store medicament to be delivered. Under these circumstances, one chamber in the plurality of chambers 9300 can include a first medicament, and another chamber in the plurality of chambers 9300 can include a second medicament that is the same or different from the first medicament. Chambers 9300 in the plurality of chambers 9300 can be in fluid communication with one another or isolated from one another depending on the implementation.

Formed at the periphery of the reservoir 9100 can be the extended delivery arm 9103 through which medicament from the reservoir 9100 can be administered. In this regard, the reservoir 9100 can include the reservoir main portion 9101 and the extended delivery arm 9103 extending from the reservoir main portion 9101. For instance, the extended delivery arm 9103 can include a port through which medicament is exclusively allowed to exit the reservoir 9100. In other instances, medicament can be allowed exit the reservoir 9100 at any portion (e.g., not exclusively at the port) of the extended delivery arm 9103. In addition, or in alternative, medicament can be allowed to exit the reservoir 9100 both at the reservoir main portion 9101 and the extended delivery arm 9103.

FIGS. 12A-12P show various dispensing configurations of the reservoir 9100. In particular, FIGS. 12A-12H show the implantable medical system 9000 dispensing when in Configuration A of the reservoir 9100 where the reservoir 9100 includes the main portion 9101 only. FIGS. 12I-12P shows a Configuration B of the reservoir 9100 where the reservoir 9100 includes the main portion 9101 and the delivery arm 9103. Although for conciseness, the dispensing operations here are shown with respect to Configurations A and B, one skilled in the art will appreciate that these dispensing operations are similarly applicable to Configurations C and D. In this regard, the distinction would be that Configurations C and D include multiple chambers that may be similarly dispensed to the single chambers discussed below with respect to Configurations A and B. For illustration purposes, dispensing directions are shown as straight arrows flowing out of the reservoir 9100, and dispensing preventions are shown as curly arrows at the interior of the reservoir 9100. Dispensing can occur at only portions of the implantable medical system 9000 that are permeable, and dispensing preventions can occur at impermeable portions of the implantable medical system 9000. In addition, flow and directions into the page are shown as an encircled X, and directions out of the page are shown as encircled dots. Of course, these are merely schematic representations of a more complex phenomena but are used here for clarity of discussion.

As will be discussed below in further detail, dispensing of the medicament can vary across configurations and within configurations when in use for treating keratitis. For instance, as discussed above, the implantable medical system 9000 can be configured such that medicament is generally dispensed in one direction or in a plurality of directions. Under these circumstances, dispensing can occur at the main portion 9101, the delivery arm 9103, or both the main portion 9101 and the delivery arm 9103 of the implantable medical system 9000. In further under these circumstances, directionality of dispensing at the main portion 9101, the delivery arm 9103, or both the main portion 9101 and delivery arm 9103 of the implantable medical system 9000 can be the same or can be different depending on the example. The example shown and discussed in these figures are just some of many examples disclosed herein. One skilled in the art will appreciate this fact and recognize that, in reading this disclosure as a whole, certain variations and modifications of these examples are logical extensions of the examples discussed below.

With reference again to FIGS. 12A and 12B, a single direction dispensing is shown (generally into the page in FIG. 12A and downward in FIG. 12B). Distribution of dispensing is shown in a sprayed pattern where the directionality of the arrows generally deviates from one another but in the same general direction. In contrast, FIGS. 12C and 12D show a similar dispensing operation (generally into the page in FIG. 12B and downward dispensing in FIG. 12D) but in a more focused manner where the directionality of the arrows generally do not deviate from one another and are in the same direction. In FIGS. 12A through 12D, dispensing preventions are shown in a variety of other directions and locations except where dispensing is allowed.

Turning again to FIGS. 12E and 12F, a multidirectional dispensing operation is shown (e.g., generally into and out of the page in FIG. 12E and upward and downward in FIG. 12F). Distribution of dispensing is shown in a focused manner (similar to FIGS. 12C and 12D) in multiple directions. As is indicated by the number of arrows, dispensing out of the page and FIG. 12E and upward in FIG. 12F is less than dispensing into the page in FIG. 12E and downward in FIG. 12F. Although shown in the same focused manner of dispensing in multiple directions, it should be appreciated that some examples include dispensing in the sprayed manner in one direction and a focused manner in another direction. It should also be appreciated that dispensing may be the same amount in each direction of dispensing or may be different amounts in each direction of dispensing. As discussed above, an amount of dispensing can be proportional to permeability at the dispensing portion of the implantable medical system 9000. As previously described, FIGS. 12G and 12H show a similar dispensing operation (generally into and out of the page in FIG. 12G and upward and downward dispensing in FIG. 12H) but in a more sprayed manner (similar to FIGS. 12A and 12B) in multiple directions. In FIGS. 12E through 12G, dispensing preventions are shown in a variety of other directions and locations except where dispensing is allowed.

As noted above, FIGS. 12I-12P shows a Configuration B of the reservoir 9100 where the reservoir 9100 includes a main portion 9101 and a delivery arm 9103. Dispensing with respect to the main portion 9101 in these examples can be similar to those discussed above with reference to FIGS. 12A-12H. The main portion 9101 of the implantable medical system 9000 can be similar to those discussed with respect to FIGS. 12A-12H. The examples shown in FIGS. 12I-12P can, in addition or in alternative, include dispensing from the delivery arm 9103 as is further discussed below. While specific examples are discussed below, it should be appreciated that the skilled artisan would recognize many other examples and combinations thereof in light of this disclosure.

Turning to FIGS. 12I and 12J, a multidirectional dispensing operation at the delivery arm 9103 is shown. In this regard, dispensing at the delivery arm 9103 can occur in a variety of directions (e.g., at a tip portion of the delivery arm 9103 and along the length of the delivery arm 9103). Specific to this example, no dispensing occurs at the main portion 9101 of the implantable medical system 9000. In contrast, in the example shown in FIGS. 12M and 12N both the delivery arm 9103 and the main portion 9101 are configured to dispense in multiple directions. For instance, the main portion 9101 can dispense medicament in a similar manner as discussed with respect to FIGS. 12G and 12H, and the delivery arm 9103 can dispense medicament in a similar manner as discussed with respect to FIGS. 121 and 12J.

As previously discussed with reference to FIGS. 12K and 12L, a guided multidirectional dispensing operation at the delivery arm 9103 is shown. In this regard, dispensing at the delivery arm 9103 can occur in a variety of directions at only a tip portion of the delivery arm 9103. Specific to this example, no dispensing occurs at the main portion 9101 of the implantable medical system 9000 or along the length of the delivery arm 9103 separate from the tip portion. In contrast, in the example shown in FIGS. 12O and 12P the delivery arm 9103 is configured for guided multidirectional dispensing, and the main portion 9101 is configured for a focused dispensing. For instance, the main portion 9101 can dispense medicament in a similar manner as discussed with respect to FIGS. 12C and 12D, and the delivery arm 9103 can dispense medicament in a similar manner as discussed with respect to FIGS. 121 and 12J. Any of the above configurations of the implantable medical system 100 and dispensing mechanisms may be applied for use in treating keratitis.

Under these circumstances, the medicament delivery system (e.g., in Configurations A and C) can be arranged at the anterior location of the eye 5000 and optionally (e.g., in Configurations B and D) in the anterior chamber 5006 of the eye 5000. Example APIs include antibiotics, steroids, or antifungal agents.

### Dry Eye

Dry eye is another corneal disease that can be treated using example implementations of the implantable medical system 9000. Dry eye is inadequate tear film lubrication of the eye 5000. For treatment of dry eye, a medicament delivery system can be subconjunctivally implanted. In examples, the medicament delivery system can be any of Configurations A-D. For example, the medicament delivery system can be the implantable medical system 9000 as described previously throughout with reference to FIGS. 10A-12P and described again herein. These implantable medical systems 9000 can be similar to others disclosed elsewhere herein, including the medicament delivery systems 1000, 2000, 3000. In this regard, an implantable medical system 9000 can be implanted and arranged at one or more implantation locations or sites to deliver medicament for treatment of dry eye.

As previously described, FIGS. 10 and 10A-10D show several examples of an implantable medical system 9000 that is useful for employing these aspects of the present disclosure. In particular, FIG. 10 is an isometric view of the implantable medical system 9000 having the reservoir 9100 with the main portion 9101 and the delivery arm 9103. FIG. 10A shows the inflated reservoir 9100 with FIGS. 10A-1 and 10A-2 therein showing a microscopic view of a first and second microporous material 9202 respectively in the implantable medical system 9000. FIG. 10B shows the reservoir 9100 in FIG. 10A being refilled via insertion of the syringe 9110 (although other suitable means for refilling the reservoir 9100 in situ may be used). In particular, FIG. 10C shows the inflated reservoir 9100 with the delivery arm 9103. FIGS. 10C-1 and 10C-2 therein showing a microscopic view of a first and second microporous material 9202 respectively in the implantable medical system 9000. FIG. 10D shows the reservoir 9100 in FIG. 10C being refilled via insertion of the syringe 9110 (although other suitable means for refilling the reservoir 9100 in situ may be used). In examples, the main portion 9101 and the delivery arm 9103 can comprise the same or similar materials. Under these circumstances, the delivery arm 9103 can be made integral with or attachable to the main portion 9101 such that the delivery arm 9103 and the main portion 9101 are in fluid communication with each other. The example microporous material is shown as a schematic in FIGS. 10A-1 and 10A-2 for clarity and illustration purposes. The illustrative imagery provided in FIGS. 10A-1, 10A-2, 10C-1, and 10C-2 are representative of the microscopic imagery presented in FIG. 18. Further discussion of microporous materials that are appropriate for employing principles of the present disclosure are shown and described in U.S. Pat. Pub. US2018/0263817, the entire contents of which are hereby incorporated by reference.

The medicant delivery device shown here in FIGS. 10A-10D can be similar to the implantable delivery devices 9000 discussed above. For instance, this device can be used to dispense medicament as discussed above. The implantable medical system 9000 can include the material structure presented in FIGS. 10A-1 and 10C-1 and shown in FIG. 18. The implantable medical system 9000 can also include the material structure, selective permeability, and porous materials shown and described in U.S. Pat. No. 10,849,731 to Cully and as shown and described in U.S. Pat. Publ. No. 2018/0126134 to Cully, both of which are incorporated by reference in their entireties, with particular reference to FIGS. 3-10 and the accompanying descriptions provided in each of those incorporated references. The implantable medical system 9000 can further include the first microporous material 9201 that is bonded to the second microporous material 9202. The first microporous material 9201 can have the first microporous layer 9203 that includes the plurality of pores sized to permit tissue ingrowth. The first microporous material 9201 can have the second microporous layer 9205 that includes the plurality of pores sized to permit tissue ingrowth. The second microporous material 9202 can have the third microporous layer 9205 that includes the plurality of pores sized to resist tissue ingrowth. The second microporous material 9202 can have the fourth microporous layer 9207 that includes the plurality of pores sized to permit tissue ingrowth. The second microporous layer 9205 can be bonded to the third microporous layer 9205 to thereby form the reservoir 9100 for receiving the medicament. The first and second microporous materials 9201 and 9202 can be configured to meter the rate at which the medicament is dispensed from the reservoir 9100 when the delivery device is implanted.

According to principles of the present disclosure, the implantable medical system 9000 can be used to treat dry eye. In general, methods of treating diseases using an implantable medical system 9000 configured to meter a rate at which the medicament is dispensed from the reservoir 9100 when the delivery device is implanted are disclosed. The method can include selecting an implantable medical system 9000 that is similar to the implantable medical system 9000 discussed above. For instance, the first microporous material 9201 can have a first microporous layer 9203 that includes a plurality of pores sized to resist tissue ingrowth. The first microporous material 9201 can have a second microporous layer 9205 that includes a plurality of pores sized to permit tissue ingrowth. The second microporous material 9202 can have a third microporous layer 9205 that includes a plurality of pores sized to resist tissue ingrowth. The second microporous material 9202 can have a fourth microporous layer 9207 that includes a plurality of pores sized to permit tissue ingrowth. The second microporous layer 9205 can be bonded to the third microporous layer 9205 to thereby form a reservoir 9100 for receiving the medicament. The first and second microporous materials 9201 and 9202 can be configured to meter a rate at which the dry eye medicament is dispensed from the reservoir 9100 when the delivery device is implanted.

Administration of the medicament can include several steps. For instance, the method can include filling the reservoir 9100 with a medicament for treating dry eye. In certain instances, portions or all of the first and second microporous material 9202 can be elastomeric such that they reseal after insertion of a syringe 9110 or other similar devices. The method can include implanting the implantable medical system 9000 at an implantation location. The method can include allowing the medicament to dispense from the reservoir 9100 to one or more treatment sites.

FIGS. 11A-11D show various features of the reservoir 9100 in examples of medicament treatment devices. In particular, FIG. 11A shows the first configuration (Configuration “A”) of the reservoir 9100 where the reservoir 9100 includes the main portion 9101 only. FIG. 11B shows the second configuration (Configuration “B”) of the reservoir 9100 where the reservoir 9100 includes the main portion 9101 and the delivery arm 9103. FIG. 11C shows the third configuration (Configuration “C”) of the reservoir 9100 that is similar to that of FIG. 11A and further includes the plurality of chambers 9300 and the plurality of fill ports 9310 with which to fill the corresponding chamber with medicament. FIG. 11D shows the fourth configuration (Configuration “D”) of the reservoir 9100 that is similar to that of FIG. 11B and further includes the plurality of chambers 9300 and the plurality of fill ports 9310 with which to fill the corresponding chamber with medicament. In examples, fill ports 9310 can be an injection site at the reservoir 9100 or a feature of the implantable medical system 9000 that facilitates filling the reservoir 9100 with medicament. As further discussed below, the plurality of chambers 9300 (e.g., in FIGS. 11C and 11D) can be in fluid communication or fluidically isolated from one another. In examples, as further discussed elsewhere herein, the reservoir 9100 can dispense medicament in one direction (e.g., into the page) or in multiple directions (e.g., into the page, out of the page, in-plane with the page, or any combination thereof).

A number of factors can influence physical characteristics of medicament treatment devices. Depending on at least one of the implantation locations and the disease to be treated, one or both of a geometry and functionality of the reservoir 9100 of the medicament delivery system 1000 can vary in examples. For instance, dimensions the reservoir 9100 can be made larger or smaller when compared to other configurations of the medicament delivery system 1000. These variations can depend on, among other things, the amount of medicament to be delivered for treatment, the manner in which the medicament is to be delivered, and the resistance to delivery of medicament presented by an implantation location. As discussed here, in examples, the reservoir 9100 can have a generally uniform shape (e.g., resembling an ellipse, a circle, a polygon, etc.). In other examples, the reservoir 9100 can have an irregular shape (e.g., a generally uniform shape with one or more extrusions or protrusions therefrom, an eccentric shape, etc.). Some example shapes of the reservoir 9100 are discussed below with respect to certain example implementations of the medicament delivery system 1000. In examples, when the reservoir 9100 is inflated, the reservoir 9100 can bulge about along a length of the reservoir 9100. In some instances, bulging may cause the reservoir 9100 to balloon or to inflate to a generally polyhedron shape.

In certain examples, the physical characteristics of the reservoir 9100 can facilitate functionality of the medicament delivery system 1000. For instance, the reservoir 9100 can be formed to deliver medicament to a pinpoint location relative to the reservoir 9100, generally disperse medicament over a treatment site at one or more sides of the medicament delivery system 1000, and the like. In this regard, it can be said that the implantable medical system 9000 can have one-directional dispensing (e.g., at one side of the implantable medical system 9000) or multidirectional dispensing (e.g., at multiple sides of the implantable medical system 9000). In examples, one or more locations at the reservoir 9100 can function as the fill port 9310 with which to fill the reservoir 9100 with medicament. In this regard, the implantable medical system 9000 can be refillable with medicament as further discussed above. For example, the main portion 9101 of the reservoir 9100 can function as a refillable chamber, and the delivery arm 9103 can function as a conduit with which to deliver fluid from the refillable chamber. After implantation, medicament can be flushed or drained from the fill port 9310 at the reservoir 9100 to a treatment location. In examples, the reservoir 9100 can include the plurality of chambers 9300 therein with which to store medicament to be delivered. Under these circumstances, one chamber in the plurality of chambers 9300 can include a first medicament, and another chamber in the plurality of chambers 9300 can include a second medicament that is the same or different from the first medicament. Chambers 9300 in the plurality of chambers 9300 can be in fluid communication with one another or isolated from one another depending on the implementation.

Formed at the periphery of the reservoir 9100 can be the extended delivery arm 9103 through which medicament from the reservoir 9100 can be administered. In this regard, the reservoir 9100 can include the reservoir main portion 9101 and the extended delivery arm 9103 extending from the reservoir main portion 9101. For instance, the extended delivery arm 9103 can include a port through which medicament is exclusively allowed to exit the reservoir 9100. In other instances, medicament can be allowed exit the reservoir 9100 at any portion (e.g., not exclusively at the port) of the extended delivery arm 9103. In addition, or in alternative, medicament can be allowed to exit the reservoir 9100 both at the reservoir main portion 9101 and the extended delivery arm 9103.

FIGS. 12A-12P show various dispensing configurations of the reservoir 9100. In particular, FIGS. 12A-12H show the implantable medical system 9000 dispensing when in Configuration A of the reservoir 9100 where the reservoir 9100 includes the main portion 9101 only. FIGS. 12I-12P shows a Configuration B of the reservoir 9100 where the reservoir 9100 includes the main portion 9101 and the delivery arm 9103. Although for conciseness, the dispensing operations here are shown with respect to Configurations A and B, one skilled in the art will appreciate that these dispensing operations are similarly applicable to Configurations C and D. In this regard, the distinction would be that Configurations C and D include multiple chambers that may be similarly dispensed to the single chambers discussed below with respect to Configurations A and B. For illustration purposes, dispensing directions are shown as straight arrows flowing out of the reservoir 9100, and dispensing preventions are shown as curly arrows at the interior of the reservoir 9100. Dispensing can occur at only portions of the implantable medical system 9000 that are permeable, and dispensing preventions can occur at impermeable portions of the implantable medical system 9000. In addition, flow and directions into the page are shown as an encircled X, and directions out of the page are shown as encircled dots. Of course, these are merely schematic representations of a more complex phenomena but are used here for clarity of discussion.

As will be discussed below in further detail, dispensing of the medicament can vary across configurations and within configurations. For instance, as discussed above, the implantable medical system 9000 can be configured such that medicament is generally dispensed in one direction or in a plurality of directions. Under these circumstances, dispensing can occur at the main portion 9101, the delivery arm 9103, or both the main portion 9101 and the delivery arm 9103 of the implantable medical system 9000. In further under these circumstances, directionality of dispensing at the main portion 9101, the delivery arm 9103, or both the main portion 9101 and delivery arm 9103 of the implantable medical system 9000 can be the same or can be different depending on the example. The example shown and discussed in these figures are just some of many examples disclosed herein. One skilled in the art will appreciate this fact and recognize that, in reading this disclosure as a whole, certain variations and modifications of these examples are logical extensions of the examples discussed below.

With reference again to FIGS. 12A and 12B, a single direction dispensing is shown (generally into the page in FIG. 12A and downward in FIG. 12B). Distribution of dispensing is shown in a sprayed pattern where the directionality of the arrows generally deviates from one another but in the same general direction. In contrast, FIGS. 12C and 12D show a similar dispensing operation (generally into the page in FIG. 12B and downward dispensing in FIG. 12D) but in a more focused manner where the directionality of the arrows generally do not deviate from one another and are in the same direction. In FIGS. 12A through 12D, dispensing preventions are shown in a variety of other directions and locations except where dispensing is allowed.

Turning again to FIGS. 12E and 12F, a multidirectional dispensing operation is shown (e.g., generally into and out of the page in FIG. 12E and upward and downward in FIG. 12F). Distribution of dispensing is shown in a focused manner (similar to FIGS. 12C and 12D) in multiple directions. As is indicated by the number of arrows, dispensing out of the page and FIG. 12E and upward in FIG. 12F is less than dispensing into the page in FIG. 12E and downward in FIG. 12F. Although shown in the same focused manner of dispensing in multiple directions, it should be appreciated that some examples include dispensing in the sprayed manner in one direction and a focused manner in another direction. It should also be appreciated that dispensing may be the same amount in each direction of dispensing or may be different amounts in each direction of dispensing. As discussed above, an amount of dispensing can be proportional to permeability at the dispensing portion of the implantable medical system 9000. As previously described, FIGS. 12G and 12H show a similar dispensing operation (generally into and out of the page in FIG. 12G and upward and downward dispensing in FIG. 12H) but in a more sprayed manner (similar to FIGS. 12A and 12B) in multiple directions. In FIGS. 12E through 12G, dispensing preventions are shown in a variety of other directions and locations except where dispensing is allowed.

As noted above, FIGS. 12I-12P shows a Configuration B of the reservoir 9100 where the reservoir 9100 includes a main portion 9101 and a delivery arm 9103. Dispensing with respect to the main portion 9101 in these examples can be similar to those discussed above with reference to FIGS. 12A-12H. The main portion 9101 of the implantable medical system 9000 can be similar to those discussed with respect to FIGS. 12A-12H. The examples shown in FIGS. 12I-12P can, in addition or in alternative, include dispensing from the delivery arm 9103 as is further discussed below. While specific examples are discussed below, it should be appreciated that the skilled artisan would recognize many other examples and combinations thereof in light of this disclosure.

Turning to FIGS. 121 and 12J, a multidirectional dispensing operation at the delivery arm 9103 is shown. In this regard, dispensing at the delivery arm 9103 can occur in a variety of directions (e.g., at a tip portion of the delivery arm 9103 and along the length of the delivery arm 9103). Specific to this example, no dispensing occurs at the main portion 9101 of the implantable medical system 9000. In contrast, in the example shown in FIGS. 12M and 12N both the delivery arm 9103 and the main portion 9101 are configured to dispense in multiple directions. For instance, the main portion 9101 can dispense medicament in a similar manner as discussed with respect to FIGS. 12G and 12H, and the delivery arm 9103 can dispense medicament in a similar manner as discussed with respect to FIGS. 121 and 12J.

As previously discussed with reference to FIGS. 12K and 12L, a guided multidirectional dispensing operation at the delivery arm 9103 is shown. In this regard, dispensing at the delivery arm 9103 can occur in a variety of directions at only a tip portion of the delivery arm 9103. Specific to this example, no dispensing occurs at the main portion 9101 of the implantable medical system 9000 or along the length of the delivery arm 9103 separate from the tip portion. In contrast, in the example shown in FIGS. 12O and 12P the delivery arm 9103 is configured for guided multidirectional dispensing, and the main portion 9101 is configured for a focused dispensing. For instance, the main portion 9101 can dispense medicament in a similar manner as discussed with respect to FIGS. 12C and 12D, and the delivery arm 9103 can dispense medicament in a similar manner as discussed with respect to FIGS. 121 and 12J.

Any of the above configurations of the implantable medical system 100 and dispensing mechanisms may be applied for use in treating dry eye. Under these circumstances, the medicament delivery system (e.g., in Configurations A and C) can be arranged at the anterior location of the eye 5000 and optionally (e.g., in Configurations B and D) in the anterior chamber 5006 of the eye 5000. Example APIs include cyclosporine, lifitegrast, RESTASIS, or XIIDRA.

### Uveitis

Uveitis is yet another disease that can be treated using example implementations of the implantable medical system 9000. Uveitis is a disease of the eye 5000 that involves inflammation of the uvea. For treatment of uveitis, a medicament delivery system can be suprachoroidally implanted (e.g., posterior of the pars plana of the eye 5000). In examples, the medicament delivery system can be any of Configurations A-D. Under these circumstances, the medicament delivery system (e.g., in Configurations A and C) can be arranged at the posterior location of the eye 5000 and optionally (e.g., in Configurations B and D) at the anterior location of the eye 5000. In some such circumstances, the medicament delivery system (e.g., in Configurations B and D) is positioned at the anterior location of the eye 5000 and arranged for intravitreal administration. API classes can include corticosteroid such as fluocinolone acetonide and RETISERT. Drug delivery vehicles can be used during administration of APIs. For example, with respect to RETISERT, such drug delivery vehicles can include a silicone cup with PVA membrane, which can deliver the medicament over about 2-3 years.

### Presbyopia

Still yet another disease that can be treated using example implementations of the implantable medical system 9000 is presbyopia, which is a crystalline stiffening of the lens of the eye 5000. For treatment of presbyopia, a medicament delivery system can be subconjunctivally implanted. In examples, the medicament delivery system can be any of Configurations A-D. For example, the medicament delivery system can be the implantable medical system 9000 as described previously throughout with reference to FIGS. 10A-12P and described again herein. These implantable medical systems 9000 can be similar to others disclosed elsewhere herein, including the medicament delivery systems 1000, 2000, 3000. In this regard, an implantable medical system 9000 can be implanted and arranged at one or more implantation locations or sites to deliver medicament for treatment of presbyopia.

As previously described, FIGS. 10 and 10A-10D show several examples of an implantable medical system 9000 that is useful for employing these aspects of the present disclosure. In particular, FIG. 10 is an isometric view of the implantable medical system 9000 having the reservoir 9100 with the main portion 9101 and the delivery arm 9103. FIG. 10A shows the inflated reservoir 9100 with FIGS. 10A-1 and 10A-2 therein showing a microscopic view of a first and second microporous material 9202 respectively in the implantable medical system 9000. FIG. 10B shows the reservoir 9100 in FIG. 10A being refilled via insertion of the syringe 9110 (although other suitable means for refilling the reservoir 9100 in situ may be used). In particular, FIG. 10C shows the inflated reservoir 9100 with the delivery arm 9103. FIGS. 10C-1 and 10C-2 therein showing a microscopic view of a first and second microporous material 9202 respectively in the implantable medical system 9000. FIG. 10D shows the reservoir 9100 in FIG. 10C being refilled via insertion of the syringe 9110 (although other suitable means for refilling the reservoir 9100 in situ may be used). In examples, the main portion 9101 and the delivery arm 9103 can comprise the same or similar materials. Under these circumstances, the delivery arm 9103 can be made integral with or attachable to the main portion 9101 such that the delivery arm 9103 and the main portion 9101 are in fluid communication with each other. The example microporous material is shown as a schematic in FIGS. 10A-1 and 10A-2 for clarity and illustration purposes. The illustrative imagery provided in FIGS. 10A-1, 10A-2, 10C-1, and 10C-2 are representative of the microscopic imagery presented in FIG. 18. Further discussion of microporous materials that are appropriate for employing principles of the present disclosure are shown and described in U.S. Pat. Pub. US2018/0263817, the entire contents of which are hereby incorporated by reference.

The medicant delivery device shown here in FIGS. 10A-10D can be similar to the implantable delivery devices 9000 discussed above. For instance, this device can be used to dispense medicament as discussed above. The implantable medical system 9000 can include the material structure presented in FIGS. 10A-1 and 10C-1 and shown in FIG. 18. The implantable medical system 9000 can also include the material structure, selective permeability, and porous materials shown and described in U.S. Pat. No. 10,849,731 to Cully and as shown and described in U.S. Pat. Publ. No. 2018/0126134 to Cully, both of which are incorporated by reference in their entireties, with particular reference to FIGS. 3-10 and the accompanying descriptions provided in each of those incorporated references. The implantable medical system 9000 can further include the first microporous material 9201 that is bonded to the second microporous material 9202. The first microporous material 9201 can have the first microporous layer 9203 that includes the plurality of pores sized to permit tissue ingrowth. The first microporous material 9201 can have the second microporous layer 9205 that includes the plurality of pores sized to permit tissue ingrowth. The second microporous material 9202 can have the third microporous layer 9205 that includes the plurality of pores sized to resist tissue ingrowth. The second microporous material 9202 can have the fourth microporous layer 9207 that includes the plurality of pores sized to permit tissue ingrowth. The second microporous layer 9205 can be bonded to the third microporous layer 9205 to thereby form the reservoir 9100 for receiving the medicament. The first and second microporous materials 9201 and 9202 can be configured to meter the rate at which the medicament is dispensed from the reservoir 9100 when the delivery device is implanted.

According to principles of the present disclosure, the implantable medical system 9000 can be used to treat presbyopia. In general, methods of treating diseases using an implantable medical system 9000 configured to meter a rate at which the medicament is dispensed from the reservoir 9100 when the delivery device is implanted are disclosed. The method can include selecting an implantable medical system 9000 that is similar to the implantable medical system 9000 discussed above. For instance, the first microporous material 9201 can have a first microporous layer 9203 that includes a plurality of pores sized to resist tissue ingrowth. The first microporous material 9201 can have a second microporous layer 9205 that includes a plurality of pores sized to permit tissue ingrowth. The second microporous material 9202 can have a third microporous layer 9205 that includes a plurality of pores sized to resist tissue ingrowth. The second microporous material 9202 can have a fourth microporous layer 9207 that includes a plurality of pores sized to permit tissue ingrowth. The second microporous layer 9205 can be bonded to the third microporous layer 9205 to thereby form a reservoir 9100 for receiving the medicament. The first and second microporous materials 9201 and 9202 can be configured to meter a rate at which the presbyopia medicament is dispensed from the reservoir 9100 when the delivery device is implanted.

Administration of the medicament can include several steps. For instance, the method can include filling the reservoir 9100 with a medicament for treating presbyopia. In certain instances, portions or all of the first and second microporous material 9202 can be elastomeric such that they reseal after insertion of a syringe 9110 or other similar devices. The method can include implanting the implantable medical system 9000 at an implantation location. The method can include allowing the medicament to dispense from the reservoir 9100 to one or more treatment sites.

FIGS. 11A-11D show various features of the reservoir 9100 in examples of medicament treatment devices. In particular, FIG. 11A shows the first configuration (Configuration “A”) of the reservoir 9100 where the reservoir 9100 includes the main portion 9101 only. FIG. 11B shows the second configuration (Configuration “B”) of the reservoir 9100 where the reservoir 9100 includes the main portion 9101 and the delivery arm 9103. FIG. 11C shows the third configuration (Configuration “C”) of the reservoir 9100 that is similar to that of FIG. 11A and further includes the plurality of chambers 9300 and the plurality of fill ports 9310 with which to fill the corresponding chamber with medicament. FIG. 11D shows the fourth configuration (Configuration “D”) of the reservoir 9100 that is similar to that of FIG. 11B and further includes the plurality of chambers 9300 and the plurality of fill ports 9310 with which to fill the corresponding chamber with medicament. In examples, fill ports 9310 can be an injection site at the reservoir 9100 or a feature of the implantable medical system 9000 that facilitates filling the reservoir 9100 with medicament. As further discussed below, the plurality of chambers 9300 (e.g., in FIGS. 11C and 11D) can be in fluid communication or fluidically isolated from one another. In examples, as further discussed elsewhere herein, the reservoir 9100 can dispense medicament in one direction (e.g., into the page) or in multiple directions (e.g., into the page, out of the page, in-plane with the page, or any combination thereof).

A number of factors can influence physical characteristics of medicament treatment devices. Depending on at least one of the implantation locations and the disease to be treated, one or both of a geometry and functionality of the reservoir 9100 of the medicament delivery system 1000 can vary in examples. For instance, dimensions the reservoir 9100 can be made larger or smaller when compared to other configurations of the medicament delivery system 1000. These variations can depend on, among other things, the amount of medicament to be delivered for treatment, the manner in which the medicament is to be delivered, and the resistance to delivery of medicament presented by an implantation location. As discussed here, in examples, the reservoir 9100 can have a generally uniform shape (e.g., resembling an ellipse, a circle, a polygon, etc.). In other examples, the reservoir 9100 can have an irregular shape (e.g., a generally uniform shape with one or more extrusions or protrusions therefrom, an eccentric shape, etc.). Some example shapes of the reservoir 9100 are discussed below with respect to certain example implementations of the medicament delivery system 1000. In examples, when the reservoir 9100 is inflated, the reservoir 9100 can bulge about along a length of the reservoir 9100. In some instances, bulging may cause the reservoir 9100 to balloon or to inflate to a generally polyhedron shape.

In certain examples, the physical characteristics of the reservoir 9100 can facilitate functionality of the medicament delivery system 1000. For instance, the reservoir 9100 can be formed to deliver medicament to a pinpoint location relative to the reservoir 9100, generally disperse medicament over a treatment site at one or more sides of the medicament delivery system 1000, and the like. In this regard, it can be said that the implantable medical system 9000 can have one-directional dispensing (e.g., at one side of the implantable medical system 9000) or multidirectional dispensing (e.g., at multiple sides of the implantable medical system 9000). In examples, one or more locations at the reservoir 9100 can function as the fill port 9310 with which to fill the reservoir 9100 with medicament. In this regard, the implantable medical system 9000 can be refillable with medicament as further discussed above. For example, the main portion 9101 of the reservoir 9100 can function as a refillable chamber, and the delivery arm 9103 can function as a conduit with which to deliver fluid from the refillable chamber. After implantation, medicament can be flushed or drained from the fill port 9310 at the reservoir 9100 to a treatment location. In examples, the reservoir 9100 can include the plurality of chambers 9300 therein with which to store medicament to be delivered. Under these circumstances, one chamber in the plurality of chambers 9300 can include a first medicament, and another chamber in the plurality of chambers 9300 can include a second medicament that is the same or different from the first medicament. Chambers 9300 in the plurality of chambers 9300 can be in fluid communication with one another or isolated from one another depending on the implementation.

Formed at the periphery of the reservoir 9100 can be the extended delivery arm 9103 through which medicament from the reservoir 9100 can be administered. In this regard, the reservoir 9100 can include the reservoir main portion 9101 and the extended delivery arm 9103 extending from the reservoir main portion 9101. For instance, the extended delivery arm 9103 can include a port through which medicament is exclusively allowed to exit the reservoir 9100. In other instances, medicament can be allowed exit the reservoir 9100 at any portion (e.g., not exclusively at the port) of the extended delivery arm 9103. In addition, or in alternative, medicament can be allowed to exit the reservoir 9100 both at the reservoir main portion 9101 and the extended delivery arm 9103.

FIGS. 12A-12P show various dispensing configurations of the reservoir 9100. In particular, FIGS. 12A-12H show the implantable medical system 9000 dispensing when in Configuration A of the reservoir 9100 where the reservoir 9100 includes the main portion 9101 only. FIGS. 12I-12P shows a Configuration B of the reservoir 9100 where the reservoir 9100 includes the main portion 9101 and the delivery arm 9103. Although for conciseness, the dispensing operations here are shown with respect to Configurations A and B, one skilled in the art will appreciate that these dispensing operations are similarly applicable to Configurations C and D. In this regard, the distinction would be that Configurations C and D include multiple chambers that may be similarly dispensed to the single chambers discussed below with respect to Configurations A and B. For illustration purposes, dispensing directions are shown as straight arrows flowing out of the reservoir 9100, and dispensing preventions are shown as curly arrows at the interior of the reservoir 9100. Dispensing can occur at only portions of the implantable medical system 9000 that are permeable, and dispensing preventions can occur at impermeable portions of the implantable medical system 9000. In addition, flow and directions into the page are shown as an encircled X, and directions out of the page are shown as encircled dots. Of course, these are merely schematic representations of a more complex phenomena but are used here for clarity of discussion.

As will be discussed below in further detail, dispensing of the medicament can vary across configurations and within configurations. For instance, as discussed above, the implantable medical system 9000 can be configured such that medicament is generally dispensed in one direction or in a plurality of directions. Under these circumstances, dispensing can occur at the main portion 9101, the delivery arm 9103, or both the main portion 9101 and the delivery arm 9103 of the implantable medical system 9000. In further under these circumstances, directionality of dispensing at the main portion 9101, the delivery arm 9103, or both the main portion 9101 and delivery arm 9103 of the implantable medical system 9000 can be the same or can be different depending on the example. The example shown and discussed in these figures are just some of many examples disclosed herein. One skilled in the art will appreciate this fact and recognize that, in reading this disclosure as a whole, certain variations and modifications of these examples are logical extensions of the examples discussed below.

With reference again to FIGS. 12A and 12B, a single direction dispensing is shown (generally into the page in FIG. 12A and downward in FIG. 12B). Distribution of dispensing is shown in a sprayed pattern where the directionality of the arrows generally deviates from one another but in the same general direction. In contrast, FIGS. 12C and 12D show a similar dispensing operation (generally into the page in FIG. 12B and downward dispensing in FIG. 12D) but in a more focused manner where the directionality of the arrows generally do not deviate from one another and are in the same direction. In FIGS. 12A through 12D, dispensing preventions are shown in a variety of other directions and locations except where dispensing is allowed.

Turning again to FIGS. 12E and 12F, a multidirectional dispensing operation is shown (e.g., generally into and out of the page in FIG. 12E and upward and downward in FIG. 12F). Distribution of dispensing is shown in a focused manner (similar to FIGS. 12C and 12D) in multiple directions. As is indicated by the number of arrows, dispensing out of the page and FIG. 12E and upward in FIG. 12F is less than dispensing into the page in FIG. 12E and downward in FIG. 12F. Although shown in the same focused manner of dispensing in multiple directions, it should be appreciated that some examples include dispensing in the sprayed manner in one direction and a focused manner in another direction. It should also be appreciated that dispensing may be the same amount in each direction of dispensing or may be different amounts in each direction of dispensing. As discussed above, an amount of dispensing can be proportional to permeability at the dispensing portion of the implantable medical system 9000. As previously described, FIGS. 12G and 12H show a similar dispensing operation (generally into and out of the page in FIG. 12G and upward and downward dispensing in FIG. 12H) but in a more sprayed manner (similar to FIGS. 12A and 12B) in multiple directions. In FIGS. 12E through 12G, dispensing preventions are shown in a variety of other directions and locations except where dispensing is allowed.

As noted above, FIGS. 12I-12P shows a Configuration B of the reservoir 9100 where the reservoir 9100 includes a main portion 9101 and a delivery arm 9103. Dispensing with respect to the main portion 9101 in these examples can be similar to those discussed above with reference to FIGS. 12A-12H. The main portion 9101 of the implantable medical system 9000 can be similar to those discussed with respect to FIGS. 12A-12H. The examples shown in FIGS. 12I-12P can, in addition or in alternative, include dispensing from the delivery arm 9103 as is further discussed below. While specific examples are discussed below, it should be appreciated that the skilled artisan would recognize many other examples and combinations thereof in light of this disclosure.

Turning to FIGS. 121 and 12J, a multidirectional dispensing operation at the delivery arm 9103 is shown. In this regard, dispensing at the delivery arm 9103 can occur in a variety of directions (e.g., at a tip portion of the delivery arm 9103 and along the length of the delivery arm 9103). Specific to this example, no dispensing occurs at the main portion 9101 of the implantable medical system 9000. In contrast, in the example shown in FIGS. 12M and 12N both the delivery arm 9103 and the main portion 9101 are configured to dispense in multiple directions. For instance, the main portion 9101 can dispense medicament in a similar manner as discussed with respect to FIGS. 12G and 12H, and the delivery arm 9103 can dispense medicament in a similar manner as discussed with respect to FIGS. 121 and 12J.

As previously discussed with reference to FIGS. 12K and 12L, a guided multidirectional dispensing operation at the delivery arm 9103 is shown. In this regard, dispensing at the delivery arm 9103 can occur in a variety of directions at only a tip portion of the delivery arm 9103. Specific to this example, no dispensing occurs at the main portion 9101 of the implantable medical system 9000 or along the length of the delivery arm 9103 separate from the tip portion. In contrast, in the example shown in FIGS. 12O and 12P the delivery arm 9103 is configured for guided multidirectional dispensing, and the main portion 9101 is configured for a focused dispensing. For instance, the main portion 9101 can dispense medicament in a similar manner as discussed with respect to FIGS. 12C and 12D, and the delivery arm 9103 can dispense medicament in a similar manner as discussed with respect to FIGS. 121 and 12J. Any of the above configurations of the implantable medical system 100 and dispensing mechanisms may be applied for use in treating presbyopia,

Under these circumstances, the medicament delivery system (e.g., in Configurations A and C) can be arranged at the anterior location of the eye 5000 and optionally (e.g., in Configurations B and D) in the anterior chamber 5006 of the eye 5000. Example API classes include miotics such as pilocarpine.

In some embodiments, the various medicament delivery systems discussed herein are shaped as thin, puck-shaped members. The medicament delivery systems may include thicknesses between exterior opposing surfaces of the (e.g., a distance measured between the second face 1104 of the first stratum 1100 and the second face 1204 of the second stratum 1200) of less than or equal to half of a millimeter (0.5 mm), such as between one-tenth of a millimeter (0.1 mm) and half of a millimeter (0.5 mm), for example although a variety of dimensions are contemplated. For example, given differing anatomies of the human body, the medicament delivery systems may exceed half of a millimeter (0.5 mm) without departing from the spirit or scope of the present disclosure provided that the thickness does not substantially interfere with normal eye functioning (e.g., pivoting and blinking).

In some embodiments, the various medicament delivery systems disclosed herein may have diameters (or widths across a major axis) in the range of five (5) millimeters to fifteen (15) millimeters. In a particular embodiment, the medicament delivery system disclosed herein may have a diameter (or width across a major axis) of ten (10) millimeters. In those embodiments where the medicament delivery systems are ovulary shaped, the medicament delivery systems may include a major dimension (e.g., of the oval) of up to about thirty (30) millimeters and corresponding minor dimension of up to about ten (10) millimeters. Though, as discussed above, given differing anatomies of the human body, the medicament delivery systems may exceed such dimensions (e.g., fifteen (15), and ten (10) and thirty (30) millimeters) without departing from the spirit or scope of the present disclosure provided that the size does not substantially interfere with normal eye functioning (e.g., pivoting and blinking). Likewise, the medicament delivery systems disclosed herein may include diameters (or widths across a major axis) of less than five (5) millimeters without departing from the spirit or scope of the present disclosure provided that the medicament delivery systems are operable to elute a sufficient degree of medicament for absorption by the surrounding tissue. The shapes and sizes discussed herein should not be viewed as limiting.

Additionally, while some medicament delivery systems discussed above are described as including a single medicament reservoir, it is to be appreciated that any of the above-discussed medicament delivery systems may include multiple reservoirs. These reservoirs may be fluidly coupled or isolated from one another. In some embodiments, each reservoir may be configured to house a same or different medicament. Accordingly, in some embodiments, the medicament delivery systems discussed herein may be configured to deliver multiple different medicaments, either from the same reservoir, or from a plurality of different reservoirs.

Additionally, in various embodiments, it is to be appreciated that the medicament may be loaded onto or otherwise incorporated into bioabsorbable particles which help aid in metering the medicament. In some embodiments, the particles may be of a size where they can be made into a dispersion and injected or otherwise delivered to within a medicament reservoir in situ (e.g., while the medicament delivery system is implanted within the patient's eye). That is, in some examples, the medicament may be included in a fluid suspension of particles. In various embodiments, a filled or partially filled medicament microporous reservoir may be accessed in situ (e.g., via syringe or other suitable means) and the contents of the reservoir (e.g., particles) removed and/or reloaded with fresh particles to maintain a constant delivery of medication over time. In various embodiments, placement of the fluid suspension of particles within the medicament reservoir may be accomplished via a syringe or other suitable means of delivery.

In some embodiments, the medicament delivery systems discussed herein are configured such that the medicament microporous reservoir retains the particles in the medicament reservoir and permits the dispersion carrier fluid (e.g., water) to exit the reservoir through one or more strata of the medicament delivery system. For instance, in some embodiments, the material defining the medicament reservoir (e.g., one or more of the first, second, third, or fourth microporous layers) may include a microstructure that is configured to prevent the particles of the fluid suspension from passing through the material. For example, the first microporous layer may be configured to resist tissue ingrowth, and may include interstices, perforations, pores, channels, or combinations thereof that prevent the particles of the fluid suspension from passing through the material. In some embodiments, the particles are configured to break down or degrade over time such that the medicament (alone or in a solution with the fluid) can percolate, diffuse, or otherwise pass through one or more of the strata of the microporous reservoir from an interior of the medicament reservoir to an exterior of the medicament delivery system for absorption by the body. In some embodiments, a concentration of the solution of dispersion carrier fluid and particles may change or be changed over time. For example, particles may be added to the dispersion carrier fluid (e.g., in situ) to increase a concentration of particles within the reservoir. Additionally or alternatively, dispersion carrier fluid may be added to reduce a concentration of particles within the reservoir.

In some embodiments, the various medicament delivery systems disclosed herein may additionally or alternatively include one or more structural spacers, such as one or more stents, struts, and/or reinforcing element. The one or more structural spacers may be incorporated into, integrated into, coupled to, or otherwise disposed within the reservoir to maintain a separation between those microporous layers forming the reservoir. Such structural spacers may be formed of any suitable biocompatible material (e.g., natural materials, or synthetic materials such as metals and polymers) discussed herein.

The inventive scope of this application has been described above both generically and with regard to specific embodiments and examples. It will be apparent to those skilled in the art that various modifications and variations can be made in the embodiments and examples without departing from the scope of the disclosure. Likewise, the various components discussed in the embodiments and examples discussed herein are combinable. Thus, it is intended that the embodiments and examples cover the modifications and variations of the inventive scope.

What is claimed is:

1. A method of treating diseases of the eye, the method comprising:
selecting an implantable delivery device that includes a first microporous material that is bonded to a second microporous material, the first microporous material having a first microporous layer including a plurality of pores sized to permit tissue ingrowth and a second microporous layer including a plurality of pores sized to permit tissue ingrowth, the second microporous material having a third microporous layer including a plurality of pores sized to resist tissue ingrowth and a fourth microporous layer including a plurality of pores sized to permit tissue ingrowth, the second microporous layer being bonded to the third microporous layer to thereby form a reservoir for receiving at least one medicament, wherein the first and second microporous materials are configured to meter a rate at which the medicament is dispensed from the reservoir when the delivery device is implanted;
filling the reservoir with a medicament for treating one or more diseases of the eye;
implanting the implantable delivery device at an implantation location; and
allowing the medicament to dispense from the reservoir to one or more treatment sites.

2. The method of claim 1, wherein the implantation location is an anterior reservoir location.

3. The method of claim 2, wherein the one or more eye diseases includes at least one of glaucoma, keratitis, dry eye, or presbyopia.

4. The method of claim 3, wherein when the one or more diseases is glaucoma, the medicament has an API class that includes at least one of prostaglandins, prostaglandin structural analogs, beta blockers, alpha agonist, or carbonic anhydrase inhibitors.

5. The method of claim 3, wherein when the one or more diseases is keratitis, the medicament has an API class that includes at least one of antibiotics, steroids, or antifungal agents.

6. The method of claim 3, wherein when the one or more diseases is dry eye, the medicament has an API class that corresponds to at least one of prostaglandins, beta blockers, alpha agonist, or carbonic anhydrase inhibitors.

7. The method of claim 3, wherein when the one or more diseases is presbyopia, the medicament has an API class that includes miotics.

8. The method of claim 1, wherein the implantation location is a posterior reservoir location.

9. The method of claim 8, wherein the one or more eye diseases includes at least one of macular degeneration, geographic atrophy lesion, macular edema, uveitis, retinitis, keratitis, retinoblastoma, central retinal vein occlusion, or branch retinal vein occlusion.

10. The method of claim 9, wherein when the one or more diseases is macular degeneration, the medicament has an API class that includes monoclonal antibodies, antibody mimetic proteins, peptides, or small binding molecules.

11. The method of claim 9, wherein when the one or more diseases is macular edema, the medicament has an API class that includes monoclonal antibodies, antibody mimetic proteins, peptides, small binding molecules, or steroids.

12. The method of claim 9, wherein when the one or more diseases is uveitis, the medicament has an API class that includes corticosteroid.

13. The method of claim 9, wherein when the one or more diseases is retinitis, the medicament has an API class that includes antibiotics or antivirals.

14. The method of claim 9, wherein when the one or more diseases is retinoblastoma, the medicament has an API class that includes cytotoxic chemotherapy compounds.

15. The method of claim 9, wherein when the one or more diseases is central retinal vein occlusion or branch retinal vein occlusion, the medicament has an API class that includes monoclonal antibodies or steroids.

16. The method of claim 1, wherein the implantation location is an anterior-posterior reservoir location.

17. The method of claim 16, wherein the one or more eye diseases includes at least one of macular degeneration, geographic atrophy lesion, macular edema, uveitis, retinitis, central retinal vein occlusion, or branch retinal vein occlusion.

18. The method of claim 17, wherein when the one or more diseases is macular degeneration, the medicament has an API class that includes monoclonal antibodies.

19. An implantable medical device pre-loaded with a therapeutic agent, the implantable medical device comprising:
a first microporous material bonded to a second microporous layer to define a reservoir disposed therebetween;
the reservoir configured for containing the therapeutic agent, and the implantable medical device configured to release the therapeutic agent at a predetermined rate; and
wherein the therapeutic agent is at least one of latanoprost, timolol, brimonidine, or dorzolamide.

20. An implantable medical device for delivering a therapeutic agent for treatment of a disease, the implantable medical device comprising:
a first microporous material bonded to a second microporous layer to define a reservoir disposed therebetween;
the reservoir configured for containing a pharmaceutical composition and to deliver the therapeutic agent to a target site for treating the disease; and
wherein the treatment is configured for treating one or more of glaucoma, macular degeneration, macular edema, retinitis, retinoblastoma, retinal vein occlusions, keratitis, and dry eye.
